(19) United States
(12) Patent Application Publication
(10) Pub. No.: US 2026/0218286 A1
(43) Pub. Date: Jul. 30, 2026

(54) METHODS AND COMPOSITIONS FOR AMPLIFYING METHYLATED TARGET DNA MOLECULES

(71) Applicant: Natera, Inc., San Carlos, CA (US)

(72) Inventors: Ting Wang, San Carlos, CA (US); Trupti KAWLI, San Carlos, CA (US); Ryan SWENERTON, Millbrae, CA (US); Bernhard ZIMMERMANN, Manteca, CA (US)

(73) Assignee: Natera, Inc., San Carlos, CA (US)

(21) Appl. No.: 19/146,167

(22) PCT Filed: Jan. 2, 2024
(86) PCT No.: PCT/US2024/010058

§ 371 Date: Jul. 7, 2025

Related U.S. Application Data
(60) Provisional application No. 63/437,016, filed on Jan. 4, 2023.

(51) Int. Cl. C12Q 1/686; C12Q 1/48; C12Q 1/6806; C12Q 1/6813; C12Q 1/6851; C12Q 1/6855; C12Q 1/6869; C12Q 1/6886; C40B 70/00

(52) U.S. Cl. CPC C12Q 1/686; C12Q 1/48; C12Q 1/6806; C12Q 1/6813; C12Q 1/6851; C12Q 1/6855; C12Q 1/6869; C12Q 1/6886; C12Y 207/07; C40B 70/00; C12Q 2600/154

(57) ABSTRACT

The present disclosure provides methods and compositions for preparing DNA molecules useful for determining a methylation status of selected regions of the DNA molecules. The methods utilize methylation sensitive restriction enzymes, and typically include one or more amplifications or a selective enrichment of target regions.

### CROSS-REFERENCE TO RELATED APPLICATIONS

This application claims the benefit of U.S. Provisional Application Ser. No. 63/437,016, filed Jan. 4, 2023, hereby incorporated by reference in its entirety.

### TECHNICAL FIELD

The present disclosure relates generally to methods for preparing and analyzing DNA molecules, and, more particularly, to methods for preparing and analyzing DNA molecules that include one or more methylation sites.

### BACKGROUND

Currently there is a need for improved methods for preparing and quantifying small amounts of DNA molecules in samples, such as circulating free DNA (cfDNA) carrying epigenetic alterations, such as changes in methylation status or patterns. There is a need for sensitive and efficient methods for preparing and quantifying small amounts of differentially methylated DNA molecules of interest in a sample.

There is a need for improved methods for preparing and quantifying small amounts of DNA molecules that have increased specificity and sensitivity for differentiating between DNA from different origins, such as tumor and non-tumor DNA. There is a need for improved methods for preparing and quantifying small amounts of DNA molecules, such as cfDNA, that allow the detection of tumor DNA based on epigenetic alterations with improved signal-to-noise resolution. There is a need for improved methods for preparing and quantifying small amounts of DNA molecules, such as cfDNA, to increase performance characteristics. There is a need for improved methods for preparing and quantifying small amounts of methylated DNA molecules that offer more flexibility for primer/probe design, not limited by the location of the restriction enzyme recognition sites. There is a need for improved methods for preparing and quantifying small amounts of methylated DNA molecules that can be used for many applications, such as downstream applications, in addition to detecting and/or quantifying methylated DNA molecules of interest.

While DNA methylation can be detected and analyzed using bisulfite conversion and sequencing, there is a need for improved methods for preparing and quantifying small amounts of methylated DNA molecules that not only are less destructive of sample DNA molecules, but that reduce the cost of the method, for example by only sequencing methylated fragments. There is a need for improved methods for preparing and quantifying small amounts of methylated DNA molecules that are faster, more cost effective, and more amendable to sequencing.

### SUMMARY

To overcome the above-mentioned and additional problems in the art, the present disclosure provides methods for determining a methylation status of selected regions of the DNA molecules. The methods provided herein allow detection of multiple co-methylated CpG sites and in illustrative embodiments, at multiple CpG islands, from small amounts of DNA molecules, such as circulating tumor DNA (ctDNA), to take advantage of the fact that in cancer cells, CpG sites in CpG islands at regulatory regions are predominantly co-methylated. The methods provided herein can also infer/use the methylation status of adjacent CpG sites not inside a particular target region, which increases detection specificity and allows more flexibility in primer/probe design. The methods provided herein allow selective pre-amplification of only fully methylated DNA fragments, thereby enriching DNA molecules potentially of interest. The methods provided herein increase performance characteristics in differentiating small amounts of cfDNA of different origins, such as differentiating ctDNA from cfDNA not of tumor origin.

Provided herein in one aspect is a method for preparing deoxyribonucleic acid (DNA) molecules useful for determining a methylation status of a genomic region of interest, said method comprising:
a) ligating adapters to sample DNA molecules obtained or derived from a first sample from a first subject, thereby forming a plurality of adapted DNA molecules comprising adapted DNA molecules having methylation sensitive restriction enzyme (MSRE) recognition sites;
b) contacting the plurality of adapted DNA molecules with one or more MSREs, thereby generating MSRE-exposed, adapted DNA molecules, wherein at least a first plurality of the MSRE-exposed, adapted DNA molecules having one or more unmethylated MSRE recognition sites are cleaved by at least one of the MSREs, and wherein a second plurality of MSRE-exposed adapted DNA molecules are uncleaved by any of the MSREs, thus forming MSRE-exposed, uncleaved adapted DNA molecules; and
c) performing one or more amplification reactions to amplify one or more target regions from the MSRE-exposed, uncleaved adapted DNA molecules or copies thereof, wherein target region amplicons are formed if the one or more target regions are present in the MSRE-exposed, uncleaved adapted DNA molecules.

A target region can be any genomic region of interest or any portions thereof. In some embodiments, the methods disclosed herein offer increased flexibility in selecting or defining target regions. In illustrative examples, target regions can be genomic regions where epigenetic changes, such as changes in DNA methylation, are associated with or indicative of formation or presence of cancer, and can, for example, include promoter regions of tumor suppressor genes, and in some embodiments DNA methylation markers for a specific cancer type. In some embodiments, each prospective target region amplicon has one or more MSRE recognition sites. In some embodiments, each prospective target region amplicon has two or more MSRE recognition sites. In some embodiments, at least 10% of the prospective target region amplicons each comprises at least two MSRE recognition sites. In some embodiments, at least 15%, 20%, 25%, 30%, or 35% of the prospective target region amplicons each comprises at least two MSRE recognition sites. In some embodiments, methods as described herein can include at least one prospective target region amplicon comprising at least 3, 4, 5, 6, 10, 15, 20, 25, 30, 35, or 40 MSRE recognition sites. In some embodiments, methods as described herein can include at least one prospective target region amplicon comprising between 2 and 45 MSRE recognition sites.

In some embodiments, the sample DNA molecules are circulating free DNA (cfDNA). In some embodiments, one or more target region amplicons are generated by one or more polymerase chain reactions (PCRs) using one or more primer pairs, and in illustrative embodiments at least one primer of each of the one or more primer pairs is a target-specific primer designed to bind to a specific nucleic acid sequence at or near, typically within, a target region. In some embodiments, one or more target region amplicons are generated using one or more capture probes designed to hybridize to one or more target regions. In some embodiments, one or more target region amplicons are generated by one or more linked target capture (LTC) reactions using one or more probe-dependent primer pairs. In some embodiments, the method further comprises detecting the one or more target region amplicons. In some embodiments, the one or more target regions are a set of target regions, and in certain embodiments the one or more primer pairs are a set of primer pairs, each being configured for amplifying one target region of the set. In some embodiments, the method further comprises quantifying an amount for at least one of the one or more target region amplicons.

Further details regarding aspects and embodiments of the present disclosure are provided throughout this patent application. Sections and section headers are for ease of reading and are not intended to limit combinations of disclosure, such as methods, compositions, and kits or functional elements therein across sections. Further details regarding aspects and embodiments of the present disclosure are provided throughout this patent application. Sections and section headers are for ease of reading and are not intended to limit combinations of disclosure, such as methods, compositions, or other functional elements therein across sections.

### BRIEF DESCRIPTION OF THE DRAWINGS

FIG. 1 illustrates an exemplary, non-limiting workflow for preparing DNA molecules having methylation-sensitive restriction enzyme (MSRE) recognition sites. Optional steps are indicated by dashed boxes. In FIG. 1, adapter molecules are ligated to DNA fragments obtained or derived from a sample (120). The adapted DNA fragments are then contacted with a mixture of MSREs (130). Targeted amplification (150) or selective enrichment (152) is then performed on the MSRE treated, adapted DNA molecules to form target region amplicons or an enriched subset of MSRE treated, adapted DNA molecules, respectively, that include a target region or a portion of the target region. Optional steps include extraction or isolation of DNA fragments prior to ligation (110). Optionally, after MSRE treatment, a universal amplification step (140) can be performed, and is typically performed, prior to a targeted PCR or selective enrichment step. Further, optionally, target region amplicons or enriched subsets can be detected and/or optionally quantified (160).

FIG. 2 illustrates a non-limiting exemplary workflow for a method of preparing circulating free DNA (cfDNA) for determining a methylation status of selected regions of the DNA molecules, for example from a subject having or suspected of having cancer. As shown in FIG. 2, the first step involves isolating cfDNA from a subject (111), followed by preparation of the cfDNA fragments, typically by modifying the cfDNA, for adapter ligation (115). In the next step, adapters are ligated to the modified cfDNA fragments (121), followed by contacting the adapted cfDNA fragments with a mixture of MSREs (130), to form MSRE treated, adapted cfDNA fragments. The MSRE treated, adapted cfDNA fragments are then amplified by performing a universal amplification step (140). Targeted amplification is then performed (150), and target region amplicons are then quantified using next-generation sequencing (NGS) (161).

FIG. 3 is a schematic illustration of the reagents and products of the workflow of FIG. 2. FIG. 3 (220) shows the adapted cfDNA fragments of step 121 of FIG. 2. As can be seen, the ligated adapters are Y adapters (224). The products of step 130 of the method of FIG. 2 are shown in FIG. 3 (230), where MSRE recognition sites containing methylated cytosine bases are not cleaved by the MSRE, while MSRE recognition sites having non-methylated cytosine bases are cleaved, leaving only uncleaved samples with intact universal primer sites on adapter portion of the adapted DNA molecules (232). In 250, targeted PCR primers (258, 262) are used to perform step 150 of FIG. 2, amplifying a target region (256, darkly shaded central region between and inclusive of the binding sites of the targeted PCR primer pair 258, 262) of universally amplified, MSRE exposed, adapted DNA molecules (252), in which the originally methylated cytosine bases (star-enclosed ‘C’) are no longer methylated post amplification. The resulting target region amplicons (254) include additional sequences added from non-target specific regions of the targeted PCR primers or additional primers, which are then quantified by sequencing or qPCR (260). Key: encircled mC: methylated cytosine base; encircled C: unmethylated cytosine base; star-enclosed C: originally methylated cytosine base.

FIG. 4 is a schematic illustration showing different non-limiting positions of PCR primer binding sites on an adapter-ligated DNA molecule including a target region (shown in hashed light grey), and methylated CpG sites both within and outside the target region.

FIG. 5A and FIG. 5B illustrate example workflows, Workflow 1 (W1) and Workflow 2 (W2), respectively, for determining the methylation status of sample cfDNA molecules from healthy donors and cancer patients. In FIG. 5A (Workflow 1), nucleic acid samples (411A) are first digested with a mixture of MSREs (430A), followed by a library preparation step, including blunt end repair, adapter ligation, and universal amplification (420A). In the following step, targeted PCR is performed on the library of universally amplified, adapter ligated, MSRE treated nucleic acid samples. FIG. 5B depicts Workflow 2 where nucleic acid samples (411B) are first blunt end repaired and adapter ligated (420B), followed by digestion with a mixture of MSREs (430B). The adapter ligated, MSRE treated sample DNA molecules are then universally amplified in step (440B), followed by targeted PCR (450B).

FIG. 6 is a schematic illustration of the reagents and products of Workflow 1. DNA fragments are treated with a mixture of MSREs, wherein DNA fragments with methylated MSRE recognition sites are uncleaved and DNA fragments with unmethylated MSRE recognition sites are cleaved (520A). The MSRE treated samples are then blunt end repaired and adapter ligated (530A). Targeted amplification (550A) is then performed on universally amplified MSRE treated, adapter ligated DNA molecules (552A) using targeted primer pairs (558A, 562A). Sample barcodes (553A) and NGS sequences (555A), which can be used for downstream NGS reactions, can also be added to the amplification products, resulting in amplified DNA molecules that include a target region flanked by NGS sequences and sample barcode (554A). Amplified DNA molecules are then quantified using qPCR and/or sequenced (560A).

FIG. 7 is a schematic illustration of the reagents and products of Workflow 2. In contrast to Workflow 1 in FIG. 6, FIG. 7 illustrates exemplary Workflow 2, where DNA fragments are blunt end repaired and adapter ligated (520B) prior to treatment with MSREs, wherein adapted DNA fragments with methylated MSRE recognition sites are uncleaved and adapted DNA fragments with unmethylated MSRE recognition sites are cleaved (530B). Following universal amplification, wherein only uncleaved adapted DNA fragments are amplified, the universally amplified MSRE treated, adapted DNA molecules (552B) are target amplified using targeted primer pairs (558B, 562B). Sample barcodes (553B) and NGS sequences (555B) can also be added to the amplification products. Resulting amplicons (554B) are then analyzed by sequencing or qPCR (560B).

FIG. 8 shows normalized depth of read (DOR) readout comparison for each MSRE in an MSRE cocktail with Workflow 1 (W1) or Workflow 2 (W2), using unmethylated lambda DNA samples treated with (MSRE) or without (Control) the MSRE cocktail. Data for targets containing one or more MSRE recognition sites were extracted and analyzed separately for each MSRE in the cocktail. All DOR were normalized to the median DOR of methylated pUC19 control.

FIG. 9 shows normalized DOR readout of targeted primer pool 1 of human targets for each MSRE recognition site from contrived 100% methylated (100% Methyl) or 0% methylated (Unmethyl) human gDNA samples treated with (MSRE) or without (Control) an MSRE cocktail using Workflow 1 (W1) or Workflow 2 (W2). All DOR were normalized to the median DOR of methylated pUC19 control.

FIG. 10 shows normalized DOR readout of targeted primer pool 2 of human targets for each MSRE recognition site from contrived 100% methylated (100% Methyl) or 0% methylated (Unmethyl) human gDNA samples treated with (MSRE) or without (Control) an MSRE cocktail using Workflow 1 (W1) or Workflow 2 (W2). All DOR were normalized to the median DOR of methylated pUC19 control.

FIG. 11 shows DOR heat map comparison of targeted primer pool 1 of human targets in cfDNA samples from colorectal cancer (CRC) patients vs. healthy donors using Workflow 1. Heat map of normalized DOR using targeted primer pool 1 and Workflow 1 protocol from samples of three individual CRC patients C1, C2 and C3 compared to 3 healthy individuals H1, H2, and H3 is shown. The variant allele frequencies (VAFs) for C1, C2, and C3 samples were at 25.31%, 20.52%, and 3.18% respectively. 100% methylated human gDNA and unmethylated human gDNA controls are shown in columns 1 and 2, respectively. All DOR were normalized to the median DOR of human non-MSRE control.

FIG. 12 shows DOR heat map comparison of Workflow 1 (W1) and Workflow 2 (W2) on cfDNA samples from CRC patients vs. healthy donors using two targeted primer pools. Samples were analyzed using either W1 or W2, and results from the two pools were combined. The VAFs for C1 and C2 were at 25.31% and 20.52%, respectively. All DOR were normalized to the median DOR of pUC19 control assays.

FIG. 13 shows DOR heat map comparison of Workflow 1 (W1) and Workflow 2 (W2) on cfDNA samples with lower VAFs (3.19% in W1, 2.51% or 1.78% in W2) from CRC patients vs. cfDNA samples from healthy donors using two targeted primer pools. All DOR were normalized to the median DOR of pUC19 control assays.

FIG. 14A and FIG. 14B show DOR heat map comparisons of targeted primer pool 1 (FIG. 14A) and pool 2 (FIG. 14B) with Workflow 2 on cfDNA samples from 13 CRC patients and cfDNA samples from 13 healthy donors. All DOR were normalized to the median DOR of human non-MSRE control.

FIG. 15 shows correlation of differential methylation level with SNV VAF. Higher DOR is associated with higher SNV VAF using top 40 targets.

FIG. 16A and FIG. 16B show normalized DOR in CRC positive, negative samples, and healthy samples. X-axis shows each target of pool 1 (A) and pool 2 (B) and Y-axis shows the normalized DOR for each group per target (Sample assay DOR/total reads). The target assays in the x-axis were sorted by ratio of CRC mean DOR vs healthy mean DOR. Solid lines is mean of normalized DOR, and shading is +/−standard error.

FIG. 17A and FIG. 17B show normalized DOR among top targets. Samples are grouped by SNV VAF range. (A) Grouping CRC samples with various SNV VAF range vs healthy samples using top 10 targets (ranked by Gini importance). (B) Grouping CRC samples with various SNV VAF range vs healthy samples using top 20 targets (ranked by Gini importance). Solid lines represent mean of normalized DOR, and shading is +/−standard error.

FIG. 18A and FIG. 18B show heat maps to compare normalized DOR between healthy/negative samples vs CRC samples for top targets of both pool 1 and pool 2. (A) Each column indicates per sample and each row represents each target among top 10 assays. (B) Each column indicates per sample and each row represents each target among top 20 assays. Values are normalized DOR.

FIG. 19A-FIG. 19D show ROC curve of the test set for different target groups. Random forest models were generated using (A) all targets; (B) top 20 targets in pool 1 and pool 2; C) top 20 targets in only pool 1; and (D) top 20 high targets in only pool 2. Target ranking was based on ratio of CRC mean DOR/healthy mean DOR.

FIG. 20 shows numbers of MSRE cut sites per assay associated with its CRC/healthy DOR ratio. The CRC/healthy ratio was calculated by mean DOR (CRC)/mean DOR (healthy) and the ratio rank is ordered by higher DOR ratio to low DOR ratio. Highest DOR ratio=Rank 1.

FIG. 21 illustrates an overview of the MSRE+Hybrid capture workflow. Nucleic acid samples (411C) are first blunt end repaired and adapter ligated (420C), followed by digestion with a mixture of MSREs (430C). The adapter ligated, MSRE treated sample DNA molecules are then universally amplified in step (440C), followed by barcoding PCR (460C). Libraries are normalized and pooled (470C) and then captured using the MSRE HC panel and amplified.

FIG. 22 shows normalized DOR in CRC positive, negative, and healthy samples. X-axis shows each target and Y-axis shows the normalized DOR for each group per target. The target assays in the x-axis were sorted by ratio of CRC mean DOR vs healthy mean DOR. Solid lines is mean of normalized DOR, and shading is +/−standard error. All DOR were normalized to the median DOR of human non-MSRE control.

FIG. 23A and FIG. 23B show normalized DOR for top targets. Samples are grouped by SNV VAF range. A) Grouping CRC samples with various SNV VAF range vs healthy samples using top 20 targets (ranked by CRC/healthy DOR ratio). B) Grouping CRC samples with various SNV VAF range vs healthy samples using top 40 targets (ranked by CRC/healthy DOR ratio). Solid lines are mean of normalized DOR, and shading is +/−standard error.

FIG. 24A and FIG. 24B show heat maps to compare normalized DOR between healthy/negative samples vs CRC samples. Each column indicates per sample and each row represents each target among top 20 (FIG. 24A) or top 40 (FIG. 24B) assays. Values are normalized DOR. Across targets, CRC samples can be differentiated from negative and healthy samples.

FIG. 25 shows the ROC curve of the test set. A) MSRE+Hybrid capture workflow. Random forest models were generated using top 40 high DOR ratio targets; B) MSRE+mPCR workflow. Random forest models were generated using top 20 high DOR ratio targets.

FIG. 26 shows numbers of MSRE cut sites per assay associated with its CRC/healthy DOR ratio. Not all assays with multiple MSRE cut sites provided high CRC/healthy DOR ratio for differentiation.

FIG. 27A and FIG. 27B show common good targets among HC and mPCR. A) Cross-checking top 40 targets using HC with top 40 targets from previous mPCR studies (Example 1 and 2) using the same set of targets, 24/40 HC targets are found in both mPCR studies and 36/40 are found in at least one mPCR studies. B) Among top 24 common good target assays, 23/24 targets contain more than 10 cut sites.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for DNA molecules or polypeptides are approximate, and are provided for description.

Further, ranges provided herein are understood to be shorthand for all of the values within the range. For example, a range of 1 to 50 is understood to include any number, combination of numbers, or sub-range from the group consisting, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 1 to 49, 1 to 25, 1.7 to 31.9, and so forth (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. When multiple low and multiple high values for ranges are given that overlap, a skilled artisan will recognize that a selected range will include a low value that is less than the high value.

As used herein, “about” or “consisting essentially of” mean±10% of the indicated range, value, or structure, unless otherwise indicated. As used herein, the terms “include” and “comprise” are open ended and are used synonymously. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

Although methods and materials similar or equivalent to those described herein can be used in the practice or testing of the present disclosure, suitable methods and materials are described below. All publications, patent applications, patents, and other references mentioned herein are incorporated by reference in their entireties. In case of conflict, the present specification, including explanations of terms, will control. In addition, the materials, methods, and examples are illustrative only and not intended to be limiting.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

### DETAILED DESCRIPTION

Both global and local epigenetic changes are widely regarded as a hallmark of cancer. Alterations include a global decrease in overall CpG methylation levels coupled with discrete regions of hypermethylation, typically in CpG islands located in the promoter regions of tumor suppressor genes. Hypermethylation has been associated with cancer progression and the silencing of growth regulating genes and tumor suppressor genes. As a result, a growing number of DNA methylation biomarkers are being utilized in the development of novel assays for monitoring cancer progression, treatment response and early detection. It is possible to detect the presence of cancer through the analysis of the methylation status of specific CpG sites that are predominantly methylated in DNA from tumor cells, including circulating tumor DNA (ctDNA) released from tumor cells.

The present disclosure addresses many long-felt needs and long-standing problems in the art, such as, but not limited to, those mentioned in the Background section herein. For example, methods are provided herein for preparing deoxyribonucleic acid (DNA) molecules useful for determining a methylation status of a genomic region of interest. Such methods can be used to detect methylated DNA molecules of interest, and demonstrate better specificity for detecting methylated DNA molecules of interest and improved signal-to-noise resolution for low VAF ctDNA samples.

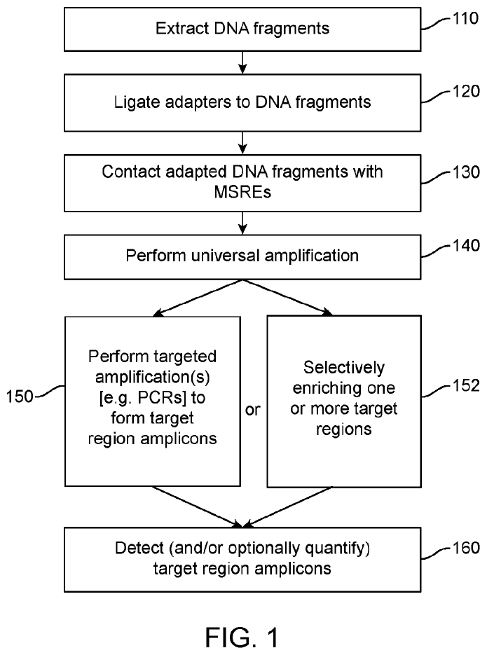

Accordingly, as illustrated in FIG. 1, provided herein in some aspects are methods for preparing deoxyribonucleic acid (DNA) molecules that have a methylation-sensitive restriction enzyme (MSRE) recognition site. Such methods are useful, for example, for determining a methylation status of a genomic region of interest, for detecting methylated DNA molecules, for quantitating methylated DNA molecules, and/or for detecting ctDNA. The steps of such aspects are shown in boxes in FIG. 1, with optional steps in such aspects shown in dashed boxes. Such methods, in non-limiting examples, can include the following steps:
a) ligating adapters to sample DNA molecules obtained or derived from a first sample from a first subject (120), thereby forming adapted DNA molecules comprising adapted DNA molecules having methylation sensitive restriction enzyme (MSRE) recognition sites;
b) contacting the plurality of adapted DNA molecules with one or more MSREs (130), thereby generating MSRE-exposed, adapted DNA molecules, wherein at least a first plurality of the MSRE-exposed, adapted DNA molecules having one or more unmethylated MSRE recognition sites are cleaved by at least one of the MSREs, and wherein a second plurality of MSRE-exposed adapted DNA molecules are uncleaved by any of the MSREs, thus forming MSRE-exposed, uncleaved adapted DNA molecules; and
c) performing one or more amplification reactions (140, 150) to amplify one or more target regions from the MSRE-exposed, uncleaved adapted DNA molecules or copies thereof, wherein target region amplicons are formed if the one or more target regions are present in the MSRE-exposed, uncleaved adapted DNA molecules.

Typically, target region amplicons are generated by one or more polymerase chain reactions (PCRs) (140, 150) on the MSRE-exposed, uncleaved adapted DNA molecules. More specifically, typically, one or more PCRs are performed using one or more primer pairs, and wherein at least one primer of each of the one or more primer pairs is a target-specific primer designed to bind to a specific nucleic acid sequence at or near, typically within, a genomic region of interest (150). Typically, one or more target region amplicons are detected. More specifically, one or more of the one or more target regions are a set of target regions, and wherein the one or more primer pairs are a set of primer pairs, each being configured for amplifying one target region of the set. Alternatively or in addition, one or more subsets of MSRE-exposed, uncleaved adapted cfDNA having one or more target regions can be enriched, typically following an universal amplification step (140), using one or more hybrid capture probes that bind to a nucleic acid sequence at or near, typically within, a target region (152). Typically, one or more target region amplicons can be enriched. More specifically, one or more of the one or more target regions are a set of target regions, and wherein the one or more hybrid capture probes are a set of hybrid capture probes, each being configured for enrich one target region of the set.

Another illustrative method herein for preparing DNA molecules useful for determining a methylation status of selected regions of the DNA molecules, includes the following steps:
a) ligating adapters to circulating free DNA (cfDNA) obtained or derived from a first liquid sample, thereby forming a plurality of adapted cfDNA comprising adapted cfDNA having MSRE recognition sites;
b) contacting adapted cfDNA with one or more MSREs, thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA;
c) performing an amplification step to amplify a set of target regions from the amplified MSRE-exposed, uncleaved, adapted cfDNA, each target region having one or more MSRE recognition sites, wherein a target region of the set is amplified and thereby generating target region amplicons if the target region is present in one or more of the amplified MSRE-exposed, uncleaved, adapted cfDNA; and
d) quantifying an amount for at least some of the target region amplicons by performing a next-generation sequencing (NGS) reaction on clonally amplified target region amplicons, or amplicons derived therefrom. Such quantitating, in illustrative embodiments, provides a quantity of methylated target regions. Some, most, a vast majority of, almost all, or all non-methylated target regions are not amplified in the targeted amplification reaction since they are digested with the MSRE before the targeted amplification.

Methods herein can optionally include extracting or isolating DNA molecules from a subject (110), for example from a liquid sample in a subject, which in illustrative embodiments comprises cfDNA. Alternatively, DNA molecules extracted or derived from a subject can be fragmented, for example by shearing, before they are processed in exemplary methods herein. In certain illustrative embodiments, cfDNA is extracted or derived from a subject having, suspected of having, or who had cancer. In some embodiments, methylation of genomic DNA is analyzed in a tumor of such subject, optionally using methods herein, and methylation of cfDNA is analyzed from that same subject, typically using methods herein, to identify ctDNA in the cfDNA sample.

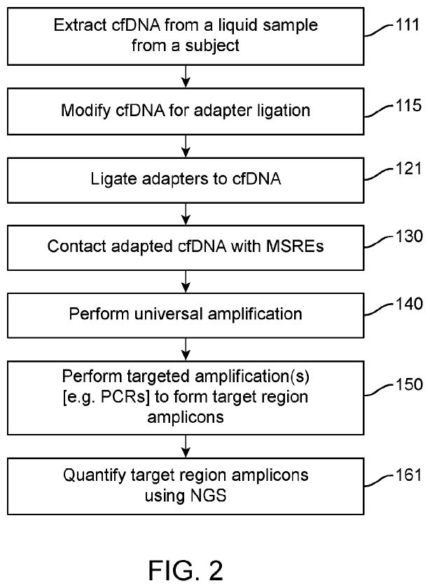

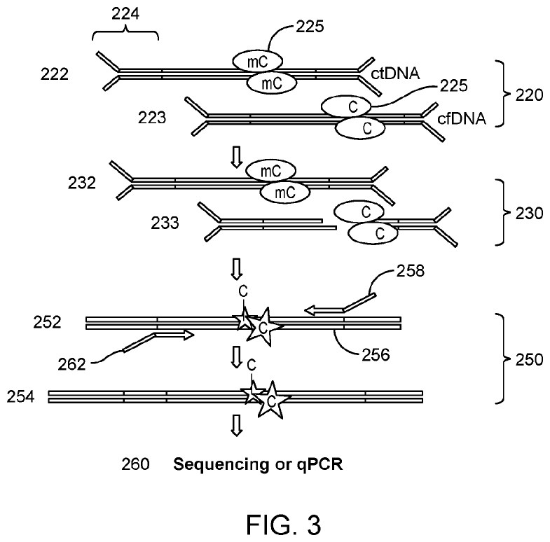

Accordingly, a non-limiting illustrative aspect of a method of preparing DNA molecules herein is shown in FIG. 2 and corresponding DNA reactants and products for such a method are shown in FIG. 3. Such non-limiting illustrative aspect includes the following steps:
a) extracting circulating free DNA (cfDNA) from a liquid (e.g. plasma) sample from a subject (111);
b) modifying cfDNA from the sample for adapter ligation (115) by preparing cfDNA derivatives; ligating adapters to cfDNA derivatives generated therefrom (121), thereby forming a plurality of adapted cfDNA comprising adapted cfDNA (220) having one, two, three, four or more MSRE recognition sites (one MSRE recognition site shown (225));
c) contacting the plurality of adapted cfDNA with one or more MSREs (130), thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA (232);
d) performing a first amplification step (140) with universal primers wherein at least a plurality of the MSRE exposed, uncleaved, adapted cfDNA are amplified (252);
e) performing a second amplification step to amplify a set of target regions (150) from the amplified MSRE-exposed, uncleaved, adapted cfDNA (252), each target region (darkly shaded central region (256) between and inclusive of the binding sites of the targeted PCR primer pair (258, 262)) having one or more MSRE recognition sites, wherein a target region of the set is amplified and thereby generating target region amplicons (254) if the target region is present in one or more of the amplified MSRE-exposed, uncleaved, adapted cfDNA; and
f) detecting and/or quantifying an amount for at least some of the target region amplicons of the set by performing a next-generation sequencing (NGS) reaction (161, 260) or a qPCR reaction (260) on clonally amplified target region amplicons, or amplicons derived therefrom.

In illustrative embodiments, such as but not limited to those shown in FIG. 2, methods herein can include modifying extracted or isolated DNA molecules (e.g. cfDNA) (115) to produce DNA molecules derived from sample DNA molecules (e.g. cfDNA) (e.g. modified DNA molecules such as modified cfDNA), before adapters are ligated to the modified DNA molecules (e.g. modified cfDNA). Such steps can include steps to prepare sample DNA molecules for adapter ligation, such as steps that can be performed during NGS library preparation. For example, such steps can include blunt-end repair and/or A-tailing, including addition of a poly- or single A tail (FIG. 2 (115)). The adapters used for ligation in illustrative embodiments are Y adapters (222), such as those used in NGS library preparation. Typically, the adapters do not include an MSRE recognition site.

In certain embodiments, adapted cfDNA (220) have two, three, four or more MSRE recognition sites. For example, in some embodiments, the adapted cfDNA may have 3, 4, 5, 6, 10, 15, 20, 25, 30, 35, 40 or more MSRE recognition sites. The adapted cfDNA (222, 223) of FIG. 3 each includes an MSRE recognition site (225). The MSRE recognition site of the upper adapted cfDNA (222), which is ctDNA in this illustration, includes a CpG site with a methylated cytosine residue (illustrated as encircled “mC”), which blocks MSRE cleavage. In contrast, the CpG site in the MSRE recognition site of the lower adapted cfDNA (223) is not methylated (illustrated as encircled “C”).

Thus, upon contacting the adapted cfDNA with one or more MSREs that recognize an MSRE recognition site containing a CpG site (mC or C in FIG. 3), the adapted cfDNA/ctDNA having only methylated MSRE recognition sites are uncleaved (232), whereas some, many, most, almost all, or all of the adapted cfDNA having one or more unmethylated MSRE recognition sites are cleaved (233) and thus such cleaved DNA molecules are not amplified during the subsequent amplification reactions.

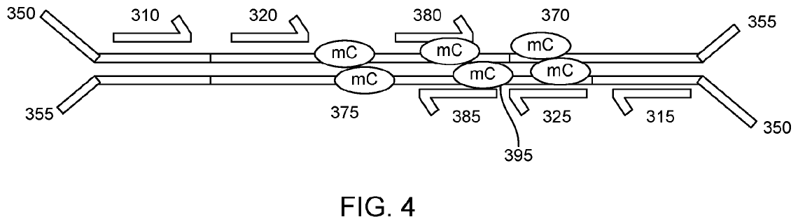

Methods herein typically include performing PCR using one or more targeted primer pairs that each includes at least one primer designed to bind to a specific sequence at or near, typically within, a target region of a sample nucleic acid and amplify one or more target regions in MSRE-exposed, adapted DNA molecules. The targeted PCR primer pairs typically define the ends of target region amplicons. As discussed in more detail herein, FIG. 4 provides various embodiments illustrating positions of primer binding sites on a DNA molecule that is subjected to exemplary methods herein. In some embodiments, the one or more target regions are a set of target regions, and the one or more primer pairs are a set of primer pairs, each being configured for amplifying one target region of the set.

In illustrative methods herein, at least one primer (e.g., 258) of each pair (e.g., 258 and 262) of primers used for a targeted amplification (e.g., PCR), or for an additional PCR(s) that is performed on amplicons generated by the targeted PCR, can optionally include a sample barcode/index. Furthermore, such primers for the targeted PCR or for the additional PCR(s), can be designed to contain sequences that can be utilized in subsequent sequencing step (i.e. “NGS sequences”). For example, such sequences can include NGS flow cell binding sites (e.g. Illumina P5 and P7 sequences) and/or NGS sequencing primer binding sites. Thus, primers used for the targeted PCR or for the additional PCR(s) may be designed to include NGS sequences, which can be used for downstream NGS analysis, and/or sample barcodes/indexes.

In certain illustrative embodiments, primers may be designed as probe-dependent primers, wherein targeted probes, designed to bind to specific nucleic acid sequences in or near regions of interest, are physically linked to universal primers designed to bind to universal primer binding sequences on the adapter portion of adapted DNA molecules (see e.g., Pel, et al. “Rapid and highly-specific generation of targeted DNA sequencing libraries enabled by linking capture probes with universal primers,” PLoS ONE 13(12):e0208283 (2018), incorporated by reference herein in its entirety). The primers of probe-dependent primers may also be designed to include NGS sequences, which can be used for downstream NGS analysis, and/or sample indexes.

In some embodiments, the method further comprises detecting and/or quantifying the target region amplicons, for example by sequencing or qPCR, thereby determining the methylation status of the one or more MSRE recognition sites within or near the one or more target regions on the sample DNA molecule (e.g. cfDNA). For example, an NGS reaction can be performed on clonally amplified target region amplicons generated by performing an additional amplification reaction that amplifies at least some of the target region amplicons, wherein in some embodiments the additional amplification reaction is a clonal amplification reaction, for example on an NGS substrate, to form clonally amplified target region amplicons. Furthermore, primers used to generate amplicons at any step, including for example primers (e.g. 262 and 258) used for a targeted amplification step, can include additional sequences, such as NGS sequences, or common sequences that are binding sites for one or more additional sets of primers used for one or more additional PCRs to amplify products of the targeted amplification to generate further amplicons. Such additional sets of primers that are capable of binding to the sequences added to targeted amplicon products can include additional sequences, such as NGS sequences, to generate amplicons that include additional sequences that surround the targeted region, as shown in the amplification products (254) of FIG. 3. Such targeted amplification(s) and/or additional amplification(s) can be performed in sequential cycling reactions, or within the same cycling reaction, and in illustrative embodiments are performed before an NGS clonal amplification reaction.

As provided in the illustrative example of FIG. 2, methods herein can optionally include one or more universal/library preamplification and/or amplification steps before and/or after targeted amplification steps (FIG. 2 (140)). Such methods include performing one or more PCR reactions using universal primers designed to bind to primer binding sites on an adapter portion of adapted DNA molecules and/or on universal primer binding sites on primers used for the targeted amplification(s) or an optional additional amplification(s).

In illustrative embodiments of methods herein, including but not limited to the method of FIG. 2, the liquid sample is a blood, plasma, serum, or urine sample, that typically contains cfDNA. In illustrative embodiments, the methylation status of the set of target regions is indicative of the presence or absence of ctDNA in the cfDNA sample, and typically indicative of the presence or absence of cancer in a subject.

### Sample Extraction and Enrichment of DNA Molecules

Samples that are useful for methods herein can be virtually any nucleic acid sample. In illustrative embodiments, the nucleic acid sample is extracted or isolated from a subject. Methods are known for extracting or isolating DNA molecules from a sample such as a tissue sample, and for illustrative embodiments herein, a liquid sample. Methods that are particularly useful in exemplary embodiments include methods for isolating circulating free DNA (cfDNA) from a liquid sample, and in illustrative embodiments from a blood, serum, urine, vitreous, sputum, saliva, tears, perspiration, feces, bile, lymph, cervical mucus, or semen sample, and in further illustrative embodiments, a plasma sample.

In certain illustrative embodiments, extracted or isolated DNA molecules can be enriched. Reagents, kits and associated methods for nucleic acid isolation and enrichment from biological samples, including for isolation of cfDNA from liquid samples, are generally available commercially. For example, isolation of cfDNA from a liquid (e.g. blood or blood derivative sample such as a serum or plasma sample) can involve binding DNA molecules from a sample to a matrix and isolating the DNA molecules in the presence of a solvent. In some embodiments, the method further comprises incubating the biological sample comprising DNA molecules with a protease, prior to contacting the DNA molecules to the matrix. In some embodiments, the method can further include the steps of washing the matrix with a wash buffer to remove impurities, and optionally, drying the matrix. Enriched nucleic acid samples can be eluted from the matrix with an elution buffer.

Other methods for nucleic acid isolation, for example cfDNA isolation, and optional enrichment of certain cfDNA can include ion exchange columns, or microfluidic devices, such as solid phase isolation, based on DNA capture by immobilized beads or functionalized surface. Additional methods include liquid phase isolation, utilizing an electric field, or chemical reagents, instead of a functionalized surface. Typically, in embodiments herein, isolation of cfDNA from a patient sample is performed using a DNA isolation kit (e.g. QIAamp Circulating Nucleic Acid kit (Qiagen)). In some embodiments, capture by hybridization with hybrid capture probes is used to preferentially enrich the DNA, for example using probes that bind to specific nucleic acid sequences at or near, typically within, target regions.

In some embodiments, cfDNA or their amplicons of certain sizes can be enriched before or after subjecting the cfDNA to methods herein. In some embodiments, size selection can be performed before the sequencing library preparation. In some embodiments, size selection can be performed after the sequencing library preparation and before sequencing. In some embodiments, size selection is performed on a sequencing-ready pool. Enriched cfDNA molecules can be, for example 50 to 1200 base pairs in length, 70 to 500 base pairs in length, 100 to 200 base pairs in length, or 130 to 170 base pairs in length. In some embodiments, the enriched cfDNA molecules are from 50 to 200 bp in length. In some embodiments, the enriched cfDNA molecules are between 60 and 200 bp in length, between 60 and 150 bp in length, or between 60 and 100 bp in length before the enriched cfDNA molecules, or derivatives thereof, are ligated to adapters in methods herein. In some embodiments, the enriched cfDNA molecules are less than 150, 100, 90, 75, or 50 bp in length before they are ligated to adapters. Such enrichment methods can be performed for example using the methods of WO2018156418 A1, Stray, et al. (incorporated herein by reference in its entirety).

In some embodiments, the sample is enriched for tumor DNA molecules, which are typically less than 160 bp and have a peak at about 145 bp in length. In illustrative embodiments, the enriched nucleic acid sample is circulating tumor DNA (ctDNA) or amplicons thereof. In such embodiments, the enriched nucleic acid is less than 160, 150, 145, 120, 100, 90, 75, or 50 bp in length. In some embodiments, size selection is used to filter out cfDNA molecules carrying mutations derived from clonal hematopoiesis of indeterminate potential (CHIP) but not tumor-derived mutations, which are typically longer than ctDNA molecules, at about 165 bp.

DNA methylation biomarkers are increasingly being utilized in the development of novel assays for use in women's health and/or organ health. For example, methylation profiling is being used in non-invasive prenatal testing (NIPT) for monitoring of placental and fetal epigenomic changes, pre-symptomatic detection of preterm birth, preeclampsia, placental insufficiency, and fetal growth restriction. Methylation markers can also be indicative of congenital diseases of the fetus. Furthermore, methylation biomarkers are also used for monitoring of organ health such as predicting and monitoring organ rejection in transplant patients, monitoring of immune changes in transplant rejection, and for monitoring of organ health in high risk or predisposed individuals. Accordingly, the sample described herein may be a maternal sample, a fetal sample, or a sample from a transplant patient.

In some embodiments, the sample is enriched for fetal DNA molecules. In illustrative embodiments of such embodiments, the enriched nucleic acid sample is fetal circulating free DNA or amplicons thereof. In such embodiments, the enriched nucleic acid is between 100 bp and 220 bp in length. In some embodiments, the length of the nucleic acid sample of fetal cfDNA ranges from 100 bp to 200 bp, from 120 bp to 180 bp, from 140 bp to 160 bp, from 150 bp to 170 bp, from 160 to 190 bp, or from 170 bp to 220 bp.

In some embodiments, the sample is enriched for transplant donor DNA molecules. In illustrative embodiments of such embodiments, the enriched nucleic acid sample is transplant donor circulating free DNA or amplicons thereof. In such embodiments, the enriched nucleic acid is between 100 bp and 220 bp in length. In some embodiments, the length of the nucleic acid sample of transplant donor cfDNA ranges from 100 bp to 200 bp, from 120 bp to 180 bp, from 140 bp to 160 bp, from 150 bp to 170 bp, from 160 bp to 190 bp, or from 170 bp to 220 bp.

### Subjects

Subjects used in methods herein can be virtually any animal, in illustrative embodiments a mammal, and in further illustrative embodiments, a human. In some embodiments, the subject is suspected or at risk of having a disease, in some embodiments, cancer. In some embodiments, the subject is a pregnant female. In some embodiments, the subject is a subject comprising an organ from another individual.

In embodiments where the subject has cancer, the cancer can be any type of cancer provided that the genome of cancerous cells of the subject have portions of their genome that are differentially methylated compared to non-cancerous cells of the subject. Typically, some, most, almost all or all cancer cells of the subject have region(s) of their genome that are methylated that are not methylated in non-cancerous cells of the subject, or that are more methylated than non-cancerous cells. Thus, in some embodiments, the subject has one or more cancers (e.g. one cancer) including ovarian cancer, soft tissue sarcoma, peripheral T cell cancer, colorectal cancer, intrahepatic cholangiocarcinoma, glioblastoma, esophageal cancer, cutaneous T cell lymphoma, non-Hodgkin lymphoma, urothelial cancer, basal cell carcinoma, epithelioid sarcoma, pancreatic cancer, non-small cell lung carcinoma, Hodgkin lymphoma, renal cell carcinoma, mesothelioma, metastatic uveal melanoma, kidney cancer, blood cancer, HER2-expressing cancers, non-melanoma skin cancer, liposarcoma, hepatocellular carcinoma, small lymphocytic lymphoma, prostate cancer, breast cancer, anal cancer, marginal zone lymphoma, cutaneous squamous cell carcinoma, thyroid cancer, medullary thyroid cancer, triple-negative breast cancer, neuroendocrine prostate cancer, bladder cancer, paraganglioma, medulloblastoma, superficial basal cell carcinoma, head and neck squamous cell carcinoma, hematologic malignancies, melanoma, B-cell lymphoma, relapsed/refractory acute myeloid leukemia, angiosarcoma, bone sarcoma, refractory cervical cancer, cholangiocarcinoma, osteosarcoma, biliary tract cancer, castration-resistant prostate cancer, gastroesophageal adenocarcinomas, rhabdomyosarcoma, carcinoma, non-muscle invasive bladder cancer, uveal melanoma, small cell lung cancer, cervical cancer, primary open angle glaucoma, follicular lymphoma, synovial sarcoma, liver cancer, carcinosarcoma, leptomeningeal brain tumors, T-cell lymphoma, lymphoma, small cell lung cancer, mantle cell lymphoma, B-cell malignancies, endometrial cancer, myxoid/round cell liposarcoma, metastatic Merkel cell carcinoma, neuroblastoma, chronic lymphocytic leukemia, tenosynovial giant cell tumors, sarcoma, acute myeloid leukemia, skin cancer, nasopharyngeal carcinoma, relapsed/refractory Ewing sarcoma, bone cancer, glioma, salivary gland carcinoma, gastric cancer, benign tumor, low-grade serous ovarian cancer, metastatic breast cancer, multiple myeloma, diffuse large B cell lymphoma, relapsed/refractory lymphoma, metastatic colorectal cancer, advanced malignancies, and acute lymphoblastic leukemia.

In certain embodiments of methods herein, the subject has a cancer of the abdomen or abdominal wall, adrenal gland, anus, appendix, bladder, bone, brain, breast, cervix, chest wall, colon, diaphragm, duodenum, ear, endometrium, esophagus, fallopian tube, gallbladder, gastroesophageal junction, head and neck, kidney, larynx, liver, lung, lymph node, malignant effusions, mediastinum, nasal cavity, omentum, ovaries, pancreas, pancreatobiliary, parotid gland, pelvis, penis, pericardium, peritoneum, pleura, prostate, rectum, salivary gland, skin, small intestine, soft tissue, spleen, stomach, thyroid, tongue, trachea, ureter, uterus, vagina, vulva, and whipple resection. In illustrative embodiments, the cancer is selected from nasopharyngeal carcinoma, hepatocellular carcinoma, breast cancer, ovarian cancer, pancreatic cancer, colorectal cancer, lung cancer, oesophageal cancer, prostate cancer, bladder cancer, melanoma, and acute leukemia. In illustrative embodiments, the cancer is selected from colorectal cancer.

### Detection & Analysis

Methods as described herein typically include detecting and optionally quantifying nucleic acids, including DNA, cfDNA, amplified MSRE-exposed, uncleaved, adapted cfDNA, enriched subsets of DNA having target regions, and in illustrative embodiments, target region amplicons or amplicons derived therefrom. In some embodiments, cfDNA from a blood sample from the individual is analyzed. Not to be limited by theory, cfDNA is believed to be released from certain cells, such as cancer cells, for example when they undergo necrosis or apoptosis. In some embodiments, methods herein can be used to detect methylation in target regions or nucleic acid sequence of interest that is present in a small percentage of DNA in a sample, such as cfDNA, for example from a fetus, a cell from a donated organ, or in illustrative embodiments, a cancer cell.

In methods herein, at least some of the subsets of amplified MSRE-exposed, uncleaved, adapted cfDNA can be enriched using a set of hybrid capture probes to form at least some of the enriched subsets of amplified MSRE-exposed, uncleaved, adapted cfDNA before detecting or quantifying an amount for at least some of the enriched subsets. In methods that include detecting or quantifying the target region amplicons, such target region amplicons can be enriched using a set of hybrid capture probes before detecting or quantifying. In illustrative embodiments, methods here in can include detecting or quantifying the target region amplicons without a selective enrichment step.

In some embodiments, the method further comprises an additional amplification reaction that amplifies at least some of the amplified target region amplicons, or amplifies at least some of the enriched subsets of amplified MSRE-exposed, uncleaved adapted cfDNA, for detecting or quantifying. The additional amplification reaction can be, for example, a quantitative PCR (qPCR reaction) such as TAQMAN assay (LIFE TECHNOLOGIES), or an INVADER assay (THIRD WAVE TECHNOLOGIES), a digital PCR, or any other method for detecting and/or quantifying a target DNA, which typically herein comprise a target region that includes one or more MSRE sites.

In some embodiments, the additional amplification reaction is a clonal amplification reaction to form clonally amplified target region amplicons, or to form clonally amplified enriched subsets of amplified MSRE-exposed, uncleaved, adapted cfDNA. For detecting or quantifying at least some of the amplified and/or selectively enriched target region amplicons, methods herein can comprise i) performing an additional amplification reaction that amplifies at least some of the amplified target region amplicons, wherein the additional amplification reaction is a clonal amplification reaction to form clonally amplified target region amplicons, and ii) performing a next-generation sequencing reaction on the clonally amplified target region amplicons. For detecting or quantifying at least some of the enriched subsets of amplified MSRE-exposed, uncleaved adapted cfDNA, methods herein can comprise i) performing an additional amplification reaction that amplifies at least some of the enriched subsets of amplified MSRE-exposed, uncleaved adapted cfDNA, wherein the additional amplification reaction is a clonal amplification reaction to form clonally amplified enriched subsets of amplified MSRE-exposed, uncleaved, adapted cfDNA, and ii) performing a next-generation sequencing reaction on the clonally amplified enriched subsets of amplified MSRE-exposed, uncleaved, adapted cfDNA. The detecting or quantifying included in methods herein, can comprise performing a sequencing reaction on the clonally amplified target region amplicons.

In some embodiments, the sequencing reaction is a next-generation sequencing reaction. In methods herein, detecting or quantifying comprises counting sequence reads generated from clonally amplified target region amplicons. Quantifying can also comprise determining a depth of read per target region for at least some of the target regions. Depth of read for each of the target region can be normalized relative to a depth of read for a normalization sequence. DNA sequences used for normalization can be derived from genomic DNA or from control plasmids, and can contain no MSRE cut sites, or fully methylated MSRE cut sites, and will depend on the experimental conditions, such as selection of MSREs. DNA sequences used for normalization for quantitative methods herein, such as NGS, can be 100% methylated at MSRE cut sites or have no MSRE cut sites. The normalization sequence can be a 100% methylated contrived DNA molecule derived from a control genomic DNA sample or a pUC19 control plasmid. The normalization sequence can be derived from a lambda control plasmid having 100% methylated DNA. A fully methylated (100% methylated) synthetic sequence, for example, can also be considered as a normalization sequence. In some embodiments, the normalization sequence can be a non-MSRE DNA molecule having no MSRE recognition site for any of the MSRE or MSREs used. In some embodiments, non-MSRE sequences for normalization have no MSRE recognition site on the prospective amplicons. In some embodiments, non-MSRE sequences for normalization have no MSRE recognition site on and +/−200 bp, +/−150 bp, +/−100 bp, +/−50 bp, +/−40 bp, or +/−40 bp of the prospective amplicons. Such a non-MSRE DNA molecule can be derived from a first subject.

A non-MSRE DNA molecule as per methods herein can be a synthetic sequence. The normalization sequence can be a spike-in control DNA sample that is not subject to an MSRE digestion step. A spike-in control DNA sample can be a fully methylated genomic, plasmid, or synthetic DNA sample. A spike-in control sample used for normalization, in some embodiments, can be 0.00005%, 0.0001%, 0.005%, 0.001%, 0.05%, 0.1%, 0.2%, 0.5%, 0.7%, 0.8%, 1.0%, 1.2%, 1.4%, 1.6%, 1.8, 2%, or more fully methylated DNA by mass. A spike-in control sample can be fully methylated or non-MSRE DNA that range between 0.00005 to 2%, 0.0001 to 2%, 0.005 to 2%, 0.001 to 2%, 0.05 to 2%, 0.1 to 2%, 0.5 to 2%, 1 to 2%, 0.00005 to 1.5%, 0.00005 to 1.2%, 0.00005 to 1%, 0.00005 to 0.8%, 0.00005 to 0.5%, 0.00005 to 0.3%, or 0.00005 to 0.2% by weight. Methods herein, can include more than one, for example 2, 3, 4, 5, or more control or spike-in control sample. DNA sequencing techniques, particularly high throughput next-generation sequencing techniques (often referred to as massively parallel sequencing techniques) such as those employed in MYSEQ (ILLUMINA), HISEQ (ILLUMINA), ION TORRENT (LIFE TECHNOLOGIES), GENOME ANALYZER ILX (ILLUMINA), GS FLEX+(ROCHE 454) etc., can be used for quantitative measurements of the number of copies of a target region present after MSRE digestion, for example, but not limiting to, clonally amplified target region amplicons or clonally amplified enriched subsets of amplified MSRE-exposed, uncleaved, adapted cfDNA, and thus provide quantitative information regarding the number and/or amount of methylation in sample DNA molecules, for example, cfDNA. High throughput genetic sequencers are amenable to the use of barcoding (i.e., sample tagging with distinctive nucleic acid sequences) so as to identify specific samples from individuals thereby permitting the simultaneous analysis of multiple samples in a single run of the DNA sequencer. The number of times a given region of the genome in a library preparation (or other nucleic preparation of interest) is sequenced (number of reads) will be proportional to the number of copies of that sequence that was not digested by MSREs (i.e. was methylated). Methods as described herein that utilize NGS detection, in some embodiments can have an average depth of read of at least 200, 500, 1000, 2000, 2900, 3000, 3500, 4000, 5000, 10,000, 50,000, 75,000, 100,000, 130,000, 150,000, 175,000, or 200,000.

Methods herein can include analyzing data obtained from next-generation sequencing techniques. In some embodiments of methods herein, clonally amplified target region amplicons or clonally amplified enriched subsets of amplified MSRE-exposed, uncleaved, adapted cfDNA can be subjected to sequencing using next-generation sequencing techniques. Nucleic acid sequencing data can be generated for amplicons created by PCR, for example a multiplex targeted PCR (mPCR). In some embodiments, the multiplex PCR can be a tiled multiplex PCR. For a skilled artisan, algorithm design tools are available that can be used and/or adapted to analyze the sequencing data. In addition, those skilled in the art can determine appropriate parameters for measuring alignment to a consensus sequence and/or to a known target region sequence, including any algorithms needed to achieve maximal alignment over the length of the sequences being compared.

Sequencing reads can be demultiplexed using an in-house tool and mapped using the Burrows-Wheeler alignment software, Bwa mem function (BWA, Burrows-Wheeler Alignment Software (see Li H. and Durbin R. (2010) Fast and accurate long-read alignment with Burrows-Wheeler Transform. Bioinformatics.) on single end mode using pear merged reads to the hg19 genome. Amplification statistics QC can be performed by analyzing one or more of, but not limiting to, total reads, number of mapped reads, number of mapped reads on target, and number of reads counted.

Methods herein can include a background error model that can be constructed using normal, or healthy liquid samples, in illustrative embodiments, normal, or healthy plasma samples, which are sequenced on the same sequencing run to account for run-specific artifacts. In some embodiments, 5, 10, 15, 20, 25, 30, 40, 50, 100, 150, 200, 250, or more than 250 normal, or healthy liquid samples, in illustrative embodiments, plasma samples can be analyzed on the same sequencing run. The number of samples that can be sequenced on the same sequencing run can be in the range of 5 to 500, 5 to 400, 5 to 300, 5 to 250, 20 to 250, 30 to 250, 50 to 250, 75 to 250, 100 to 250, 50 to 500, or 100 to 500. Sample barcodes are used in illustrative embodiments. In some illustrative embodiments, 20, 25, 40, or 50 normal samples (e.g. plasma samples) can be analyzed on the same sequencing run. Outlier samples can be iteratively removed from the model to account for noise and contamination. In some embodiments, samples with a Z score of greater than 5, 6, 7, 8, 9, or 10 are removed from the data analysis. For each base substitution of every genomic loci, the DOR weighted mean and standard deviation of the error can be calculated.

Methods herein can include calculating percent identity that can be calculated by determining the number of matched positions in aligned DNA sequences, dividing the number of matched positions by the total number of aligned DNA sequences, and multiplying by 100. A matched position refers to a position in which identical nucleotides occur at the same position in aligned DNA sequences. The percent identity over a particular length can be determined by counting the number of matched positions over that length and dividing that number by the length followed by multiplying the resulting value by 100. A non-limiting example for calculating the percent identity, can be, if (i) a 500-nucleotide DNA target sequence is compared to a subject DNA sequence, (ii) an alignment program presents 200 nucleotides from the target DNA sequence aligned with a region of the subject DNA sequence where the first and last nucleotides of that 200-nucleotide region are matches, and (iii) the number of matches over those 200 aligned nucleotides is 180, then the 500-nucleotide nucleic acid target sequence contains a length of 200 and a sequence identity over that length of 90 percent (i.e., 180, 200×100=90).

In some embodiments, the uniformity in DOR can be measured using standard methods such as, but not limiting to, DOR slope, normalized median depth of read (nmDOR), or breadth of read (BOR). DOR slope represents the slope of the line in the linear portion of a list of loci sorted in descending DOR order. Closer to zero is better, as it represents a flat line. In some embodiments, the uniformity in DOR can be measured using the percent of reads in the 90^{th}-95^{th} percentile. For this measurement, the loci are sorted in descending DOR order. In illustrative embodiments, a DOR distribution using the 90^{th}-95^{th} percentile contains 5 percent of reads. The reads of all loci between the 90^{th} percentile and 95^{th} percentile can be counted and divided by the total reads for all loci.

In some embodiments, the magnitude of the DOR slope can be less than 0.005, 0.001, 0.0005, 0.0001, 0.00005, 0.00001, 0.000005, or 0.000001. The magnitude of the DOR slope can be between 0 and 0.005, such as 0.000001 to 0.005, such as between 0.000005 to 0.00001, 0.00001 to 0.00005, 0.00005 to 0.0001, 0.0001 to 0.0005, 0.0005 to 0.001, or 0.001 to 0.005. The percent of reads in the 90^{th}-95^{th} percentile can be between 0.2 and 9 percent, such as between 0.2 to 8 percent, 0.2 to 7 percent, 0.2 to 6 percent, 0.4 to 9 percent, 0.4 to 8 percent, 0.4 to 7 percent, 0.4 to 6 percent, 1 to 9 percent, 1 to 8 percent, 1 to 7 percent, 1 to 6 percent, 2 to 9 percent, 2 to 8 percent, 2 to 7 percent, 2 to 6 percent, 3 to 9 percent, 3 to 8 percent, 3 to 7 percent, 3 to 6 percent, 0.2 to 1.0 percent, 1 to 2 percent, 2 to 3 percent, 2 to 4 percent, 3 to 4 percent, 4 to 5 percent, 5 to 6 percent, or 6 to 8 percent, or 7 to 9 percent. In some embodiments of methods herein, the method or the amplification steps in the method can produce a composition comprising at least 100 different amplicons (e.g., at least 300, 500, 750, 1,000, 2,000, 5,000, 7,500, 10,000, 15,000, 19,000, 20,000, 25,000, 27,000, 28,000, 30,000, 40,000, 50,000, 75,000, or 100,000 non-identical amplicons) with the magnitude of the DOR slope in any of the ranges herein, or with a percent of reads in the 90^{th}-95^{th} percentile in any of the ranges herein. In some embodiments, different amplicons can range in between 100 to 500,000, 100 to 400,000, 100 to 300,000, 100 to 200,000, 100 to 100,000, 100 to 75,000, 100 to 50,000, 100 to 40,000, 100 to 30,000, 100 to 25,000, 100 to 20,000, or 100 to 15,000 non-identical amplicons.

In some embodiments of methods herein, in addition, or, in some embodiments, as an alternative to analyzing an altered (increased or decreased) methylation levels in a sample, one or more other factors can be analyzed if desired. These factors can be used to increase the accuracy of the diagnosis (such as determining the presence or absence of cancer or an increased risk for cancer, classifying the cancer, or staging the cancer) or prognosis. These factors can also be used to select a particular therapy or treatment regimen that is likely to be effective in the subject.

### Limits of Detection

Exemplary methods herein are to detect target region amplicons generated by a targeted amplification and/or selectively enriched, and used to determine the methylation status of one or more, in illustrative embodiments a plurality of, methylation sites of interest on a DNA molecule in a DNA sample. A target region can include 1, 2, 3, 4, 5, 6 or more CpG sites that are predominantly methylated or co-methylated, typically at a cytosine nucleotide, in cells of a certain origin (e.g. a cancer). For example, the target region can be all or a portion of a CpG island predominantly methylated in a tumor. In illustrative embodiments, sample DNA molecules comprising the target region are fragments of genomic DNA or circulating free DNA (cfDNA) of a subject. Target regions can be selectively amplified and/or selectively enriched using methods herein. Exemplary methods herein, in some embodiments, have a limit of detection of as low as 1.0%, 0.5%, 0.1%, 0.05%, 0.01%, 0.005%, or 0.001%, wherein the method is capable of detecting fully methylated DNA molecules present at 1.0%, 0.5%, 0.1%, 0.05%, 0.01%, 0.005%, or 0.001% (by mass) or more in a mixture of DNA molecules. In some embodiments, methods herein are capable of differentiating samples having 1.0%, 0.5%, 0.1%, 0.05%, 0.01%, 0.005%, or 0.001% (by mass) or more contrived fully methylated DNA molecules from samples having 0% of contrived fully methylated DNA molecules. Exemplary methods herein, in some embodiments, have a limit of detection of as low as 1.0%, 0.5%, 0.1%, 0.05%, 0.02%, 0.01%, 0.005%, 0.002%, or 0.001% differential methylation allele fraction (DMAF), which estimates the fraction of differential methylation alleles for circulating cell-free DNA across target regions.

In some embodiments, measurements can be adjusted for bias, such as bias due to differences in amplification efficiency or adjusted for sequencing errors. In some embodiments, differentiation between methylated and unmethylated samples can be analyzed using a control sample that is fully methylated, for example a fully methylated plasmid control (e.g. pUC19) for normalization of quantitative results. In some embodiments, the differentiation between the methylated samples recited in this paragraph can be achieved after normalization of a detected and typically quantified signal using one or more (e.g. 2, 3, 4, 5, or 6) controls that do not have an MSRE recognition site.

In certain embodiments, ctDNA is detected when target region amplicons are detected, since such target region amplicons are typically generated when MSRE recognition sites on sample DNA molecules are methylated, which is known to occur in the promoter region of tumor suppressor genes at early stages of carcinogenesis. In certain embodiments, the method is capable of detecting ctDNA when it is present in 50%, 45%, 40% 35%, 30%, 25%, 20%, 15%, 10%, 5%, 2.5%, 2%, 1%, 0.5%, 0.1%, 0.05%, 0.01%, 0.005%, 0.002%, 0.001%, or less of the circulating free DNA in a sample. In some embodiments, methods herein detect or are capable of detecting ctDNA from a sample when it is present at a range between 0.1%, 0.05%, 0.02%, 0.01%, 0.005%, 0.002%, or 0.001% on the low end and 50%, 45%, 40%, 35%, 30%, 25%, 20%, 15%, 10%, 5%, 2.5%, 1%, or 0.5% on the high end of the range of total cfDNA in the sample. Methods herein, in some embodiments, detect or are capable of detecting 5%, 4%, 3%, 2%, 1%, 0.5%, 0.1%, 0.05%, 0.01%, 0.005%, 0.002%, 0.001% or less circulating tumor DNA, as a percent of total circulating free DNA (cfDNA) from a sample. The percentage of ctDNA in a cfDNA sample can be determined or approximated by variant allele frequency (VAF) of one or more DNA variants, such as single nucleotide variants (“SNVs”), present in tumor cells but not in normal cells. (See e.g., WO2019200228, incorporated by reference herein in its entirety). VAF can be used as a surrogate measure of the percentage of ctDNA in the cfDNA sample. At least in colorectal cancer (CRC) patients, there is a strong correlation (about 0.9) between DMAF and VAF regardless of clinicopathologic features such as disease stage, histology, age, or sex. Methylation-based assays are a promising tool for cancer detection and quantifying the disease burden.

### Nucleic Acid Processing Before MSRE Treatment

Typically, methods herein include a step of ligating nucleic acid adapters to sample DNA molecules, or in illustrative embodiments to nucleic acid derivatives generated therefrom. The sample DNA molecules in illustrative embodiments are from a subject. In some embodiments, ligating nucleic acid adapters is preformed after sample DNA molecules are fragmented to form fragmented DNA molecules. Typically, methods include exposing sample DNA molecules to one or more polymerases or kinases, such as Klenow Large Fragment Polymerase and T4 polynucleotide kinas (PNK), as well as a ligase, such as T4 ligase. In some embodiments, sample DNA molecules or the fragmented DNA molecules, are exposed to one or more polymerases and/or kinases to generate the nucleic acid derivatives generated therefrom. In some embodiments, the method further comprises ligating adapters to the nucleic acid derivatives generated therefrom.

Typically, adapters are ligated to sample DNA molecules in illustrative methods herein. In illustrative embodiments, before such ligation, extracted or isolated DNA molecules can be modified to form sample nucleic acid derivatives, for example to make them more amenable to adapter ligation. For example, sample DNA molecules can be blunt ended, nucleotides can be added to sample DNA molecules or blunted-ended derivative therefrom, and/or phosphate moieties can be added or removed from the ends of sample DNA molecules or derivatives thereof. In some embodiments, prior to ligation, sample DNA molecules may be blunt ended, and then a single adenosine base can be added to the 3′ end. Prior to ligation the DNA may be cleaved using a restriction enzyme or some other cleavage method. During ligation the 3′ adenosine of the sample fragments and the complementary 3′ thymidine overhang of an adapter can enhance ligation efficiency. Typically, in illustrative embodiments, adapter ligation is performed using a T4 ligase.

In illustrative embodiments, adapters containing one or more universal priming sequences are utilized in methods herein. In some embodiments, the adapters are Y adapters, for example in illustrative methods in which target region amplicons are sequenced using NGS. In some embodiments, the adapters each comprises a universal priming site. Typically, the adapters do not include any MSRE recognition site of any MSRE used in the method.

In some embodiments, the adapters each further comprises a sample barcode. Thus, multiple samples can be analyzed in the same sequencing reaction. The sample barcode can be used to process data according to the sample from which the data was generated.

In some embodiments, the adapters each further comprises a molecular barcode. In some embodiments, the number of adapters having different molecular barcodes is between 10 to 1,000, and wherein the ratio of the total number of sample nucleic acid or cfDNA molecules to the number of different molecular barcodes in the ligation reaction is at least 1,000:1. The number of different molecular barcodes in the ligation reaction, in certain embodiments, ranges from 10 to 50, 10 to 100, 50 to 200, 100 to 300, 200 to 500, 300 to 600, 500 to 700, 600 to 800 or 700 to 1,000. In some embodiments, there are at least 1, 10, 20, 30, 40, 50, or at least 100; 200, 300, 400, 500, 600, 700, 800, 900, or 1000 different molecular barcodes in the ligation reaction. In some embodiments, the ratio of the total number of sample nucleic acid or cfDNA molecules to the number of different molecular barcodes in the ligation reaction is at least 10,000:1. In some embodiments, the ratio of the total number of sample nucleic acid or cfDNA molecules to the number of different molecular barcodes in the ligation reaction ranges from 50,000:1 to 50:1, from 25,000:1 to 100:1, from 10,000:1 to 100:1, from 10:000:1 to 8,000:1 to 500:1, from 5,000:1 to 200:1, from 10,000:1 to 50:1. In some embodiments, the methods disclosed herein result in at least 100; 200; 500; 750; 1,000; 2,000; 5,000; 7,500; 10,000; 20,000; 25,000; 30,000; 40,000; 50,000 different molecular barcodes to each one sample nucleic acid or cfDNA molecules.

### Methylation-Sensitive Restriction Enzymes (MSREs)

Methods herein typically include contacting sample DNA molecules, or nucleic acid derivatives generated therefrom, with one or more MSREs. MSREs are useful in analyzing the methylation status of cytosine residues in CpG sites. As the name implies, these enzymes are not able to cleave their palindromic target sites when the cytosine residues are methylated. The size of the MSRE targets sites range from 4 bp up to 76 bp, but typically are in the 4 to 8 bp range.

In one aspect, methods herein typically include contacting sample DNA molecules, or nucleic acid derivatives generated therefrom, with one or more MSREs. MSREs selectively cleave a sample nucleic acid when the MSRE recognition site is unmethylated, but not when the MSRE recognition site is methylated. An “isoschizomer” of an MSRE is a restriction enzyme that recognizes the same recognition site as a methylation sensitive restriction enzyme but cleaves both methylated CGs and unmethylated CGs. Isoschizomer of the selected MSREs may be used in control reactions. Non-limiting examples of methylation sensitive restriction enzyme include, and thus in some embodiments, the one or more MSREs can include, AatII, Acc65I, AccI, AciI, AclI, AfeI, AgeI, AgeI-HF®, AhdI, AleI-v2, ApaI, ApaLI ApeKI, AscI, AsiSI, AvaI, AvaII, BaeI, BanI, BbvCI, BceAI, BcgI, BcoDI, BfuAI, BglI, BmgBI, BsaAI, BsaBI, BsaHI, BsaI-HF®v2, BseYI, BsiE, BsiWI, BsiWI-HF®, BslI, BsmAI, BsmBI-v2, BsmFI, BspDI, BspEI, BsrBI, BsrFI-v2, BssHII, BstAPI, BstBI, BstUI, BstZ17I-HF®, BtgZI, Cac8I, ClaI, DpnI, DraIII-HF®, DrdI, EaeI, EagI-HF®, EarI, EciI, Eco53kI, EcoRI, EcoRI-HF®, EcoRV, EcoRV-HF®, Esp3I, FauI, Fnu4HI, FokI, FseI, FspI, HaeII, HgaI, HhaI, HinPII, HincII, HinfI, HpaI, HpaII, Hpy166II, Hpy188III, Hpy99I, HpyAV, HpyCH4IV, KasI, MboI, MluI, MluI-HF®, MmeI, MspA1I, MwoI, NaeI, NarI, NciI, NgoMIV, NheI-HF®, NlaIV, NotI, NotI-HF®, NruI, NruI-HF®, Nt.BbvCI, Nt.BsmAI, Nt.CviPII, PaeR7I, PaqCI, PleI, PluTI, PmeI, PmlI, PshAI, PspOMI, PspXI, PvuI, Pvul-HF®, RsaI, RsrII, SacI-HF®, SacII, SalI, SalI-HF®, Sau3AI, Sau96I, ScrFI, SfaNI, SfiI, SfoI, SgrAI, SmaI, SnaBI, SrfI, StyD4I, TfiI, TseI, TspMI, XhoI, XmaI, and/or ZraI. In illustrative embodiments, the one or more MSREs can include HhaI, HpaII, BstUI, and/or HpyCH4IV.

In any of the aspects and embodiments disclosed herein, one or more MSREs can be replaced with one or more methylation dependent restriction enzymes (MDREs). MDREs selectively cleave a sample nucleic acid when one or more nucleotides in the MDRE recognition site is methylated. A skilled artisan will understand how to modify the methods provided herein to include MDREs, or to replace elements recited as MSREs with MDREs. Thus, in some embodiments, one or more MDREs can be used instead of or in the absence of MSREs. In some embodiments, one or more MDREs can be used in combination with one or more MSREs. In some embodiments, the one or more MDREs can be AbaSI, AoxI, BisI, BlsI, DpnI, FspEI, GlaI, GluI, KroI, LpnPI, MalI, MspJI, MteI, PcsI, PkrI, or SgeI.

The MSRE (or MDRE) can be selected based on differentially methylated CpG sites in target DNA molecules, such as tumors or ctDNA from specific cancer targets, or a diverse spectrum of tumors. Further criteria for selection may include low background methylation in normal tissues, size and number of cleavage fragments, number of base pairs of recognition sequence, whether the cleavage results in blunt vs. tailed end fragments, and whether the enzymes have the same or similar reaction conditions such that the contacting step can be done under the same set of conditions and/or in a single reaction.

In some embodiments, more than one, a plurality, or a set of MSREs (and/or MDREs) can be used to contact target DNA molecules comprising one or more CpG sites of interest. The number of selected MSREs in certain embodiments is at least 1, 2, 3, 4, 5, 6, 7, 8, 9, 10, or 20, 25, 50 or 100. In some embodiments, the number of MSREs range from 1 to 2, 3, 4, 5, 6, 7, 8, 9, 10, or 20, 25, 50 or 100; from 2 to 3, 4, 5, 6, 7, 8, 9, 10, or 20, 25, 50 or 100; or from 5 to 6, 7, 8, 9, 10, or 20, 25, 50 or 100. In some embodiments, the number of MSREs range from 1 to 10, 1 to 5, from 2 to 5, from 3 to 5, from 4 to 5, 3 to 7, from 5 to 10, from 6 to 10, or from 7 to 10. Thus, criteria such as target site, target site methylation status, number of base pairs in tailed end fragments, reaction conditions including but not limited to buffer conditions, incubation time and temperature for optimal activity, as well as deactivation time and temperature for each MSRE can be used for selecting the plurality or set of MSREs and/or MDREs to include in the method. As activity measured by units is specific to each enzyme, the target DNA molecules can be contacted with from 1 to 5 Units (U) of each MSREs in the reaction sample. In some embodiments, the target DNA molecules are contacted with from 1 to 5 U, from 1.5 to 4 U, from 2 to 3 U, from 2.5 to 4 U, or from 3 to 5 U of each MSRE. In some embodiments, one or more of the MSRE is selected from HpaII, SalI, BbeI, NotI, SmaI, XmaI, MboI, BstUI, BstBI, ClaI, MluI, NaeI, NarI, PvuI, SacII, HpyCH41V, HhaI, and combinations thereof. In exemplary embodiments, the one or more MSREs is selected from one or more of HpaII, HhaI, HpyCH41V, and BstU1. In some embodiments, the one or more MSREs are selected from one or more of HpaII, HhaI, HpyCH41V, or BstU1. In some embodiments of the methods as described herein, the contacting comprises contacting with two or more MSREs. In some embodiments, the contacting comprises contacting with three or more MSREs. In some embodiments, the contacting comprises contacting with four or more MSREs. In some embodiments, the contacting comprises contacting with the two or more MSREs in a single reaction.

In some embodiments, MSREs and/or MDREs are included that are able to cleave the MSRE sites and/or MDRE sites in the same cleavage buffer conditions. In some embodiments, the cleavage buffer conditions include 5-500 mM potassium acetate, for example 25-100 mM potassium acetate. In some embodiments, the cleavage buffer conditions include 2-200 mM Tris-acetate, for example 10-40 mM Tris-acetate. In some embodiments, the cleavage buffer conditions include 1-100 mM magnesium acetate, for example 5-20 mM magnesium acetate. In some embodiments, the cleavage buffer conditions include 10-1000 μg/ml recombinant albumin, for example 50-200 g/ml recombinant albumin. In some embodiments, the pH is between 6.9 and 8.9 at 25° C., for example, between 7.4 and 8.4, 7.5 and 8.3, 7.6 and 8.2, 7.7 and 8.1, or 7.8 and 8, or about 7.9 at 25° C. In some embodiments, the cleavage buffer conditions include 1-100 mM bis-tris-propane-HCl, for example 5-20 mM bis-tris-propane-HCl. In some embodiments, the cleavage buffer conditions include 1-100 mM MgCl₂, for example 5-20 mM MgCl₂. In some embodiments, the pH is between 6 and 8 at 25° C., for example, between 6.5 and 7.5, 6.6 and 7.4, 6.7 and 7.3, 6.8 and 7.2, or 6.9 and 7.1, or about 7.0 at 25° C. In some embodiments, the cleavage buffer conditions include 5-500 mM NaCl, for example 25-100 mM NaCl. In some embodiments, the cleavage buffer conditions include 1-100 mM Tris-HCl, for example 5-20 mM Tris-HCl. In some embodiments, the cleavage buffer conditions include 10-1000 mM NaCl, for example 50-200 mM NaCl. In some embodiments, the cleavage buffer conditions include 5-500 mM Tris-HCl, for example 25-100 mM Tris-HCl. In some embodiments, the cleavage buffer conditions include 25-100 mM potassium acetate, 10-40 mM Tris-acetate, 5-20 mM magnesium acetate, and 50-200 μg/ml recombinant albumin, and the pH is between 7.6 and 8.2 at 25° C. In some embodiments, the cleavage buffer conditions include 5-20 mM bis-tris-propane-HCl, 5-20 mM MgCl₂, and 50-200 g/ml recombinant albumin, and the pH is between 6.7 and 7.3 at 25° C. In some embodiments, the cleavage buffer conditions include 25-100 mM NaCl, 5-20 mM Tris-HCl, 5-20 mM MgCl₂, and 50-200 g/ml recombinant albumin, and the pH is between 7.6 and 8.2 at 25° C. In some embodiments, the cleavage buffer conditions include 50-200 mM NaCl 25-100 mM Tris-HCl, 5-20 mM MgCl₂, and 50-200 g/ml recombinant albumin, and the pH is between 7.6 and 8.2 at 25° C.

### Amplifications

Methods in some aspects herein include performing one or, in some embodiments, two or more amplifications. Such amplifications in certain illustrative embodiments include at least one targeted amplification wherein at least one primer and in certain embodiments both primers of a primer pair, one or more primer pairs, or a set of primer pairs used for the amplification are each designed to bind to a specific nucleic acid sequence at or near, typically within, a genomic region of interest (i.e. are target-specific primers) to generate target region amplicons. In some embodiments, methods herein include one or more universal amplifications.

A number of amplification technologies can be used with methods herein. For example, such amplification can be an isothermal amplification (e.g. recombinase polymerase amplification (RPA) (Kersting et al. 2014 Microchim Acta 181 (13-14), 1715-1723, (incorporated by reference in its entirety)), a ligase-based amplification, PCR, or a combination thereof (e.g. ligation-mediated PCR). In some illustrative embodiments, the targeted amplification is a targeted PCR(s) that is performed using a PCR reaction mixture that includes one primer pair, or in illustrative embodiments a set of primer pairs, and MSRE-exposed adapted DNA molecules (e.g. MSRE-exposed adapted cfDNA), or universally amplified MSRE-exposed adapted DNA molecules generated therefrom.

FIG. 4 illustrates various potential positions of primer binding sites on a DNA molecule that is subjected to exemplary methods herein. In the example illustrated in FIG. 4, the adapted DNA molecule comprises a sample DNA region flanked by two Y adapters each with a 5′ adapter strand (350) and a 3′ adapter strand (355). The sample DNA region in this example includes a target region (shown in hashed light grey) having two CpG sites (shown as encircled “mC”) within the target region and one CpG site near the target region, all of which, in this example, are methylated. Typically, at least one primer of a primer pair used for targeted amplification herein, is a target-specific primer designed to bind to a specific nucleic acid sequence in or near, typically within, a genomic region of interest, which in illustrative examples can be genomic regions where epigenetic changes, such as changes in DNA methylation, are associated with or indicative of formation or presence of cancer, and can, for example, include promoter regions of tumor suppressor genes, and in some embodiments DNA methylation markers for a specific cancer type. A target-specific primer (320, 380, 325, 385) can be designed to bind to any sequence within or near a target region for amplification of the target region or a portion of the target region. In some embodiments, a target-specific primer can be designed to bind to a sequence that includes a CpG site covered by an MSRE recognition sequence. In other embodiments, a target-specific primer can be designed to bind to a sequence that is upstream or downstream to one or more CpG sites covered by one or more MSRE recognition sequences. One of the advantages of the methods described herein is increased flexibility in primer/probe design for targeted amplification or enrichment. In some embodiments, one primer of the one or more primer pairs or the set of primer pairs in the reaction mixture used for a targeted amplification is a universal primer (310, 315) and binds to a primer binding site on at least one of the adapters. Thus, for example, in such embodiments a universal primer (310) that binds an adapter primer binding site can be used for an amplification reaction along with a target-specific primer (325 or 385) that binds a primer binding site on a sample DNA region.

Target-specific primers typically define the ends of target region amplicons, which typically encompass at least a portion of the target region. In some embodiments, a PCR can be performed using two target-specific primers 320, 385. The target region amplicon in such embodiment would extend from the sample DNA region bound by target-specific primer 320 on a 5′ end to the sample DNA region bound by primer 385 on the 3′ end. In some embodiments, a PCR can be performed using a universal primer 310 and a target-specific primer 385. The target region amplicon in such embodiment would extend from the sample DNA region bound by target-specific primer 385 on a 3′ end of one strand to the end of the sample DNA fragment on the 5′ end of that strand.

In illustrative embodiments, target-specific primers can be designed to generate target region amplicons that encompass one or more CpG sites in a target region covered by one or more MSRE recognition sequences. The one or more MSRE recognition sites in such embodiments can be located within or near a prospective target region amplicon or overlap with a target-specific primer binding site.

In some embodiments, a sample DNA fragment may include an MSRE site located outside a prospective target region amplicon (e.g. MSRE recognition site 370, in relation to prospective target region amplicon to be generated using primers 320, 385). In such embodiments, a universal preamplification of the adapted DNA molecules can selectively enrich sample DNA fragments in which all MSRE recognition sites are methylated, including off-amplicon MSRE sites such as 370. Sample DNA fragments in which one or more off-amplicon MSRE sites are not methylated will typically be cleaved by one or more MSREs and will typically not be amplified using the universal primers that bind priming sites on the adapters, even if all the MSRE sites within the prospective target region amplicon are methylated. In cancer cells, CpG sites in CpG islands at regulatory regions are predominantly co-methylated. Thus, the methods disclosed herein offer both increased flexibility in primer or probe placement/design and better specificity in detecting DNA from cancer cells.

In some embodiments, both primers of at least one primer pair of the one or more primer pairs, or of the set of primer pairs, are target-specific primers. Thus, for example, primer 320 can be used with primer 325 or primer 385. In some embodiments, both primers of a plurality of the one or more primer pairs, or of each primer pair of the set of primer pairs, are target-specific primers. Such target-specific primer binding sites, in some embodiments, can overlap with target CpG sites covered by one or more MSRE recognition sites.

As discussed above, in some methods herein, a universal amplification(s) can be performed before the targeted amplification(s). Such universal amplification can be performed for example using a universal primer pair (310, 315) that binds primer binding sites in the adapter. Such universal amplifications (e.g. universal PCRs) can be included in certain illustrative embodiments wherein sample DNA molecules are expected to include one or more MSRE recognition sites located outside a prospective target region amplicon, as discussed above. Thus, in some embodiments, the methods herein include performing a universal PCR using the plurality of adapted DNA molecules or the plurality of adapted cfDNA, and a universal PCR primer pair comprising primers designed to bind universal primer binding sequences on the adapters, to generate amplified MSRE-exposed, adapted DNA molecules or MSRE-exposed, adapted cfDNA, before performing one or more targeted PCRs, or a set of targeted PCRs using the amplified MSRE-exposed, adapted DNA molecules or the amplified MSRE-exposed, adapted cfDNA.

In some embodiments, at least one primer of at least one primer pair, or a plurality of primer pairs, or each primer pair of the one or more primer pairs, or of the set of primer pairs, is a target-specific primer. Methods herein can include primer pairs designed to bind to uncleaved versions of the adapted DNA molecules or the adapted cfDNA, but not cleaved adapted DNA molecules.

In some embodiments of any aspects herein, one or more of the target regions, or set of the target regions, comprise one or more MSRE recognition sites overlapping a primer binding site of the one or more, or set of, primer binding sites.

The one or more primer pairs in illustrative embodiments is a set of primer pairs. In some embodiments, the set of primer pairs is a set of between 2 and 1,000, 2 and 500, 2 and 250, 2 and 200, 2 and 150, 2 and 100, 2 and 50 or 2 and 10 primer pairs, or between 5 and 1,000, 5 and 500, 5 and 250, 5 and 200, 5 and 150, 5 and 100, 5 and 50 or 5 and 10 primer pairs, or between 50 and 250 or between 100 and 200 primer pars.

In some embodiments, at least one of the primer pairs comprises a universal primer and a target-specific primer. In some embodiments, at least one of the primer pairs comprises two target-specific primers. In some embodiments, at least one of the primers comprises a sequencing tag. In some embodiments, at least one of the primers comprises a sample index. In some embodiments, at least one of the primers comprises biotin modification. In some embodiments, performing a PCR further comprises using primers comprising a sequencing tag. In some embodiments, performing a PCR further comprises using primers comprising a sample index. In some embodiments, the primers of the primer pairs are probe-dependent primers and the amplification is a target capture polymerase chain reaction. In some embodiments, the target regions each comprises a set of two or more MSRE recognition sites differentially methylated in one or more cancers. In some embodiments, the set of loci comprises two or more loci that are differentially methylated in a different type of cancer from each other. In some embodiments, the target regions each comprises three or more MSRE recognition sites differentially methylated in a cancer. In some embodiments, the MSRE recognition sites in one group of the target regions are methylated in one type of cancer, and the MSRE recognition sites in another group of the target regions are methylated in a different type of cancer.

Some of the adapted DNA molecules, in illustrative embodiments, MSRE-exposed, adapted DNA molecules, comprise one or more target regions, wherein at least some of the one or more target regions comprise one or more MSRE recognition sites. In some embodiments, after a step of contacting adapted DNA molecules with one or more MSREs, and in illustrative embodiments further after a step of universal amplification, PCR is performed in methods herein using a plurality of MSRE-exposed, adapted DNA molecules, or a plurality of pre-amplified MSRE-exposed, adapted DNA molecules, and one or more primer pairs designed to amplify one or more target regions or portions thereof in the MSRE-exposed, adapted DNA molecules or copies thereof, and wherein target region amplicons are generated if all of the MSRE recognition sites within or near a prospective target region amplicon are methylated.

In some embodiments, the methods herein can further comprise using one or more primer pairs designed to bind primer binding sites on the adapters of the adapted DNA molecules, and thereby designed to amplify the MSRE-exposed, uncleaved adapted DNA molecules or copies thereof. In some embodiments, the MSRE-exposed, adapted DNA molecules or copies thereof comprise one or more MSRE recognition sites outside a prospective target region amplicon, wherein target region amplicons are generated if all of the MSRE recognitions sites in the MSRE-exposed, adapted DNA molecules, including the one or more MSRE recognition sites outside the prospective target region amplicon, are methylated. In some embodiments of the methods that comprise using one or more primer pairs designed to bind primer binding sites on the adapters of the MSRE-exposed, adapted DNA molecules, the one or more primer pairs bind primer binding sites on only one of the adapters of each one of the MSRE-exposed, adapted DNA molecules, and thereby designed to amplify the MSRE-exposed, adapted DNA molecules or copies thereof.

In some embodiments, a PCR is performed in methods herein using one or more primer pairs designed to amplify one or more target regions in the MSRE-exposed, adapted DNA molecules, and thereby generating target region amplicons. In some embodiments, both primers of a plurality of the one or more primer pairs, or of the set of primer pairs, are target-specific primers. In some embodiments, each primer of the one or more primer pairs, or of the set of primer pairs, is a target-specific primer. In some embodiments, both primers of at least one primer pair of the one or more primer pairs, or of the set of primer pairs, are target-specific primers.

In some embodiments, a PCR is performed in methods herein using one or more primer pairs designed to bind primer binding sites on the adapters of the MSRE-exposed, adapted DNA molecules, and thereby designed to amplify the MSRE-exposed, uncleaved adapted DNA molecules. In some embodiments, at least one primer of at least one primer pair of the one or more primer pairs, or of the set of primer pairs, is designed to bind to a primer binding site on one of the adapters. In some embodiments, at least one primer of a plurality of primer pairs of the one or more primer pairs, or of the set of primer pairs, is designed to bind to a primer binding site on one of the adapters. In some embodiments, at least one primer of each primer pair of the one or more primer pairs, or of the set of primer pairs, is designed to bind to a primer binding site on one of the adapters. In some embodiments, at least one of the primer binding sites of a primer pair can include one or more MSRE sites. In some embodiments, one of the primer binding sites of a primer pair can include one or more MSRE sites. In some embodiments, both of the primer binding sites of a primer pair can include one or more MSRE sites. In some embodiments, neither of the primer binding sites of a primer pair include an MSRE site. In some embodiments, an amplicon generated from performing PCR with a primer pair includes one or more MSRE sites, for example 1, 2, 3, 4, 5, 6, 7, 8, 9, 10, 11, 12, 13, 14, 15, 16, 17, 18, 19, or 20 MSRE sites. In some embodiments, an amplicon generated from performing PCR with a primer pair includes no MSRE sites. In some embodiments, the one or more MSRE sites are within 5, 10, 15, 20, 25, 30, 35, 40, 45, 50, 55, 60, 65, 70, 75, 80, 85, 90, 95, or 100 nucleotides of a primer binding site. In some embodiments, one or more of the one or more MSRE sites overlap with a primer binding site.

In some embodiments, one or both of the primer binding sites of a primer pair can include at least a portion of one of the adapter sequences. In some embodiments, one of the primer binding sites of a primer pair can include at least a portion of the adapter sequences. In some embodiments, both of the primer binding sites of a primer pair can include at least a portion of the adapter sequences. In some embodiments, neither of the primer binding sites of a primer pair include any of the adapter sequences.

In some embodiments, a plurality of the adapted DNA molecules having one or more MSRE recognition sites, or the adapted cfDNA having one or more MSRE recognition sites, comprise one or more target regions each comprising two or more MSRE recognition sites. In some embodiments, a prospective target region amplicon includes 1, 2, 3, 4, 5, 6, 7, 8, 9, or 10 MSRE sites. In some embodiments, the prospective target region amplicon includes between 1 and 20 MSRE sites, for example, between 1 and 15, 1 and 10, 1 and 8, 1 and 6, 1 and 5, 1 and 4, 1 and 3, or 1 and 2 MSRE sites, or between 2 and 15, 2 and 10, 2 and 8, 2 and 6, 2 and 5, 2 and 4, or 2 and 3 MSRE sites.

Methods as described herein, in some embodiments, can include multiple amplification cycles (e.g. multiple PCR temperature cycles), and in some embodiments can include several sequential PCR reactions performed during the same set of temperature cycles. In some embodiments, amplification cycles can include at least 2, 3, 4, 5, 6, 7, 8, 9, 10, 11, 12, 13, 14, 15, 16, 17, 18, 19, or 20 cycles. In some embodiments, amplification cycles can include at least 7, 8, 9, or 10 cycles. In illustrative embodiments, amplification cycles can include at least 11, 12, 13, 14, 15, 16, or 17 cycles.

Typically, in embodiments described herein, PCR amplification is performed by adding a PCR reaction mixture to the DNA template (e.g., MSRE-exposed, adapted sample DNA or cfDNA) followed by addition of a polymerase enzyme, and then amplified through multiple amplification cycles. In some embodiments, the PCR reaction mixture contains one or more primer pairs, deoxynucleotides (dNTPs), PCR reaction buffer, and deionized water. In some embodiments, the dNTPs can comprise a mixture of dATP, dCTP, dGTP and dTTP. In some embodiments, the final concentration of each dNTP in the reaction mixture can range from 0.05 mM to 0.5 mM dNTPs, for example, from 0.05 mM to 0.5 mM, 0.05 mM to 0.1 mM, 0.05 mM to 0.15 mM, 0.05 to 0.2 mM, 0.05 mM to 0.25 mM, 0.05 mM to 0.3 mM, 0.05 mM to 0.35 mM, 0.05 to 0.4 mM 0.05 mM to 0.45 mM, from 0.1 mM to 0.5 mM, 0.15 mM to 0.5 mM, or 0.2 mM to 0.5 mM, 0.25 to 0.5 mM, 0.3 mM to 0.5 mM, 0.35 mM to 0.5 mM, 0.4 mM 0.5 mM, or from 0.45 mM to 0.5 mM. In illustrative embodiments, the final concentration of each dNTP in the reaction mixture is between 0.15 mM and 0.25 mM. In some embodiments, the final concentration of each dNTP in the reaction mixture is 0.2 mM.

PCR buffer solution creates a suitable environment for the polymerase chain reaction and can contain many different components, including magnesium chloride (MgCl₂), potassium chloride (KCl), dimethyl sulfoxide (DMSO), and glycerin or bovine serum albumin (BSA). In some embodiments, the concentration of KCl can be between 25 and 50 mM, between 25 and 75 mM, between 25 and 100 mM, between 30 and 100 mM, between 50 and 100 mM, or between 70 and 100 mM. In some embodiments, the MgCl2 concentration can be in the range of 0.5 mM to 5 mM, between 0.5 mM to 4.5 mM, 0.5 to 4.0 mM, 0.5 to 3.5 mM, 0.5 to 3.0 mM, 0.5 to 2.5 mM, 0.5 to 2.0 mM, 0.5 to 1.5 mM, 1.0 to 5 mM, 1.5 to 5 mM, 2.0 to 5 mM, 2.5 to 5 mM, 3.0 to 5 mM, 3.5 to 5 mM, 4.0 to 5 mM, or 4.5 to 5 mM. In some embodiments, the concentration of MgCl2 is 2.0 mM.

In illustrative embodiments, the buffer solution is a Q5® Reaction Buffer (B9027S, New England Biolabs, Inc.). In some embodiments, the reaction buffer is Standard Taq Reaction Buffer (B9014S, New England Biolabs, Inc). In some embodiments, the reaction buffer is a Standard Taq (Mg-free) Reaction Buffer (B9015S, New England Biolabs, Inc.).

In some embodiments, a DNA polymerase is used to produce DNA amplicons using DNA as a template. In some embodiments, the polymerase is a Q5® DNA Polymerase, such as Q5® High-Fidelity DNA Polymerase (M0491S, New England BioLabs, Inc.) or Q5® Hot Start High-Fidelity DNA Polymerase (M0493S, New England BioLabs, Inc.). Q5® High-Fidelity DNA polymerase is a high-fidelity, thermostable, DNA polymerase with 3′4 5′ exonuclease activity, fused to a processivity-enhancing Sso7d domain. Q5® High-Fidelity DNA polymerase lacks 5′→3′exonuclease activity and strand displacement activity.

In some embodiments, the polymerase is a T4 DNA polymerase (M0203S, New England BioLabs, Inc.). T4 DNA Polymerase catalyzes the synthesis of DNA in the 5′→3′ direction and requires the presence of template and primer. This enzyme has a 3′→5′ exonuclease activity which is much more active than that found in DNA Polymerase I. T4 DNA polymerase lacks 5′→3′ exonuclease activity and strand displacement activity.

In some embodiments of any of the aspects herein, the length of the primers can be between 10 to 100 nucleotides, such as between 10 to 75 nucleotides, 10 to 40 nucleotides, 10 to 35 nucleotides, 10 to 30 nucleotides, 10 to 20 nucleotides, 15 to 100 nucleotides, 20 to 100 nucleotides, from 25 to 100 nucleotides, from 30 to 100 nucleotides from 35 to 100 nucleotides, from 40 to 100 nucleotides, from 45 to 100 nucleotides, from 50 to 100 nucleotides, from 55 to 100 nucleotides, from 60 to 100 nucleotides, from 65 to 100 nucleotides, from 70 to 100 nucleotides, or from 75 to 100 nucleotides. In some embodiments, the range of the length of the primers is between 5 to 50 nucleotides, such as 5 to 40 nucleotides, 5 to 20 nucleotides, or 5 to 10 nucleotides. In some embodiments, the primers are between 5 and 50 bp in length, between 10 and 40 bp in length, between 15 and 30 bp in length, between 15 and 25 bp in length, between 20 and 40 bp in length, between 25 and 50 bp in length, or between 30 and 50 bp in length. In some embodiments, the primers are between 25 and 100 bp in length, between 35 and 100 bp in length, between 45 and 100 bp in length, between 55 and 100 bp in length, between 65 and 100 bp in length, or between 75 and 100 bp in length.

In some embodiments of any of the aspects or embodiments herein, the number of primer pairs can range from 1 to 100,000 primer pairs that each bind to one or more primer binding sequences. In some embodiments, the primer pairs are a part of a set of primer pairs. In some embodiments, the set of primers range from 2 to 100,000, from 2 to 10,000, from 2 to 1,000, from 2 to 100, from 2 to 50, from 10 to 100, from 50 to 100, from 100 to 200, from 100 to 500, from 100 to 1,000, from 100 to 10,000, from 100 to 100,000, from 1,000 to 100,00, or from 10,000 to 100,000 primer pairs. In some embodiments, the number of primer pairs can range from 10 to 10,000, 10 to 1,000, 10 to 100, 10 to 50, 10 to 40, 10 to 30, 15 to 30, or 15 to 25 primer pairs.

In some embodiments, PCR is used to generate very short amplicons. cfDNA (such as fetal cfDNA in maternal serum or necrotically- or apoptotically-released cancer cfDNA) is highly fragmented. For fetal cfDNA, the fragment sizes are distributed in approximately a Gaussian fashion with a mean of 160 bp, a standard deviation of 15 bp, a minimum size of about 100 bp, and a maximum size of about 220 bp. Methylation site(s) of interest may occupy any position from the start to the end among the various fragments originating from a particular locus. Because cfDNA fragments are short, the likelihood of both primer sites being present the likelihood of a fragment of length L comprising both the forward and reverse primers sites is the ratio of the length of the amplicon to the length of the fragment. Under ideal conditions, assays in which the amplicon is 45, 50, 55, 60, 65, or 70 bp will successfully amplify from 72%, 69%, 66%, 63%, 59%, or 56%, respectively, of available template fragment molecules. Thus, in some embodiments target amplicons generated in method herein are between 40 and 100, 40 and 75, or 45 and 70 bp in length. In certain embodiments that relate most preferably to cfDNA from samples of individuals suspected of having cancer, the cfDNA is amplified using primers that yield a maximum amplicon length of 85, 80, 75 or 70 bp, and in certain preferred embodiments 75 bp, and that have a melting temperature between 5° and 65° C., and in certain preferred embodiments, between 54-60.5° C. The amplicon length is the distance between the 5-prime ends of the forward and reverse priming sites. Amplicon length that is shorter than typically used by those known in the art may result in more efficient measurements of the desired methylation sites by only requiring short sequence reads. In an embodiment, a substantial fraction of the amplicons are between 25 on the low end of the range, and 100 bp, 90 bp, 80 bp, 70 bp, 65 bp, 60 bp, 55 bp, 50 bp, or 45 bp on the high end of the range.

### Probe-Dependent Primers

In some embodiments, one or more primers herein are probe-dependent primers. Probe-dependent primers (PDPs) have been disclosed (Pel, et al. “Rapid and highly-specific generation of targeted DNA sequencing libraries enabled by linking capture probes with universal primers” PLoS ONE 13(12):e0208283 (2018); WO 2020/039261 “Linked target capture and ligation”, which are hereby incorporated by reference in their entirety). Such embodiments can be considered LTC methods. Briefly, in an LTC method, PDPs are designed to incorporate non-extendable capture probes linked 5′ to 5′ with a primer. Multiple linker types are possible as discussed below. Typically, probes of PDPs can be between 30 to 70 nucleotides in length, and include or comprise a 3′ inverted dT base to inhibit polymerase extension. In some embodiments, probes are designed to cover the desired region with zero gap between forward and reverse probes. In some embodiments, the probes are between 20 and 100 nucleotides in length. In some embodiments, the size of the probe can be between 20 and 40 nucleotides, between 30 and 50 nucleotides, between 40 and 60, between 50 and 70 between 60 and 80, between 70 and 90, 80 and 100, 90 and 110, 100 and 120 nucleotides in length. In some embodiments, at least one of the probes of a PDP pair comprises a sample index.

In PDPs, forward and reverse probes can be designed to bind to nucleic acid sequences within or near a genomic region of interest on a sample DNA molecule to enrich nucleic acid molecules comprising the genomic region of interest or copies thereof. In some embodiments, at least one of the probe binding regions can include one or more MSRE sites. In some embodiments, one of the probe binding regions can include one or more MSRE sites. In some embodiments, both of the probe binding sites of the probe binding regions can include one or more MSRE sites. In some embodiments, neither of the probe binding sites of the probe binding region comprises an MSRE site.

Typically, the primer portion of a PDP is a universal primer designed to bind to a universal primer site on the ligated adapter. In some embodiments, the PDP is designed with a sequencer binding sequence, such as an Illumina flow cell binding sequence, incorporated therein. In some embodiments, the sequencer flow cell binding sequence is between the probe and universal primer, and adjacent to the primer. Linked primers of the invention may also include sequencing tags to ensure that all cluster reads originate from the same linked template molecule. The lengths of the primers can be extended or shortened at the 5′ end or the 3′ end to produce primers with desired melting temperatures. Also, the annealing position of each primer pair can be designed such that the sequence and length of the primer pairs yield the desired melting temperature. In illustrative embodiments, the primer is a low melting temperature universal primer complementary to a portion of the ligated adapter.

The primer can be tailed or untailed depending on the specific requirements. In some embodiments, the universal primer comprises an A tail. In some embodiments, the universal primer is blunt ended. The length of the primers of the PDP can range from 5 to 40 nucleotides in length. In certain embodiments, the PDP primers are between 10 and 25 nucleotides long. In embodiments, the primers of the PDP can range from 5 to 15 nucleotides, from 10 to 25 nucleotides, from 15 to 35 nucleotides, or from 25 to 40 nucleotides in length.

Typically, probe dependent primers comprise a linker between the probe and the primer. Probe and primer portions of the PDP are typically linked by a polyethylene glycol derivative, an oligosaccharide, a lipid, a hydrocarbon, a polymer, or a protein. In some embodiments, the linker is a PEG molecule, or derivative thereof. In some embodiments, the linker is an oligosaccharide. In some embodiments, the linker is a lipid. In some embodiments, the linker is a hydrocarbon. In some embodiments, the linker is a polymer. In some embodiments, the linker is a protein, or portion thereof.

### Hybrid Capture

As illustrated in FIG. 1, in some embodiments, one or more selective enrichment steps (152) can be used to enrich DNA molecules comprising one or more target regions. For example, the selective enrichment technique can involve fragment capture by hybridization (i.e. hybrid capture). Although any hybrid capture method can be used to perform methods herein that include a selective enrichment step, in some embodiments, a method of the present disclosure may involve using any of the hybrid capture methods disclosed herein to selectively enrich DNA, for example, cfDNA. Such selective enrichment step can follow an amplification step, typically follows a universal pre-amplification step, sometimes immediately following a universal pre-amplification step. Furthermore, one or more selective enrichment steps can occur before one or more downstream universal or targeted amplification steps. For example, a method herein can include performing a universal amplification wherein at least a plurality of MSRE-exposed, uncleaved, adapted cfDNA are amplified to generate amplicons, followed by performing a hybrid capture using hybrid capture probes to selectively enrich amplified DNA molecules comprising one or more target regions. As another example, a method herein can include performing a targeted amplification to amplify one or more target regions from a plurality of MSRE-exposed, uncleaved adapted cfDNA molecules or copies thereof, wherein target region amplicons are formed if the one or more target regions are present in the MSRE-exposed, uncleaved adapted cfDNA molecules, followed by performing hybrid capture method using hybrid capture probes to selectively enrich one or more target region amplicons.

In one aspect, provided herein is a method for preparing DNA molecules, in illustrative embodiments, cfDNA molecules, useful for determining a methylation status of selected regions of the DNA molecules, comprising
a) ligating adapters to cfDNA obtained or derived from a first liquid sample, thereby forming a plurality of adapted cfDNA comprising adapted cfDNA having one or more MSRE recognition sites;
b) contacting the plurality of adapted cfDNA with one or more MSREs, thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA;
c) amplifying at least a plurality of the MSRE-exposed, uncleaved, adapted cfDNA;
d) selectively enriching subsets of amplified MSRE-exposed, uncleaved, adapted cfDNA having one or more target regions using a set of hybrid capture probes, wherein each target region comprises one or more MSRE recognition sites, and wherein each probe of the set is designed to hybridize to one target region; and
e) quantifying an amount for at least some of the enriched subsets by performing a next-generation sequencing reaction on clonally amplified enriched subsets, or amplicons derived therefrom.

In capture by hybridization, hybrid capture oligonucleotide probes complementary to one strand of a specific target DNA sequence, or DNA derived therefrom in a sample, are utilized. The specific target DNA sequence in illustrative embodiments overlaps with, or is found within a target region of a sample DNA molecule such as a cfDNA. Thus, hybrid capture probes when used in methods herein can be designed to bind to a DNA molecule that contains at least one target region or a portion thereof. In some embodiments, the hybrid capture probes can be designed to bind to a target DNA sequence within or overlapping a target region, which in illustrative embodiments contains one or more MSRE recognition sites. In other examples, the hybrid capture probes can be designed to bind to a common region that is flanking but not overlapping the target region and that can be a common region that was added to some, most, almost all or all of the DNA in a sample, or added to all amplicons using a common sequence on at least one primer of a primer pair. In illustrative embodiments, a hybrid capture probe or set thereof, are designed to bind to a target DNA sequence within target region, or set of target regions, respectively.

Hybrid capture probes may be added to a prepared sample and hybridized through a denature-reannealing process to form duplexes of exogenous-endogenous fragments (e.g. hybrid capture probes bound to sample DNA molecules, or DNA derived therefrom). These duplexes may then be physically separated from the sample by various means. In some embodiments, once the hybrid capture probes are removed, the sample DNA molecules, or DNA derived therefrom can be amplified. Some ways to physically remove the hybrid capture probes are by covalently bonding the hybrid capture probes to a solid support, for example a magnetic bead, or a chip. Another way to physically remove the hybrid capture probes is by covalently bonding them to a molecular moiety with a strong affinity for another molecular moiety. An example of such a molecular pair is biotin and streptavidin, such as is used in SURE SELECT (Agilent). Thus, hybrid capture probes, for example that bind to a target DNA sequence within or overlapping a target region of a DNA molecule obtained or derived from a sample, can be covalently attached to a biotin molecule, and after hybridization with sample DNA or DNA derived therefrom, a solid support with streptavidin affixed can be used to pull down the biotinylated hybrid capture probes, which are hybridized to DNA molecules obtained or derived from a sample that include a target region that includes the target DNA sequence recognized by the hybrid capture probes. Thus, in some embodiments, the hybrid capture probes are immobilized, directly or indirectly to a solid support. In some embodiments, the hybrid capture probes include a binding partner, for example biotin.

In some embodiments of any of the aspects herein, the hybrid capture probes can be a part of a set of at least two hybrid capture probes, wherein each hybrid capture probe can be designed to bind to a different target sequence in a target region. In some embodiments, the set includes at least one hybrid capture probe for each target region. In some embodiments, the set includes two or more hybrid capture probes for each target region.

In some embodiments of any of the aspects herein, the hybrid capture probes can have a length in the range of 30 bases to 170 bases, 30 bases to 160 bases, 30 bases to 150 bases, 30 bases to 140 bases, 30 bases to 130 bases, 30 bases to 120 bases, 30 bases to 110 bases, 30 bases to 100 bases, 30 bases to 90 bases, 30 bases to 80 bases, 30 bases to 70 bases, 30 bases to 60 bases, 30 bases to 50 bases, 40 bases to 160 bases, 40 bases to 150 bases, 40 bases to 140 bases, 40 bases to 130 bases, 40 bases to 120 bases, 40 bases to 110 bases, 40 bases to 100 bases, 40 bases to 90 bases, 40 bases to 80 bases, 40 bases to 70 bases, 40 bases to 60 bases, 50 bases to 150 bases, 50 bases to 140 bases, 50 bases to 130 bases, 50 bases to 120 bases, 50 bases to 110 bases, 50 bases to 100 bases, 50 bases to 90 bases, 50 bases to 80 bases, 50 bases to 70 bases, 60 bases to 140 bases, 60 bases to 130 bases, 60 bases to 120 bases, 60 bases to 110 bases, 60 bases to 100 bases, 60 bases to 90 bases, 60 bases to 80 bases, 70 bases to 130 bases, 70 bases to 120 bases, 70 bases to 110 bases, 70 bases to 100 bases, 70 bases to 90 bases, 80 bases to 120 bases, 80 bases to 110 bases, 80 bases to 100 bases, 90 bases to 120 bases, 90 bases to 110 bases, 100 bases to 165 bases, 100 bases to 150 bases, 100 bases to 140 bases, 100 bases to 130 bases, 100 bases to 120 bases, 110 bases to 150 bases, 110 bases to 140 bases, 110 bases to 130 bases, 120 bases to 150 bases, or 130 bases to 160 bases.

If hybrid capture is used upstream of a next-generation sequencing reaction, one way to increase the number of reads that interrogate the position of interest is to decrease the length of the hybrid capture probe, as long as it does not result in bias in the underlying enriched alleles. The length of the hybrid capture probe should be long enough such that two hybrid capture probes designed to bind to two different target DNA sequences within the same target regions hybridize with near equal affinity to the target sequences. In certain embodiments, the use of shorter probes results in a greater chance that the hybrid capture probes bind to DNA molecular fragments from liquid samples, such as cfDNA. Furthermore, in some embodiments hybrid capture probes can be designed to bind to 2 DNA sequences that are separated by 1, 2, 3, 4, 5, 6, 7, 8, 9, 10, 11-20, or more than 20 nucleotides on a target DNA, for example within the same target region. In some embodiments, for each target region, hybrid capture probes can be designed to bind to different DNA sequences within the target region that overlap by 0, 5, 10, 15, 20, 25, 30, 35, 40, 45, 50, 55, 60, 65, 70, 75, 80, 85, 90, 100, 110, 120 or between 5 to 120 nucleotides. Thus, using hybrid capture, DNA molecules that include targeted regions in the DNA sample can be selectively enriched.

### Comparison of 2 or More Samples

Methods herein, in some embodiments, can include comparing 2 or more samples. In some embodiments, methods as described herein can include comparing 3, 4, 5, 6, 7, 8, 9, 10 or more samples. In some embodiments, methods as described herein can include comparing 2 samples, such methods further comprise performing the method on a second sample, and wherein determining the methylation status of the set of target regions in the first sample further comprises comparing the amount of target region amplicons derived from the first sample to the amount of target region amplicons derived from the second sample. In some embodiments, the second sample is from a second subject. In some embodiments, the second sample is derived from a cell line. In some embodiments, the method is performed on the first and second samples simultaneously. In some embodiments, methods as described herein can include comparing 2 samples, such methods further comprises performing the method on a second sample, and wherein determining the methylation status of the set of target DNA molecules in the first sample further comprises comparing the amount of the amplified, methylated target DNA molecules from each of the set of target DNA molecules in the first sample to the amount of the amplified, methylated target DNA molecules from each of the set of target DNA molecules in the second sample. In some embodiments, the first subject is suspected or at risk of having a disease, and wherein the second subject is not suspected or at risk of having the disease. In some embodiments, the second sample is from the first subject. In some embodiments, the second sample is collected at a first timepoint and the first sample is collected at a second timepoint. In some embodiments, the first timepoint precedes the second timepoint. In some embodiments, the determining comprises comparing the amount quantified for the target region amplicons derived from the first sample to an amount quantified for target region amplicons derived from a second sample using the same method. In some embodiments, the method further comprises in addition to a first performance of the method, a second performance of the method performed at the same time as, or a different time than the first performance, wherein the second performance does not comprise the contacting step, and wherein the method further comprises comparing the amount quantified in the first performance to the amount quantified in the second performance to determine the methylation status of the target nucleic acid. In some embodiments, the determining comprises comparing the amount quantified for the target region amplicons to a preset threshold amount.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects can be combined with any other embodiment or aspect, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

Provided herein in one aspect is a method for preparing deoxyribonucleic acid (DNA) molecules useful for determining a methylation status of a genomic region of interest, said method comprising
a) ligating adapters to sample DNA molecules obtained or derived from a first sample from a first subject, thereby forming a plurality of adapted DNA molecules comprising adapted DNA molecules having methylation sensitive restriction enzyme (MSRE) recognition sites;
b) contacting the plurality of adapted DNA molecules with one or more MSREs, thereby generating MSRE-exposed, adapted DNA molecules, wherein at least a first plurality of the MSRE-exposed, adapted DNA molecules having one or more unmethylated MSRE recognition sites are cleaved by at least one of the MSREs, and wherein a second plurality of MSRE-exposed adapted DNA molecules are uncleaved by any of the MSREs, thus forming MSRE-exposed, uncleaved adapted DNA molecules; and
c) performing one or more amplification reactions to amplify one or more target regions from the MSRE-exposed, uncleaved adapted DNA molecules or copies thereof, wherein target region amplicons are formed if the one or more target regions are present in the MSRE-exposed, uncleaved adapted DNA molecules.

In some embodiments, each prospective target region amplicon has one or more MSRE recognition sites. In some embodiments, each prospective target region amplicon has two or more MSRE recognition sites. In some embodiments, at least 10% of the prospective target region amplicons each comprises at least two MSRE recognition sites. In some embodiments, at least 15%, 20%, 25%, 30%, or 35% of the prospective target region amplicons each comprises at least two MSRE recognition sites. In some embodiments, methods as described herein can include at least one prospective target region amplicon comprising at least three, four, five, or six MSRE recognition sites. In some embodiments, methods as described herein can include at least one prospective target region amplicon comprising between 2 and 45 MSRE recognition sites.

In some embodiments, the sample DNA molecules are circulating free DNA (cfDNA). In some embodiments, one or more target region amplicons are generated by one or more polymerase chain reactions (PCRs) using one or more primer pairs, and in illustrative embodiments at least one primer of each of the one or more primer pairs is a target-specific primer. In some embodiments, one or more target region amplicons are generated using one or more capture probes designed to hybridize to one or more target regions. In some embodiments, one or more target region amplicons are generated by one or more linked target capture (LTC) reactions using one or more probe-dependent primer pairs. In some embodiments, the method further comprises detecting the one or more target region amplicons. In some embodiments, the one or more target regions are a set of target regions, and in certain embodiments the one or more primer pairs are a set of primer pairs, each being configured for amplifying one target region of the set. In some embodiments, the method further comprises quantifying an amount for at least one of the one or more target region amplicons.

In some embodiments performing one or more amplifications comprises performing a universal amplification, wherein at least a plurality of the MSRE-exposed, uncleaved, adapted cfDNA are amplified to generate amplicons, and wherein the method further comprises selectively enriching one or more target regions from the amplicons using one or more hybrid-capture probes to generate enriched DNA molecules comprising the one or more target regions. In some embodiments, the method further comprises detecting the one or more enriched DNA molecules comprising the one or more target regions. In some embodiments, the one or more target regions are a set of target regions, and the one or more hybrid-capture probes are a set of hybrid-capture probes, each being configured to selectively bind a sequence in one target region of the set. In some embodiments, the set of hybrid-capture probes includes one or more probes that can bind to two or more target regions of the set. In some embodiments, the method further comprises quantifying an amount of enriched DNA molecules having at least one of the one or more target regions.

In another aspect, provided herein is a method for preparing DNA molecules useful for determining a methylation status of selected regions of the DNA molecules, comprising
a) ligating adapters to circulating free DNA (cfDNA) obtained or derived from a first liquid sample, thereby forming a plurality of adapted cfDNA comprising adapted cfDNA having MSRE recognition sites;
b) contacting the plurality of adapted cfDNA with one or more MSREs, thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA;
c) performing a first amplification step wherein at least a plurality of the MSRE-exposed, uncleaved, adapted cfDNA are amplified;
d) performing a second amplification step to amplify a set of target regions from the amplified MSRE-exposed, uncleaved, adapted cfDNA, each target region having one or more MSRE recognition sites, wherein a target region of the set is amplified and thereby generating target region amplicons if the target region is present in one or more of the amplified MSRE-exposed, uncleaved, adapted cfDNA; and
e) quantifying an amount for at least some of the target region amplicons of the set by performing a next-generation sequencing reaction on clonally amplified target region amplicons, or amplicons derived therefrom.

In some embodiments, the first amplification step comprises one or more PCRs using universal primers. In some embodiments, the second amplification step comprises one or more PCRs using a set of primer pairs, wherein each pair of the set is designed to amplify one target region having one or more MSRE recognition sites, and wherein at least one primer of each primer pair is a target-specific primer. In some embodiments, the second amplification step comprises one or more LTC reactions using a set of probe-dependent primer pairs, wherein each pair of the set is designed to amplify one target region having one or more MSRE recognition sites.

In one aspect, provided herein is a method for preparing deoxyribonucleic acids (DNA), comprising a methylation-sensitive restriction enzyme (MSRE) recognition site, comprising
a) ligating adapters to sample DNA obtained or derived from a first sample from a first subject, thereby forming adapted DNA molecules comprising adapted DNA molecules having methylation sensitive restriction enzyme (MSRE) recognition sites;
b) contacting the adapted DNA molecules with one or more MSREs, thereby generating MSRE-exposed, adapted DNA molecules, wherein a plurality of the MSRE-exposed, adapted DNA molecules having an unmethylated MSRE recognition site are cleaved by at least one of the MSREs; and
c) performing one or more targeted polymerase chain reactions (PCRs) using a PCR reaction mixture comprising the MSRE-exposed, adapted DNA molecules and one or more primer pairs designed to amplify one or more target regions in the MSRE-exposed, adapted DNA molecules, each target region comprising one or more MSRE recognition sites, wherein one or more of the one or more target regions are amplified if all of the MSRE recognition sites in the MSRE-exposed, adapted DNA molecules comprising the one or more target regions are methylated.

In another aspect, provided herein is a method for preparing DNA molecules useful for determining a methylation status of selected regions of the DNA molecules, comprising
a) ligating adapters to circulating free DNA (cfDNA) obtained or derived from a first liquid sample, thereby forming a plurality of adapted cfDNA comprising adapted cfDNA having MSRE recognition sites;
b) contacting the plurality of adapted cfDNA with one or more MSREs, thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA;
c) amplifying at least a plurality of the MSRE-exposed, uncleaved, adapted cfDNA;
d) selectively enriching subsets of amplified MSRE-exposed, uncleaved, adapted cfDNA having one or more target regions using a set of hybrid capture probes, wherein each target region comprises one or more MSRE recognition sites, and wherein each probe of the set is designed to hybridize to one target region; and
e) quantifying an amount for at least some of the enriched subsets by performing a next-generation sequencing reaction on clonally amplified enriched subsets, or amplicons derived therefrom.

In some embodiments, the methods disclosed herein further comprise determining the methylation status of one or more target regions. In some embodiments, the determining comprises comparing the amount quantified for the target region amplicons derived from a first sample to a preset value. In some embodiments, the determining comprises comparing the amount quantified for the target region amplicons derived from a first sample to an amount quantified for target region amplicons derived from a second sample using the same method.

In some embodiments, the amplifying comprises one or more PCRs using universal primers. In some embodiments, the one or more target regions are a set of target regions.

While the embodiments of the present disclosure are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1. Comparison of MSRE Digestion Before Versus after Adapter Ligation

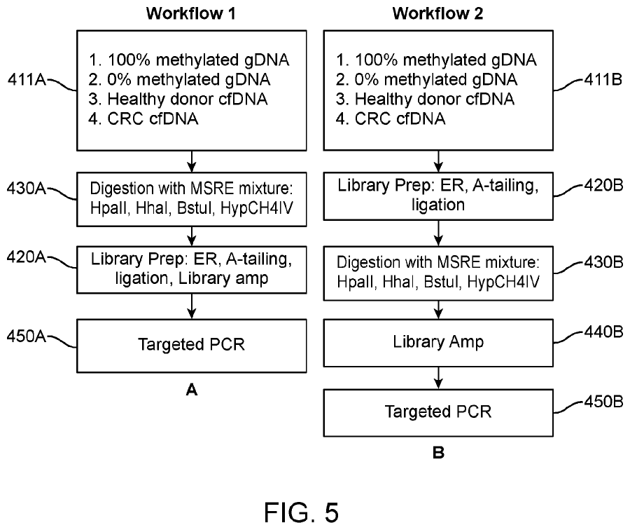

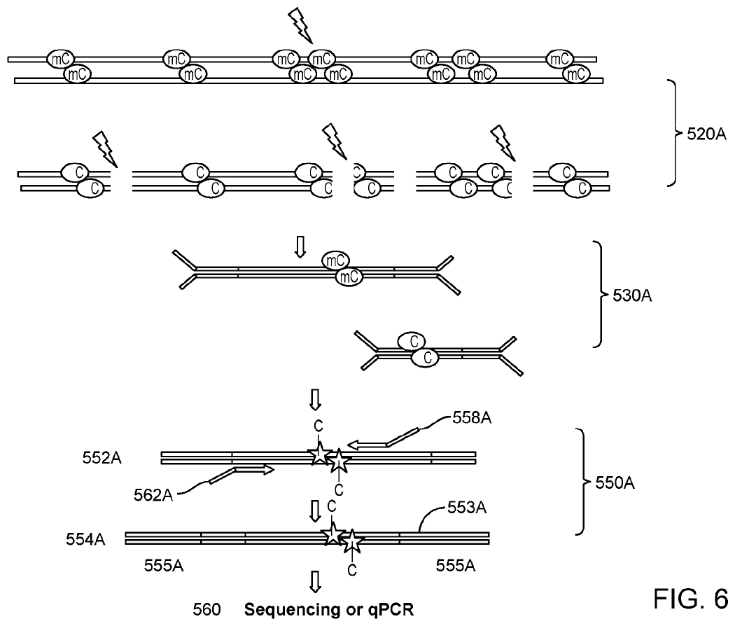

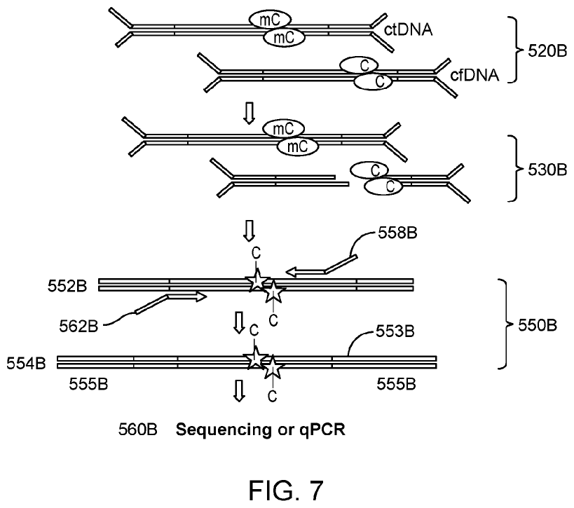

This example demonstrates comparison of two workflows for determining the methylation status of target DNA molecules in cfDNA samples from healthy donors and CRC patients. The workflows differ in the order of the step in which MSREs are used to digest sample DNA molecules in relation to the step of adapter ligation in library preparation, as illustrated in FIG. 5A and FIG. 6 (Workflow 1), and FIG. 5B and FIG. 7 (Workflow 2), respectively.

It was hypothesized that Workflow 1 (MSRE digestion before ligation of adapters to DNA molecules from a sample) would avoid losing information/reads through unexpected digestion from MSRE sites outside prospective amplicons. However, MSRE digestion before ligation of adapters would likely result in increased background noise and inefficient preamplification, which would be biased towards generating amplicons from shorter, digested fragments typically not having methylated targets. On the other hand, given that CpG sites in certain regions of cancer cells are known to be predominantly co-methylated, the potential for information/read loss with Workflow 2 (MSRE digestion after adapter ligation) can be minimized or even negligible with proper target profiling. Moreover, MSRE digestion after adapter ligation would likely increase assay specificity and allow more flexibility in target-specific primer placement/design, as adapted DNA molecules comprising unmethylated off-amplicon MSRE sites would be cleaved by the MSREs and would not be amplified in the universal amplification step.

As illustrated in FIG. 5A, the first workflow (Workflow 1) comprises digesting DNA samples with a mixture of MSREs before any library preparation. As illustrated in FIG. 5B, the second workflow (Workflow 2) comprises digesting DNA samples with a mixture of MSREs after adapter ligation. The DNA reactants and DNA products of the workflows of FIG. 5A and FIG. 5B are shown in FIG. 6 and FIG. 7, respectively. For both workflows, an MSRE cocktail/mixture of HpaII, HhaI, BstU1, and HpyCH4IV, or a control solution lacking any MSREs, were used in the digestion step in the experiments described herein. The MSREs were selected based upon MSRE recognition sites in the prospective target region amplicons.

Unless otherwise noted, DNA samples comprising (i) 100% methylated contrived human gDNA, or (ii) 100% unmethylated contrived human gDNA, or (iii) cfDNA from a healthy subject, or (iv) cfDNA from a CRC patient were used for the experiments described herein (411A, 411B). Samples were spiked with 0.01% unmethylated lambda phage and 0.0006% methylated pUC19 DNA by mass as a control and to normalize results in some experiments. Human gDNA, as well as lamda+pUC19 DNA, samples were sheared using an R230 sonicator and an AFA-TUBE TPX plate (Covaris) to mimic the size of cfDNA before use and size selected before use.

In Workflow 1, the DNA samples (411A) were then treated with an MSRE mixture or a control solution lacking any MSREs (430A, 520A). 30 ng of DNA in a 40 μl volume was added to the MSRE cocktail comprising 0.125 μl of each of the following MSREs (NEB): 20 U/μl HpaII, 20 U/μl Hhal, 10 U/μl HpyCH4IV, and 10 U/μl BstU1 in 4.5 μl of 10× CutSmart buffer (New England Biolabs), or to the control solution containing 4.5 μl of 10× CutSmart Buffer (NEB) in 0.5 μl water only. Digestion was performed at 37° C. for 1 hr, 60° C. for 30 min, and inactivation at 80° C. for 20 min. Samples were cleaned with 3× Ampure XP beads (Beckman Coulter) to retain the potential small fragments and eluted with 40 μl DSB-Tween 20 elution buffer.

In Workflow 1, the treated DNA samples were then blunt-ended, A-tailed, and ligated with an adapter without MSRE sites or is fully methylated, and the ligated DNA samples were universally amplified (420A, 530A). Specifically, the DNA samples were blunt-end repaired using Klenow Large Fragment Polymerase and T4 polynucleotide kinase (PNK) (Thermofisher), and A-tailed using Klenow Fragment exonuclease (3′-5′ exo) (Thermofisher). Nucleic acid adapters lacking internal MSRE sites or fully methylated were ligated using T4 ligase (Thermofisher) and 5′ ends were phosphorylated using PNK resulting in Y adapted fragments (530A, FIG. 6). The MSRE treated, adapted DNA molecules were purified using Kingfisher AMPure XP-Purification protocol, and eluted with a Tris buffer solution. Library amplification was performed by performing PCR using universal primers designed to bind to primer binding sites on adapters on the Y adapted DNA molecules, and Kapa HiFi Polymerase (Kapa Biosystems) on a Veriti Thermocycler set for 20 cycles (Applied Biosystems) resulting in amplified, adapted DNA molecules (552A of FIG. 6).

For Workflow 2, the protocols for individual steps were the same as Workflow 1, but DNA samples (4111B) were blunt-end repaired, A-tailed, and the adapter was ligated as described to produce Y adapted DNA samples (420B, 520B) prior to the MSRE digestion step (430B, 530B). The MSRE-treated, adapted DNA samples were then universally amplified (440B) as described above resulting in amplified, adapted DNA molecules (552B of FIG. 7).

In both workflows, following universal amplification, targeted amplifications were performed using target-specific primer pairs (450A, 450B, 550A, 550B). Specifically, targeted PCR reactions were performed to amplify target regions within the universally amplified adapted DNA molecules using about 220 primer pairs designed to amplify regions understood to be hypermethylated in CRC patients. To avoid primer-primer interactions while maximizing primer selection, primers that interact with one another were split into two pools, pool 1 and pool 2.

PCR reaction mixes were prepared using pooled target-specific primer pairs (558A/B, 562A/B). PCR reactions were run on a Veriti PCR machine with an initial denaturation step at 98° C. for 2 minutes, and 14 cycles of the following steps: 95° C. for 30 seconds, 62.5° C. for 15 minutes, 72° C. for 30 seconds or alternatively, 1 minute, followed by a final extension of 72° C. for 2 minutes, resulting in amplified DNA molecules (554A/B) that include a target region or a portion thereof.

6 μl of each well of the targeted PCR reaction wells were pooled, and frozen. For sequencing reactions, pooled samples were purified using QiAquick PCR purification column (Qiagen), with a final elution of 50 μl of elution buffer. Purified pooled samples were quantified using qPCR and also run on an Agilent BioAnalyzer DNA High-Sens assay at 1:20 dilution in triplicate.

Sequencing (560A/B) was performed at 2×50 bp. A total of 56 samples were analyzed, with human targets depth of read greater than 100K. Data was analyzed focusing on enrichment of methylated target DNA molecules vs. depletion of unmethylation target DNA molecules in the genomic DNA samples. Furthermore, performance of the two workflows was compared for cfDNA from healthy donors vs. CRC patients. Other key targets were evaluated including on target percent, uniformity, and drop-out rate.

### Enzyme Efficiency Assessment Using Lambda Control

Efficiency of the MSRE enzymes was first calculated using Workflow 1 (W1) based on digestion of unmethylated lambda DNA. It was found that BstU1, HhaI, and HpyCH4IV each had an efficiency of about 99.5%, while HpaII had an efficiency of about 75% (data not shown).

Efficiency of the MSRE enzymes was further assessed using both Workflow 1 and Workflow 2 based on digestion of unmethylated lambda DNA. The unmethylated lambda DNA samples were treated either with the MSRE cocktail or a control solution without any MSRE. Data for lambda targets containing one or more MSRE recognition sites were extracted and analyzed separately for each MSRE in the cocktail. For each enzyme, depth of read (DOR) of amplicons comprising one or more recognition sites for the enzyme, normalized to the median DOR of methylated pUC19 control, were analyzed and compared between treated (MSRE) and control (Control) samples, as well as Workflow 1 (W1) and Workflow 2 (W2).

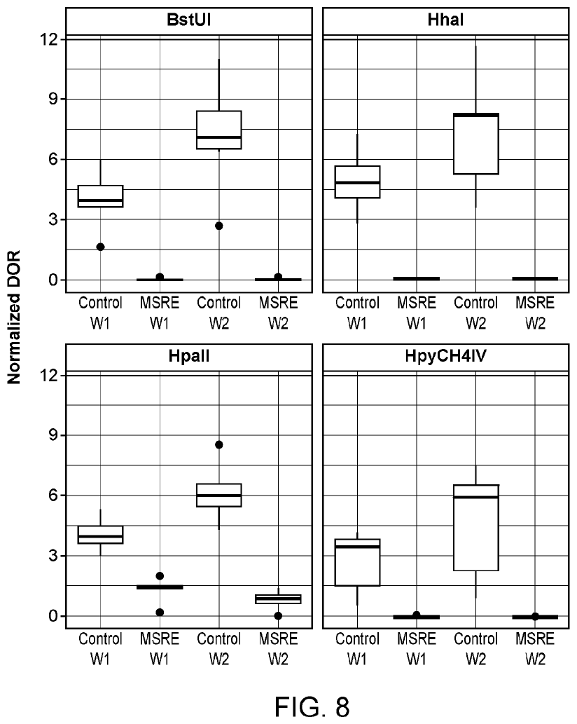

As seen in FIG. 8, the samples treated without any MSRE (Control.W1 and Control.W2) had a normalized DOR at about 4 for W1 and about 6 for W2. For the samples treated with the MSRE cocktail (MSRE.W1 and MSRE.W2), the results were similar for BstUI, HhaI, and HpyCH4IV, with a normalized DOR near zero for both workflows, while for HpaII, measurement of normalized DOR was at about 1.5 for W1, and about 1 for W2, thus corresponding to the respective restriction enzymes' efficiencies determined in prior experiments. Overall, these results demonstrate that unmethylated lambda controls are sensitive to MSRE digestion, and that both Workflow 1 and Workflow 2 perform in a similar manner on a control unmethylated lambda sample.

Enzyme Efficiency and Workflow Specificity Assessments Using Contrived 100% and 0% Methylated Human gDNA Samples

Next, efficiency of the MSRE enzymes under Workflow 1 and Workflow 2 was determined using contrived human gDNA. Contrived 100% methylated or 0% methylated human gDNA samples were treated either with the MSRE cocktail or a control solution without any MSRE using Workflow 1 or Workflow 2. Depth of read (DOR) was calculated for the following samples using Workflow 1 and Workflow 2: 100% methylated gDNA samples treated with the mixture of MSREs, 100% methylated gDNA samples treated with a control mixture (no MSRE), 0% methylated gDNA samples treated with the MSRE mixture, and 0% methylated gDNA samples treated with the control mixture. Data for MSRE targets containing one or more cut sites were extracted and analyzed separately for each MSRE in the cocktail. For each enzyme, depth of read (DOR) of amplicons comprising one or more recognition sites for the enzyme, normalized to the median DOR of methylated pUC19 control, were analyzed and compared between Workflow 1 (W1) and Workflow 2 (W2), as well as treated (MSRE) and control (Control) samples.

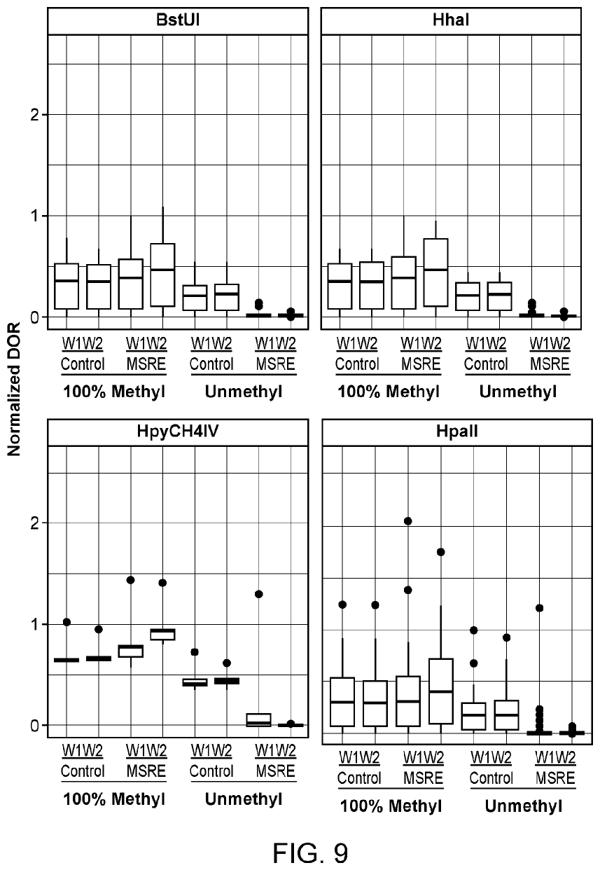

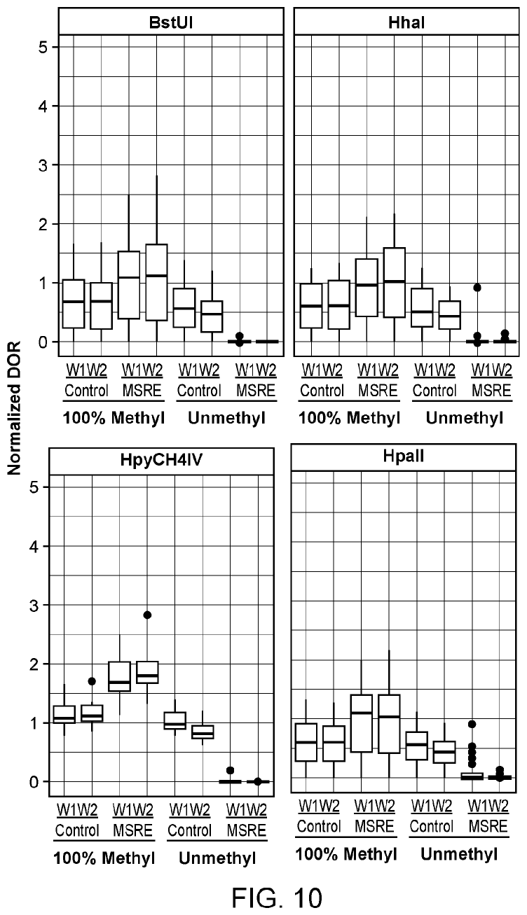

Results are shown for each of the targeted primer pools. FIG. 9 (Pool 1) and FIG. 10 (Pool 2) show comparisons of normalized DOR readout of two pools of human targets for each MSRE recognition site between 100% methylated and 0% methylated human gDNA samples either treated with control or MSRE mixture using W1 or W2. As expected, HpaII demonstrated less efficiency, primarily with the use of the targeted primers in pool 1 (FIG. 9, HpaII results). However, overall it was found that for both targeted primer pools, MSRE digestion before the library prep (W1, FIG. 5A) and after ligation (W2, FIG. 5B) showed sufficient cutting in unmethylated samples (FIG. 9, last 2 columns of each graph, and FIG. 10, last two columns of each graph; labeled as Unmethyl W1-MSRE and Unmethyl W2-MSRE), but preserved (undigested) in 100% methylated samples (FIG. 9, columns 3-4 of each graph, and FIG. 10, columns 3-4 of each graph; labeled 100% Methyl W1-MSRE & 100% Methyl W2-MSRE). Thus, these results demonstrate that unmethylated contrived human gDNA was sensitive to MSRE treatment, and that both workflows were sufficient at differentiating methylated vs. unmethylated human gDNA.

DOR Comparison Between cfDNA Samples from CRC Patients and Healthy Donors Using Workflow 1 and Workflow 2

To evaluate whether signals from healthy vs. CRC patients could be distinguished using Workflow 1 and Workflow 2, individual performance in human amplicons for cfDNA samples from healthy donors and CRC patients was assessed using pooled target-specific primer pairs designed to amplify regions understood to be hypermethylated in CRC patients.

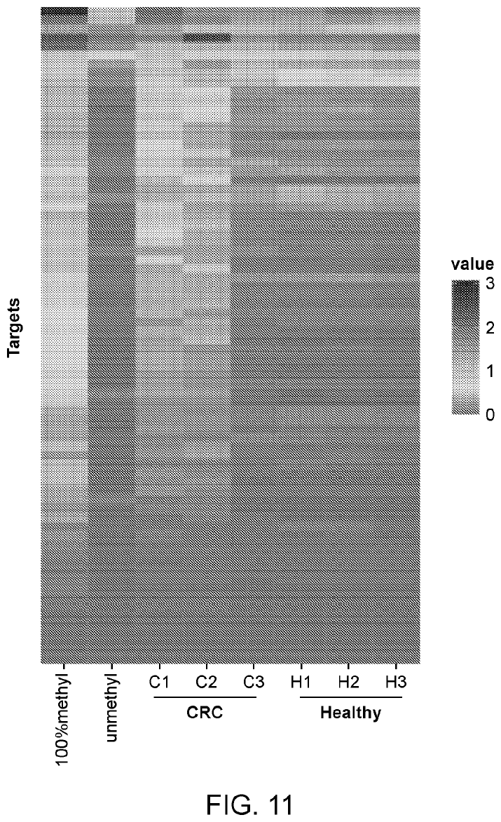

First, cDNA samples from CRC patients vs. healthy donors were analyzed using Workflow 1 protocol. All assays were normalized using non-MSRE sites. FIG. 11 shows a heat map of normalized DOR of pool 1 targets from samples of three individual CRC patients C1, C2 and C3 (columns 3, 4, 5, respectively) compared to 3 healthy individuals H1, H2, and H3 (columns 6, 7, and 8). The VAFs for C1, C2, and C3 samples were at 25.31%, 20.52%, and 3.18%, respectively. 100% methylated human gDNA and unmethylated human gDNA controls are shown in columns 1 and 2, respectively. Using primer pool 1 and Workflow 1 protocol, high VAF samples C1 (25.31%) and C2 (20.52%) showed clear DOR enrichment compared to healthy samples with primer pool 1 targets, whereas lower VAF sample C3 had similar DOR compared to healthy samples. The background of healthy donors was not as clean as unmethylated gDNA.

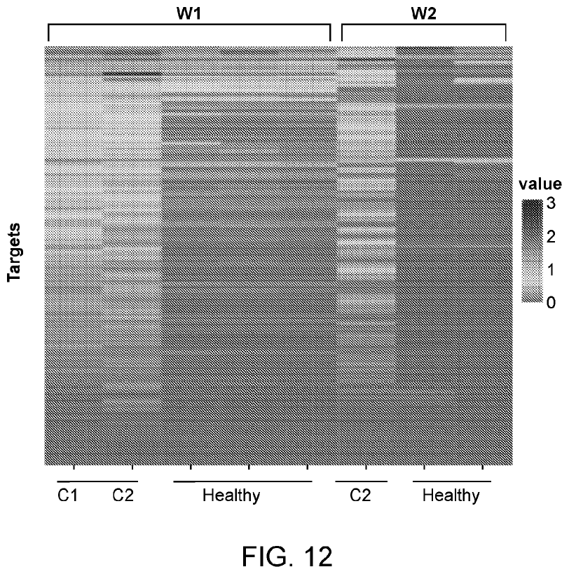

Next, high VAF cfDNA samples from CRC patients vs. healthy samples were analyzed using either Workflow 1 (W1) or Workflow 2 (W2) protocols, and the results from the two pools were combined. All assays were normalized to pUC19 median. FIG. 12 shows a heat map of normalized combined DOR of pool 1 and pool 2 targets from samples of two individual CRC patients (C1 and C2) compared to three or two healthy individuals using Workflow 1 (W1) or Workflow 2 (W2). The VAFs for C1 and C2 samples were at 25.31% and 20.52%, respectively. Both W1 and W2 showed good differentiation between high VAF samples C1 (25.31%) and C2 (20.52%) vs. health cfDNA samples. W2, however, showed a much cleaner background in healthy cfDNA samples compared to W1.

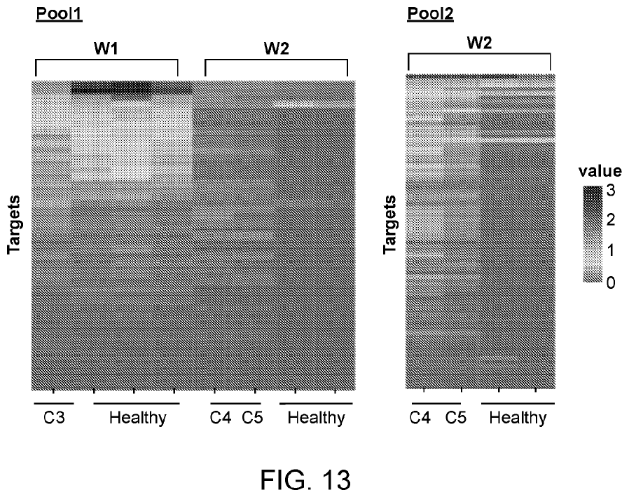

Next, CRC patient cfDNA samples with lower VAFs vs. healthy cfDNA samples were analyzed using either Workflow 1 (W1) or Workflow 2 (W2) protocols. All assays were normalized to pUC19 median. In FIG. 13, the left panel shows a heat map of normalized DOR of pool 1 targets from samples of three individual CRC patients C3, C4 and C5 compared to healthy individuals using either W1 or W2 protocols. The right panel shows a heat map of normalized DOR of pool 2 targets from C4 and C5 samples compared to healthy individuals using W2 protocol. The VAFs for C3, C4, and C5 samples were at 3.19% 2.51%, and 1.78%, respectively. As can be seen from FIG. 13, W2 has much cleaner background in healthy cfDNA and better resolution in lower VAF CRC samples compared to W1. This is an important advantage for Workflow 2 since cfDNA samples for cancer screening or diagnostic assays, including for early cancer screening assays, typically have a low ctDNA VAF. The mean VAF of stage II and III CRC samples, for example, are <0.1%.

To test the limit of detection with Workflow 2 further, the analytical resolution of Workflow 2 with 1%, 0.5%, 0.1%, 0.05%, and 0% (by mass) of contrived fully methylated human gDNA was measured using pool 1 targets and Workflow 2 protocol as discussed above. DORs were normalized to the median DOR of human non-MSRE control. Samples with 1%, 0.5%, 0.1%, or 0.05% fully methylated gDNA were able to be differentiated from samples with 0% fully methylated gDNA.

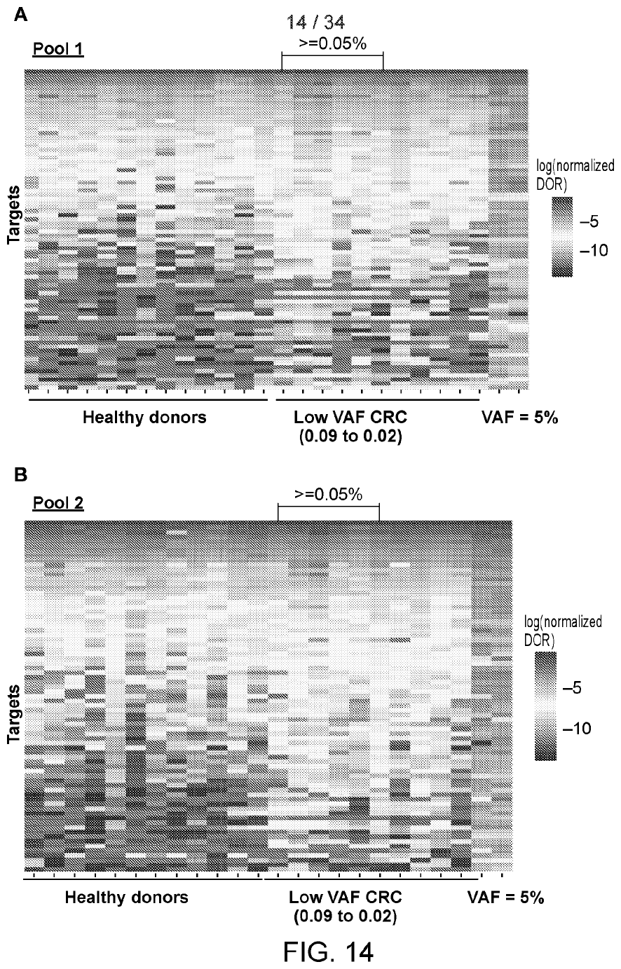

Additional cfDNA samples were also tested from 13 healthy subjects and 13 CRC patients (11 VAF<0.1% and 2 VAF=5%) with Workflow 2 protocol as discussed above. All sample DOR were normalized to the median of human non-MSRE sites. As can be seen in FIG. 14A (pool 1) and FIG. 14B (pool 2), most CRC cfDNA samples with VAF>0.05% showed different patterns compared to cfDNA samples from healthy donors. The targets in primer pool 2 had better resolution in low VAF samples compared to primer pool 1, especially in VAF between 0.09% to 0.05%, suggesting that analytical resolution of Workflow 2 can be further improved by target profiling and selection and/or further optimization of the protocol.

### Example 2. Clinical Performance of Workflow 2

To evaluate the clinical performance of the MSRE approach and demonstrate its potential use in early cancer detection and recurrence monitoring, a methylation-based classifier with machine learning algorithms was developed and used to distinguish between patients with colorectal cancer (CRC), patients in remission, and healthy individuals. First, >800 CRC-specific CpG targets were identified for initial evaluation by comparing the methylation landscape of CRC and normal samples (tissue and blood) available in The Cancer Genome Atlas (TCGA) database and Gene Expression Omnibus (GEO) datasets. To develop and test the classification model, 50 ctDNA-positive CRC patients (24% stage I, 40% stage II, 24% stage III, and 12% stage IV), 10 ctDNA-negative patients (CRC patients in remission), and 36 healthy normals were included in the analysis.

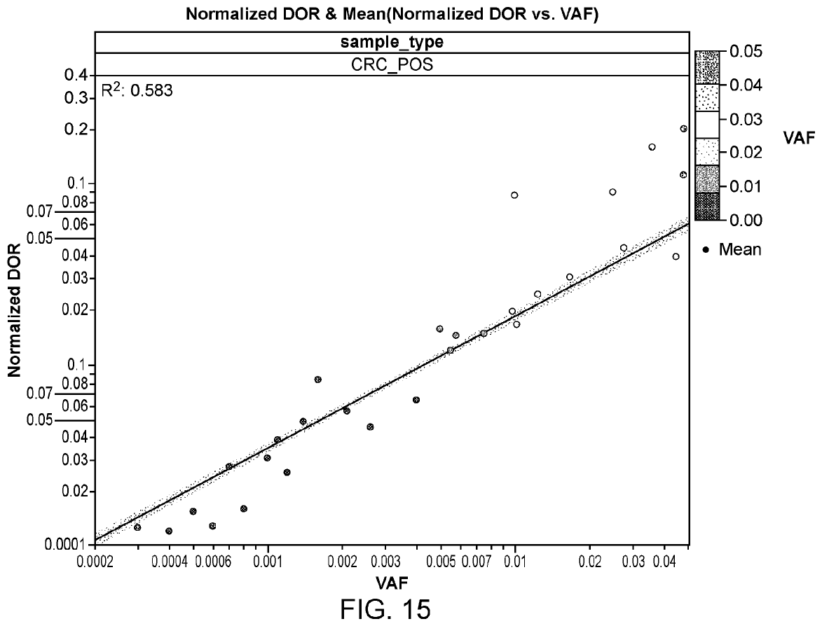

Workflow 2 was utilized, as described above (see, FIG. 5B and FIG. 7). A machine learning model was used to evaluate the highest performing CpG targets among the >800 potential targets that effectively discriminated between CRC ctDNA-positive patients and healthy individuals. The median variant allele frequency (VAF) of single nucleotide variants (SNVs) of the CRC ct-DNA positive samples was ~0.1%, with the majority of samples ranging from 0.01-1%, and few samples with 1-5% VAF. Using the highest performing CpG targets, the observed methylation level was correlated with the SNV VAF detected by ctDNA testing (R2: 0.8). For example, FIG. 15 demonstrates that higher DOR is associated with higher SNV VAF using the top 40 targets.

CRC Vs. Normal Differentiation

To evaluate whether the MSRE Workflow 2 approach can differentiate CRC ctDNA positive samples from healthy or ctDNA negative samples, MSRE-treated libraries were prepared as described above from 50 CRC ctDNA positive, 10 CRC ctDNA negative, and 36 healthy donor samples. 196 CRC-specific CpG regions were assayed in two pools (pool 1 and pool 2) with mPCR using pooled target-specific primer sets (98 sets each pool) and sequenced. Similar on-target rate was observed across CRC positive, CRC negative, and healthy samples.

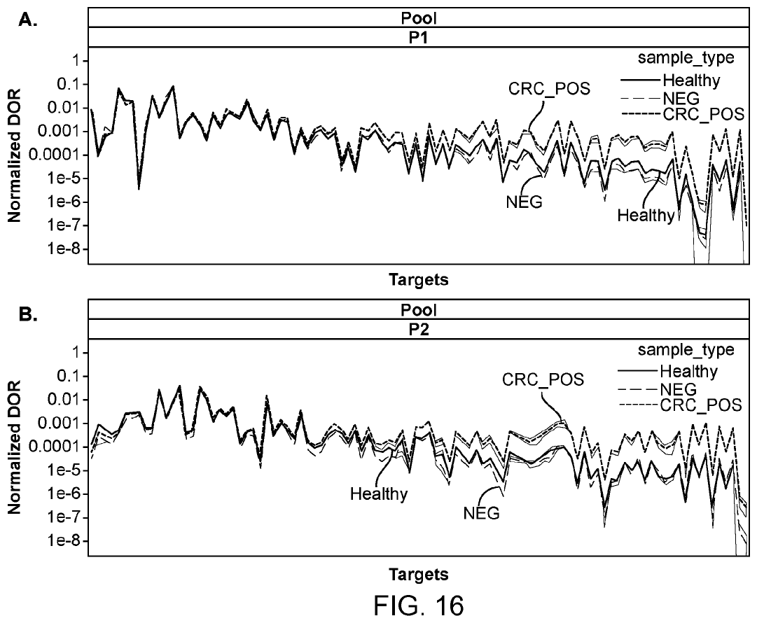

Assay DORs were normalized using total reads and then compared for different sample groups, as shown in FIG. 16A (pool 1) and FIG. 16B (pool 2). Solid line is the mean of normalized DOR, and shading is +/−standard error. The targets were sorted by mean DOR ratio in CRC/healthy samples in the x-axis. A subset of targets showed higher CRC/healthy DOR ratio and 53/196 assays provided >10 of CRC/healthy DOR ratio.

CRC Vs. Healthy Classification Using Random Forest Method

To generate a model for classifying healthy vs CRC samples, samples were split into 60% training set (57 samples) and 40% test set (39 samples). A random forest model was generated using the training set to differentiate CRC positives from healthy samples.

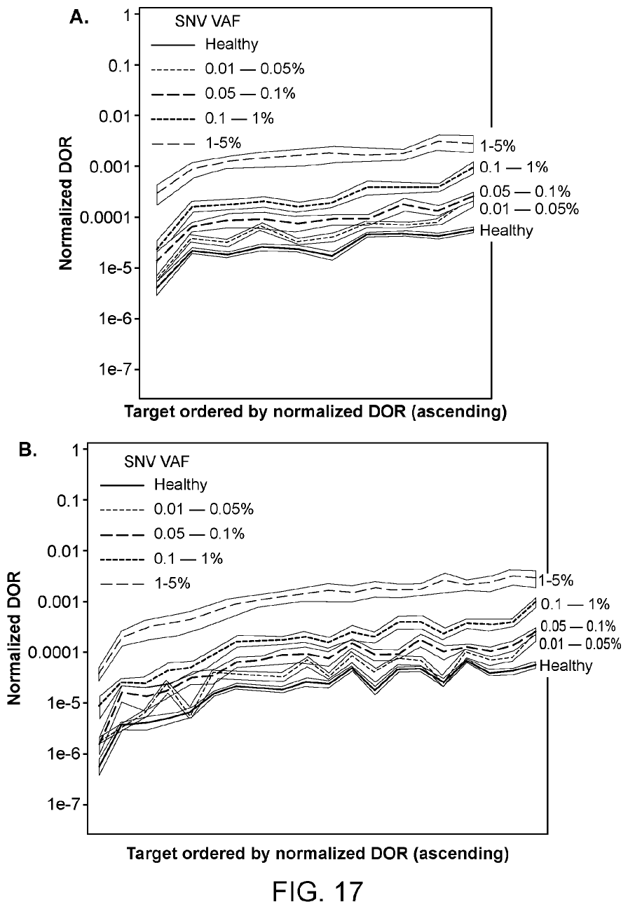

The methylation targets were ranked by Random Forest feature Gini importance, which calculates each feature importance as the sum over the number of splits. When using the top targets, CRC samples with >0.05% SNV VAF demonstrated better separation from healthy samples. The majority of top target assays can provide differentiation between 0.01-0.05% VAF CRC and healthy samples (FIG. 17A and FIG. 17B).

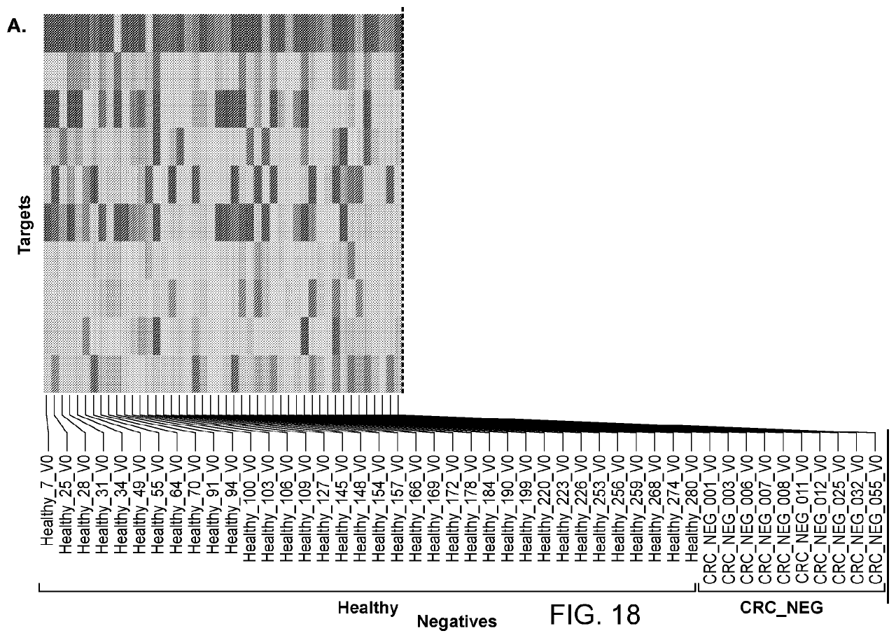

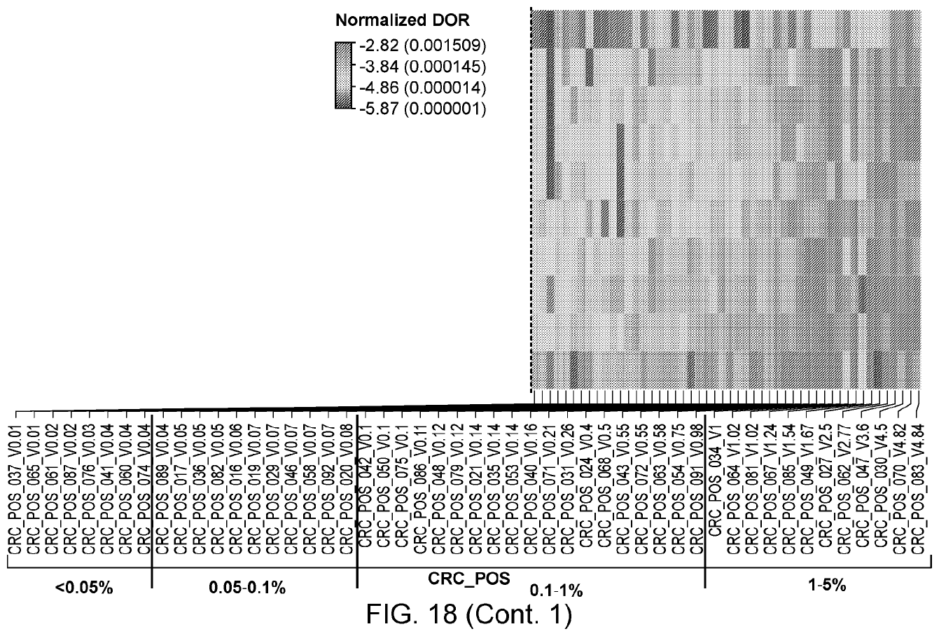

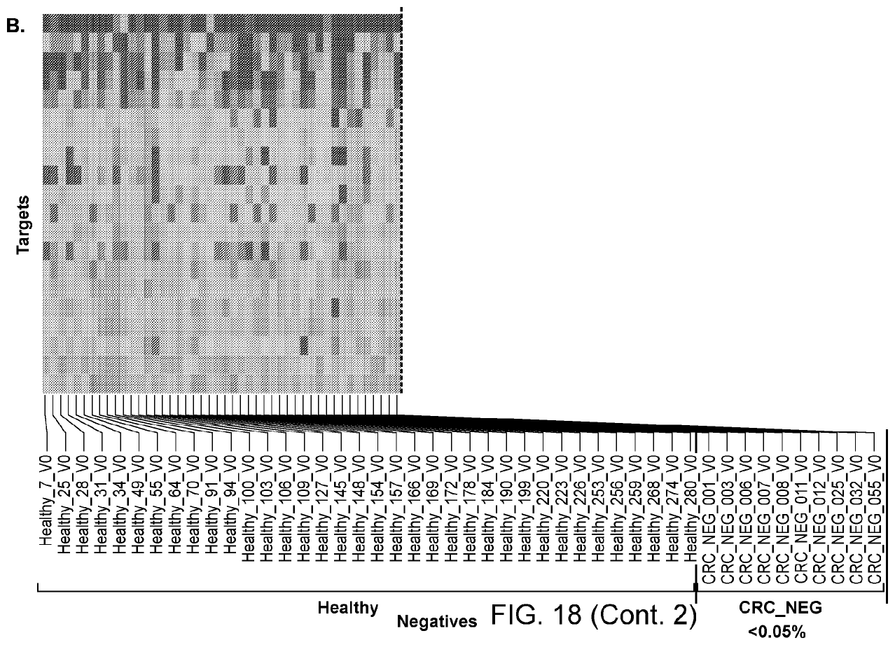

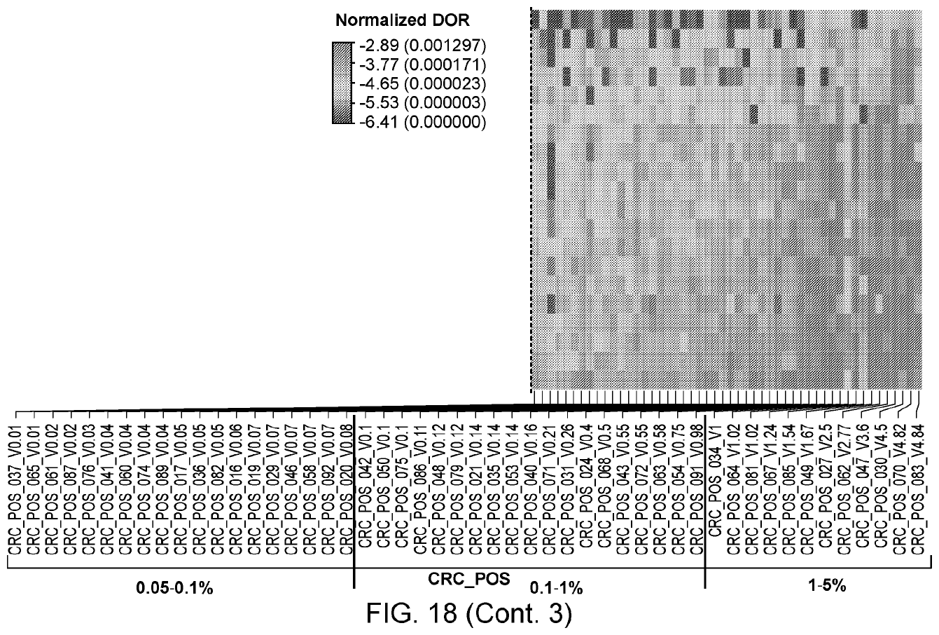

As shown in the heat maps (FIG. 18A and FIG. 18B), across the top targets, samples with >0.05% VAF can be differentiated from negative samples. Some sample-to-sample variations were observed within each sample group, which may be due to methylation signal and SNV signal not perfectly correlating.

### MSRE W2 Performance

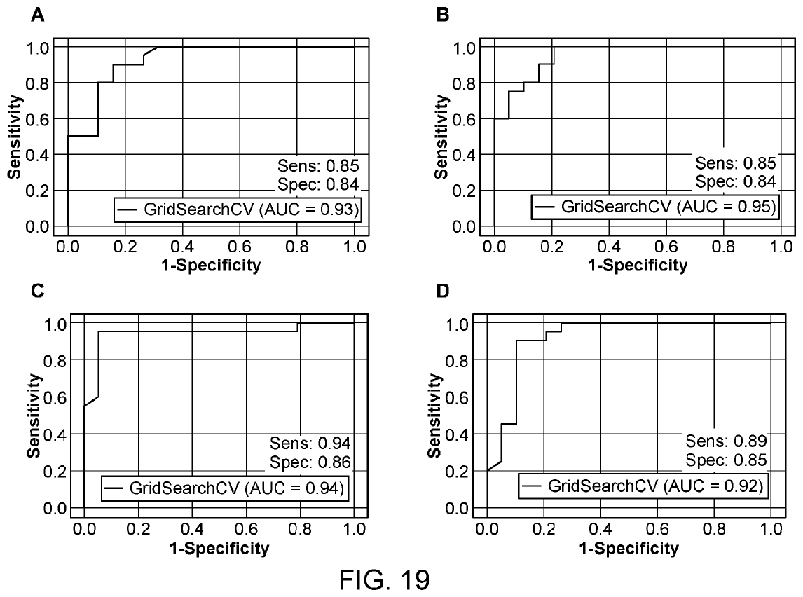

To evaluate the performance of the MSRE Workflow 2 using the Random Forest model approach, the training set was used to generate Random Forest models using either all targets (FIG. 19A) or a subset of targets (FIGS. 19B-D). As shown in Table 1 and FIG. 19C, the top 20 targets in pool 1 provide the best sensitivity and specificity among the generated models, in which specificity of 86%, Sensitivity of 94% was observed from the test set of 39 samples. AUC of 0.92-0.95 was observed across all generated models when classifying healthy vs. CRC samples. This is the probabilistic interpretation of the AUC.

At 90% specificity, up to ~95% sensitivity was observed using top 20 targets in pool 1 (FIG. 19C).

TABLE 1
Comparison of target pools

| | | Top 20 high DOR | Top 20 high | Top 20 high |
|---|---|---|---|---|
| | | ratio targets | DOR ratio | DOR ratio |
| | All Targets | in P1/P2 | targets in P1 | targets in P2 |
| | (FIG. 19A) | (FIG. 19B) | (FIG. 19C) | (FIG. 19D) |
| Sensitivity | 85% | 85% | 94% | 89% |
| Specificity | 84% | 84% | 86% | 85% |
| AUC | 0.93 | 0.95 | 0.94 | 0.92 |

### MSRE Target Assay Investigation

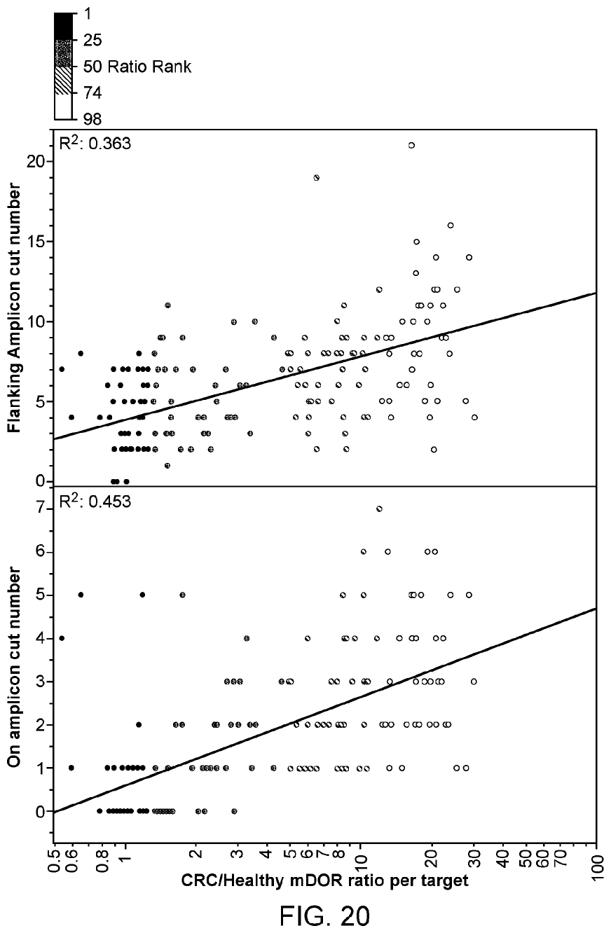

To understand the features of target assays that can provide a better differentiation of CRC vs healthy, the correlation of number of cut sites with the ratio of CRC/healthy mean DOR was examined. It was determined that assays with more MSRE cut sites provide greater differentiation (higher CRC/healthy DOR ratio, FIG. 20).

### Example 3. Hybrid Capture of MSRE-Treated Libraries (MSRE+HC)

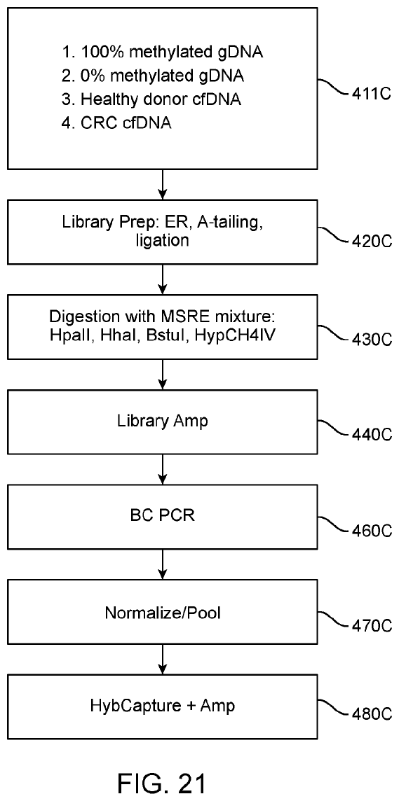

Compared to the targeted amplification approach, enrichment by targeted capture may allow evaluation of additional information beyond methylation levels observed with DOR, including fragment-level information that may enhance MSRE-based cancer detection. This example utilized the 96 samples that underwent library preparation and MSRE treatment as described in Example 2 above, followed by enrichment using a hybrid capture panel. As shown in the schematic of FIG. 21, after undergoing amplification of the MSRE-treated library (440C), sample barcode and P5/P7 index sequences were added through barcoding PCR (460C). Briefly, 200 ng of MSRE-treated amplified libraries were barcoded using barcoding primers to add P5/P7 index sequences. PCR reactions were run with an initial denaturation step at 98° C. for 3 minutes, and 5 cycles of the following steps: 98° C. for 20 seconds, 55° C. for 20 minutes, 68° C. for 1 minute, followed by a final extension of 68° C. for 5 minutes. The barcoded libraries were normalized and pooled (470C) prior to hybrid capture using probes specific for a target panel of up to 1482 probes (480C), with up to 12 samples being pooled per hybrid capture reaction. Following the hybridization reaction, post-capture PCR was performed using P5/P7 primers. PCR reactions were run with an initial denaturation step at 98° C. for 45 seconds, and about 14 cycles (depending on the panel size and number of pooled samples) of the following steps: 98° C. for 15 seconds, 60° C. for 30 seconds, 72° C. for 30 seconds, followed by a final extension of 72° C. for 1 minute. The post-capture libraries were then purified and sequenced using paired-end 2×150 bp sequencing on Novaseq SP flowcell with dual index.

### Target Coverage Between CRC Positive and Normal Samples

To evaluate whether signals from normal (healthy or CRC ctDNA negative) vs. CRC ctDNA positive patients could be distinguished using the MSRE+HC workflow, libraries generated from MSRE treated normal and CRC positive samples were captured using an MSRE HC panel as described above. The HC panel was designed to capture 860 CRC-specific CpG regions understood to be hypermethylated in CRC patients, which were identified by comparing the methylation landscape of CRC with adjacent normal tissue or healthy tissue/blood samples available in The Cancer Genome Atlas (TCGA) database and public datasets on the Gene Expression Omnibus (GEO) repository. The panel also included 17 human non-MSRE targets. 855/860 target regions contained MSRE recognition sites, and the majority of the target regions (+/−160 bp of CpG) contained multiple MSRE recognition sites. 261/855 target regions contained recognition sites for all four MSRE, BstUI/HhaI/HpaII/HpyCH4IV, and each region having between 4-38 cut sites with median of 13 cuts. Most target regions (481/855) contained recognition sites for three MSREs, BstUI/HhaI/HpaII, and each region having between 3-42 cut sites with median of 13 cuts.

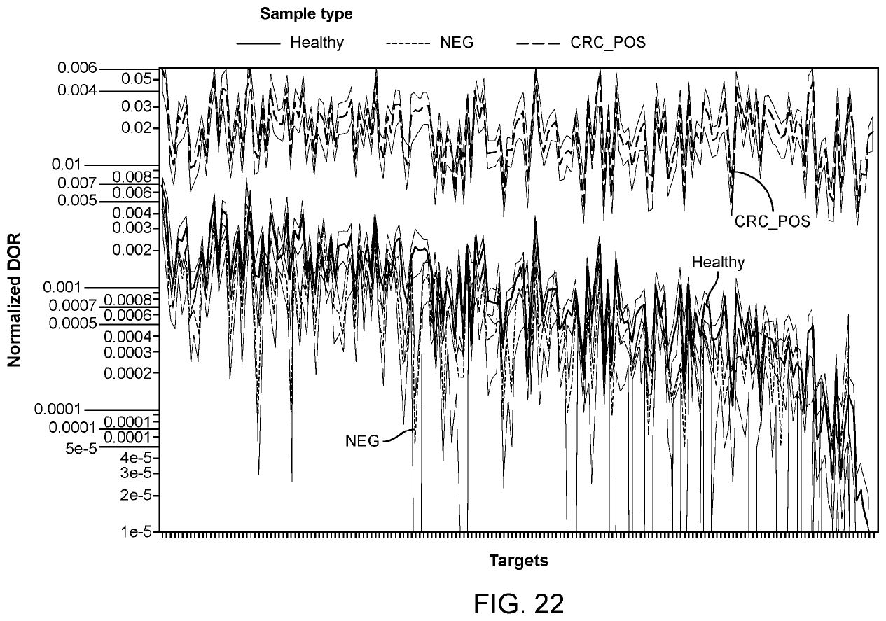

The coverage at CpG targets was calculated after deduplication. The target assay DOR was normalized by non-MSRE control DOR and then compared for the different sample groups. Normalized coverage at the CpG targets for CRC ctDNA positive (CRC_POS), CRC ctDNA negative (NEG), and healthy samples are shown in FIG. 22. The target assays were sorted by mean DOR ratio in CRC/healthy samples in the x-axis. A subset of targets showed higher CRC/healthy DOR ratio and 178/855 assays provided >10 of CRC/healthy DOR ratio.

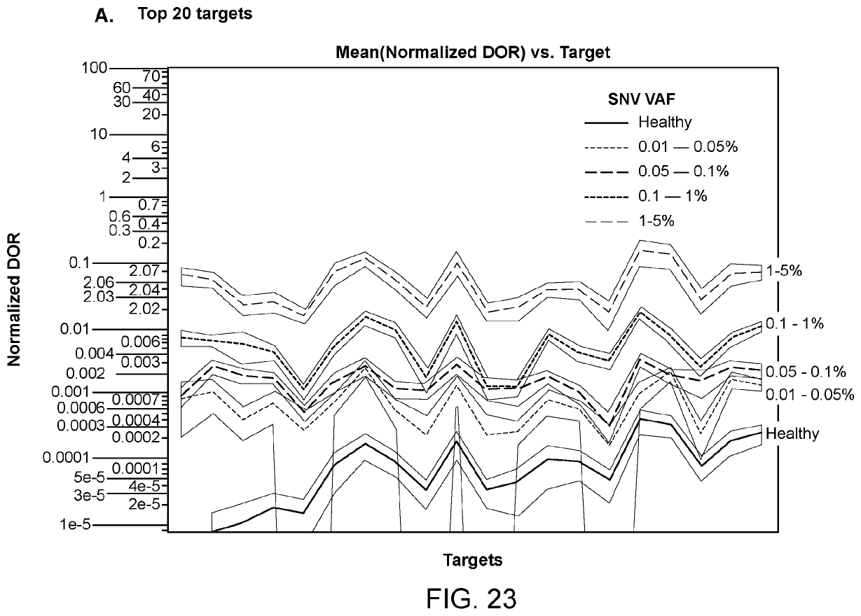

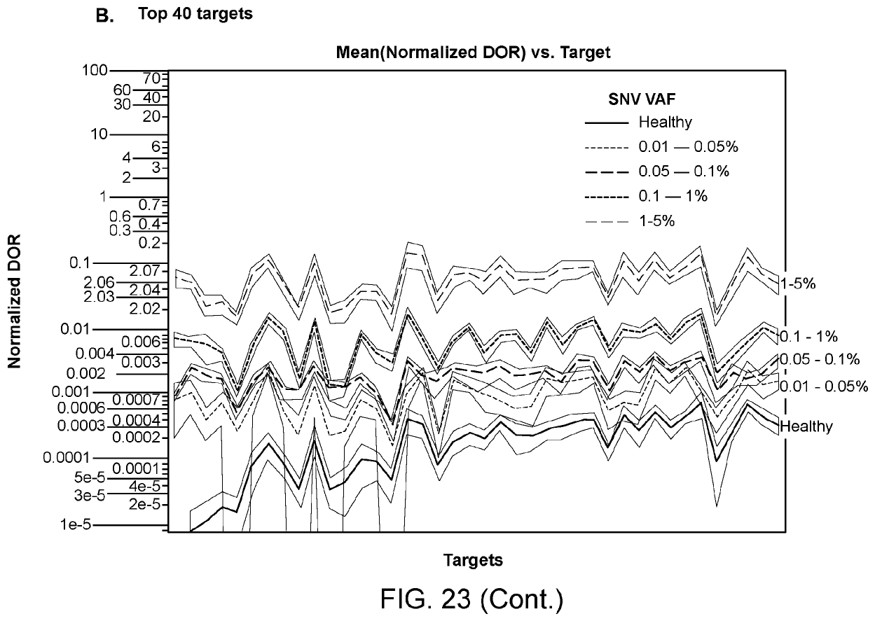

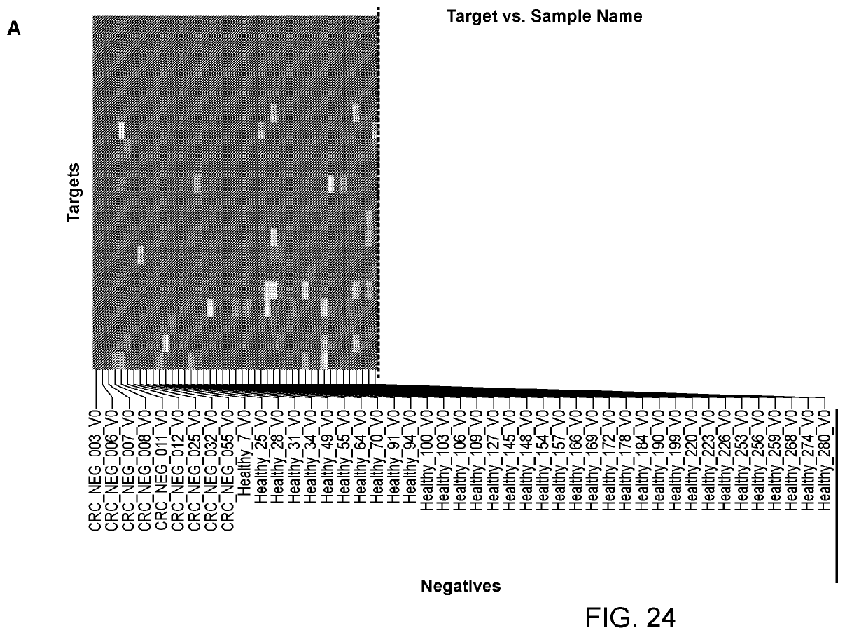

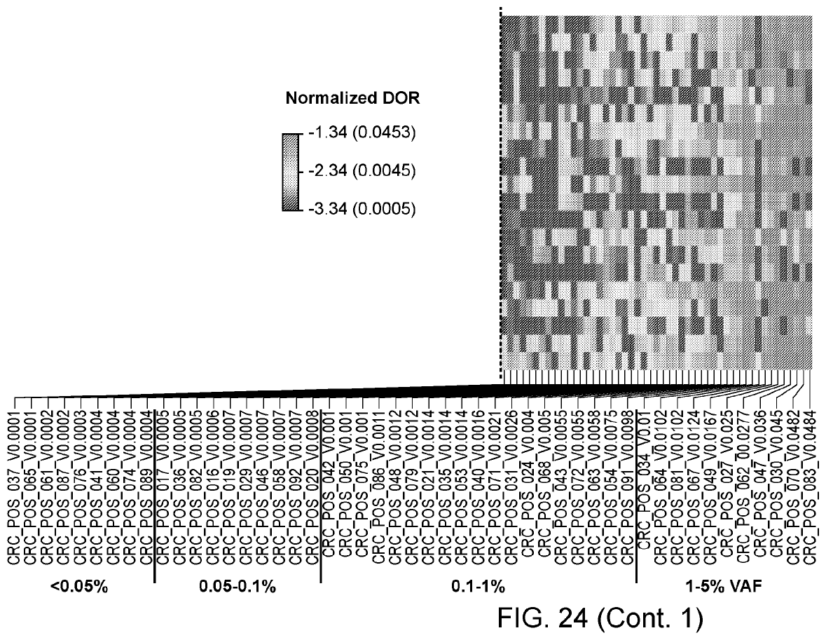

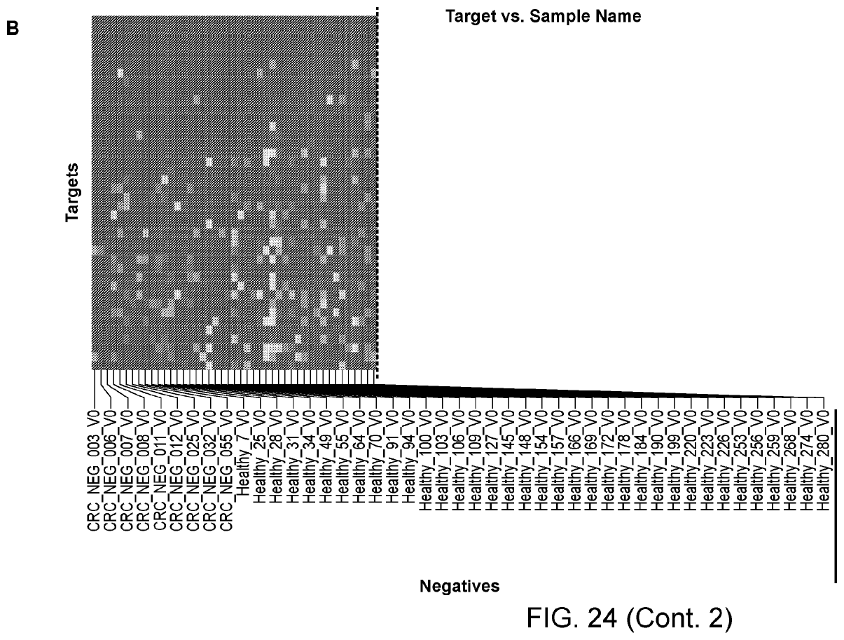

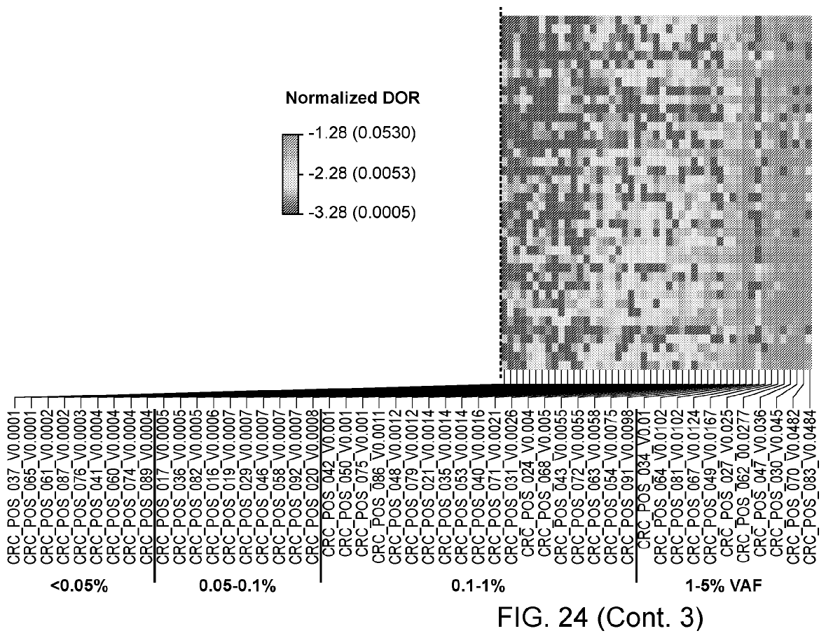

When using top 20 and top 40 targets (ranked by CRC/healthy DOR ratio), the majority of the top target assays can provide differentiation between CRC (0.01%-5% VAF) and healthy samples (FIG. 23). As shown in the heat maps, across the top 20 targets (FIG. 24A) and top 40 targets (FIG. 24B), CRC positive samples (including low VAF samples) can be differentiated from CRC negative and healthy samples. There are some sample-to-sample variations within each sample group.

### MSRE+HC Performance

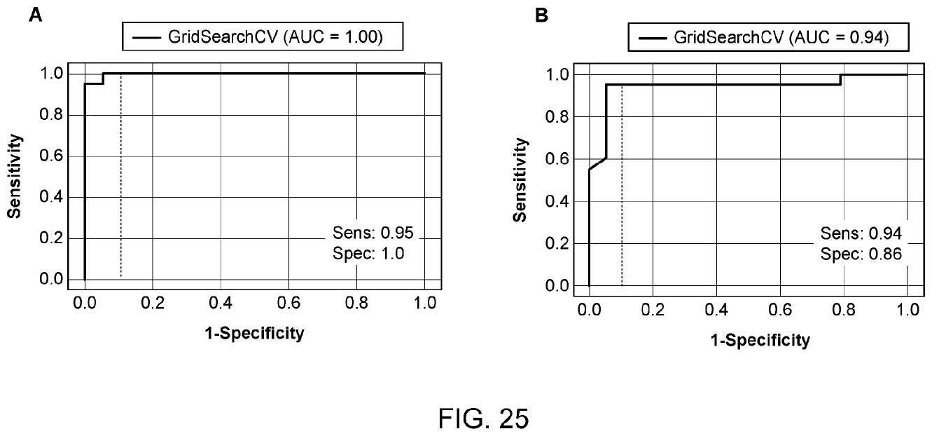

To evaluate the performance of the MSRE+HC workflow using the Random Forest model approach, samples were split into 60% training set and 40% test set, and the training set was used to generate Random Forest models using top 40 high DOR ratio targets. As shown in Table 2 and FIG. 25A, 95% sensitivity and 100% specificity using MSRE+HC workflow was observed from the test set of 39 samples. AUC of 0.99 was observed when classifying healthy vs. CRC samples. This is the probabilistic interpretation of the AUC.

Both MSRE+HC workflow and MSRE+mPCR workflow had excellent performance. In this particular experiment, MSRE+HC workflow provides better performance than MSRE+mPCR workflow (AUC of 0.999 vs 0.94). Higher sensitivity and specificity were observed using HC (Sens: 95%; Spec: 100%) compared to mPCR (Sens: 94%; Spec: 86%), as shown in Table 2 and FIG. 25A and FIG. 25B. In other experiments, MSRE+mPCR workflow may provide better performance than MSRE+HC workflow.

TABLE 2
Comparison of MSRE + HC and MSRE + PCR workflows

| | MSRE + HC | MSRE + mPCR |
|---|---|---|
| | (FIG. 25A) | (FIG. 25B) |
| Sensitivity | 95% | 94% |
| Specificity | 100% | 86% |
| AUC | 0.99 | 0.94 |

### MSRE Target Assay Investigation

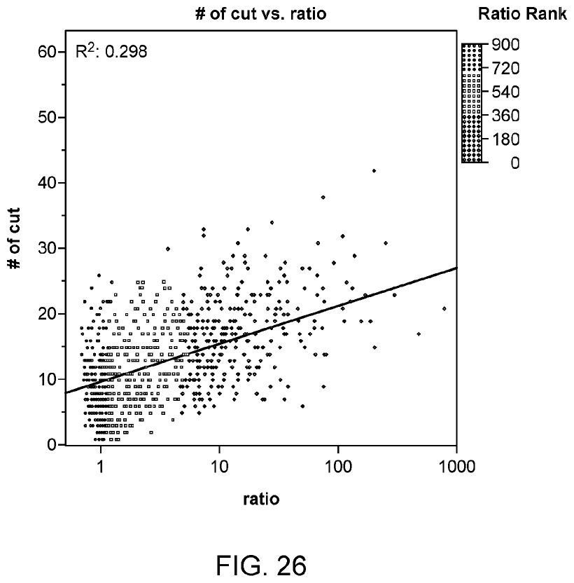

To understand the features of target assays that can provide a better differentiation of CRC vs healthy, the correlation of numbers of cut sites with the ratio of CRC/healthy mean DOR were examined. Consistent with findings in Example 2, assays with more MSRE cut sites provide larger differentiation (higher CRC/healthy DOR ratio, FIG. 26). It was observed that not all assays with multiple MSRE cut sites provide high CRC/healthy DOR ratio for differentiation. It is likely that not all CpG sites within these target regions are all hypermethylated in CRC.

### Comparison of Top 40 Targets Between MSRE+HC and MSRE+mPCR Workflows

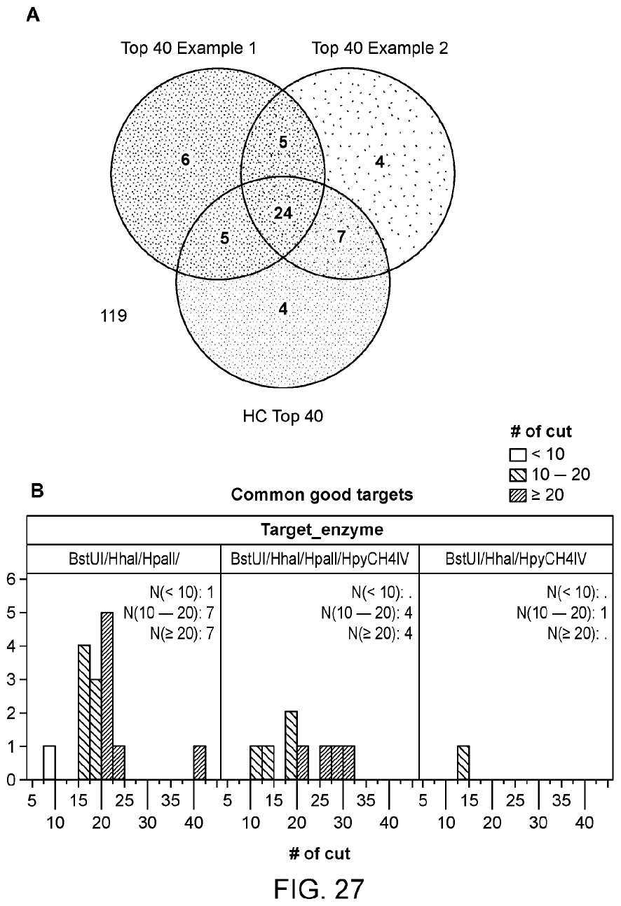

Cross-checking top 40 targets using HC with top 40 targets from previous mPCR studies using the same set of targets (Examples 1 and 2), 24/40 HC targets are found in both mPCR studies and 36/40 are found in at least one mPCR studies (FIG. 27A), suggesting that assays with higher CRC/healthy mDOR ratio are consistent among HC and mPCR workflows. Among top 24 common good target assays, 23/24 targets contain more than 10 cut sites. One of the good assays contains 8 cuts (FIG. 27B). The majority of good assays utilize BstUI/HhaI/HpaII, consistent with the prevalence of MSRE assays.

In conclusion, the MSRE+HC approach can be used to effectively differentiate CRC positive samples vs CRC negative or healthy samples. Methylation target selection can be further improved by using information of the MSRE cut sites and/or the methylation load.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by illustrative aspects, exemplary aspects and optional features, modification and variation of the concepts herein disclosed may be resorted to by those skilled in the art, and that such modifications and variations are considered to be within the scope of this invention as defined by the appended claims. The specific aspects provided herein are examples of useful aspects of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific aspects that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by illustrative aspects and optional features, modification and variation of the concepts herein disclosed may be resorted to by those skilled in the art, and that such modifications and variations are considered to be within the scope of this invention as defined by the appended claims.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A method for preparing deoxyribonucleic acid (DNA) molecules useful for determining a methylation status of a genomic region of interest, said method comprising:
ligating adapters to sample DNA molecules obtained or derived from a first sample from a first subject, thereby forming a plurality of adapted DNA molecules comprising adapted DNA molecules having methylation sensitive restriction enzyme (MSRE) recognition sites;
contacting the plurality of adapted DNA molecules with one or more MSREs, thereby generating MSRE-exposed, adapted DNA molecules, wherein at least a first plurality of the MSRE-exposed, adapted DNA molecules having one or more unmethylated MSRE recognition sites are cleaved by at least one of the MSREs, and wherein a second plurality of MSRE-exposed adapted DNA molecules are uncleaved by any of the MSREs, thus forming MSRE-exposed, uncleaved adapted DNA molecules; and
performing one or more amplification reactions to amplify one or more target regions from the MSRE-exposed, uncleaved adapted DNA molecules or copies thereof, wherein target region amplicons are formed if the one or more target regions are present in the MSRE-exposed, uncleaved adapted DNA molecules.

2. The method of claim 1, wherein each target region has one or more MSRE recognition sites.

3. A method according to claim 2, wherein the sample DNA molecules are circulating free DNA (cfDNA).

4. A method according to claim 3, wherein one or more target region amplicons are generated by one or more polymerase chain reactions (PCRs) using one or more primer pairs, and wherein at least one primer of each of the one or more primer pairs is a target-specific primer.

5. A method according to claim 4, wherein the method further comprises detecting the one or more target region amplicons.

6. A method according to claim 5, wherein the one or more target regions are a set of target regions, and wherein the one or more primer pairs are a set of primer pairs, each being configured for amplifying one target region of the set.

7. A method according to claim 6, wherein the method further comprises quantifying an amount for at least one of the one or more target region amplicons.

8. A method according to claim 7, wherein the one or more target regions are a set of target regions, and wherein the one or more primer pairs are a set of primer pairs, each being configured for amplifying one target region of the set.

9. A method according to claim 3, wherein one or more target region amplicons are generated by one or more linked target capture (LTC) reactions using one or more probe-dependent primer pairs.

10. A method according to claim 9, wherein the method further comprises detecting the one or more target region amplicons.

11. A method according to claim 10, wherein the one or more target regions are a set of target regions, and wherein the one or more probe-dependent primer pairs are a set of probe-dependent primer pairs, each being configured for amplifying one target region of the set.

12. A method according to claim 10, wherein the method further comprises quantifying an amount for at least one of the one or more target region amplicons.

13. A method according to claim 12, wherein the one or more target regions are a set of target regions, and wherein the one or more primer pairs are a set of primer pairs, each being configured for amplifying one target region of the set.

14. A method according to claim 3, wherein the performing one or more amplifications comprises performing a universal amplification, wherein at least a plurality of the MSRE-exposed, uncleaved, adapted cfDNA are amplified to generate amplicons, and wherein the method further comprises selectively enriching one or more target regions from the amplicons using one or more hybrid-capture probes to generate enriched target regions.

15. A method according to claim 14, wherein the method further comprises detecting the one or more enriched target regions.

16. A method according to claim 15, wherein the selectively enriching one or more target regions comprises selectively enriching a set of target regions, and wherein the one or more hybrid-capture probes are a set of hybrid-capture probes, each being configured for selectively enriching one target region of the set.

17. A method according to claim 14, wherein the method further comprises quantifying an amount for at least one of the one or more enriched target regions.

18. A method according to claim 17, wherein the one or more target regions are a set of target regions, and wherein the one or more hybrid capture probes are a set of hybrid capture probes, each being configured for selectively enriching one target region of the set.

19. A method for preparing DNA molecules useful for determining a methylation status of selected regions of the DNA molecules, comprising:
ligating adapters to circulating free DNA (cfDNA) obtained or derived from a first liquid sample, thereby forming a plurality of adapted cfDNA comprising adapted cfDNA having MSRE recognition sites;
contacting the plurality of adapted cfDNA with one or more MSREs, thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA;
performing a first amplification step wherein at least a plurality of the MSRE-exposed, uncleaved, adapted cfDNA are amplified;
performing a second amplification step to amplify a set of target regions from the amplified MSRE-exposed, uncleaved, adapted cfDNA, each target region having one or more MSRE recognition sites, wherein a target region of the set is amplified and thereby generating target region amplicons if the target region is present in one or more of the amplified MSRE-exposed, uncleaved, adapted cfDNA; and
quantifying an amount for at least some of the target region amplicons of the set by performing a next-generation sequencing reaction on clonally amplified target region amplicons, or amplicons derived therefrom.

20. A method according to 19, wherein the first amplification step comprises one or more PCRs using universal primers.

21. A method according to 20, wherein the second amplification step comprises one or more PCRs using a set of primer pairs, wherein each pair of the set is designed to amplify one target region having one or more MSRE recognition sites, and wherein at least one primer of each primer pair is a target-specific primer.

22. A method according to 20, wherein the second amplification step comprises one or more LTC reactions using a set of probe-dependent primer pairs, wherein each pair of the set is designed to amplify one target region having one or more MSRE recognition sites.

23. A method for preparing DNA molecules useful for determining a methylation status of selected regions of the DNA molecules, comprising:
ligating adapters to circulating free DNA (cfDNA) obtained or derived from a first liquid sample, thereby forming a plurality of adapted cfDNA comprising adapted cfDNA having MSRE recognition sites;
contacting the plurality of adapted cfDNA with one or more MSREs, thereby generating MSRE-exposed, adapted cfDNA, wherein a plurality of the MSRE-exposed, adapted cfDNA are not cleaved by the one or more MSREs, thereby forming MSRE-exposed, uncleaved, adapted cfDNA;
amplifying at least a plurality of the MSRE-exposed, uncleaved, adapted cDNA;
selectively enriching subsets of amplified MSRE-exposed, uncleaved, adapted cfDNA having one or more target regions using a set of hybrid capture probes, wherein each target region comprises one or more MSRE recognition sites, and wherein each probe of the set is designed to hybridize to one target region; and
quantifying an amount for at least some of the enriched subsets by performing a next-generation sequencing reaction on clonally amplified enriched subsets, or amplicons derived therefrom.

24. A method according to 23, wherein the amplifying comprises one or more PCRs using universal primers.

25. A method according to claim 24, wherein the one or more target regions are a set of target regions.

26. A method according to any one of claims 1 to 25, wherein each target region comprises two or more recognition sites recognized by the one or more MSREs.

27. A method according to any one of claims 1 to 24, wherein each target region comprises three or more recognition sites recognized by the one or more MSREs.

28. A method according to any one of claims 1 to 25, wherein each target region comprises between 1 and 10 recognition sites recognized by the one or more MSREs.

29. A method according to any one of claims 1 to 25, wherein each target region comprises between 2 and 8 recognition sites recognized by the one or more MSREs.

30. A method according to any one of claims 1 to 25, wherein the one or more MSREs is between 2 and 10 MSREs, and each target region comprises between 2 and 10 recognition sites recognized by at least 1 of the MSREs.

31. A method according to any one of claim 1-2, wherein the sample is a liquid sample.

32. The method of claim 31, wherein the liquid sample is a blood, plasma, serum, or urine sample.

33. The method of claim 31, wherein the liquid sample is a plasma sample.

34. The method of claim 19 or claim 23, wherein the quantifying the amount of at least some of the target region amplicons or of at least some of the enriched subsets, provides a quantity of an amount of circulating tumor DNA (ctDNA) in the first liquid sample.

35. The method of claim 19 or claim 23, wherein the method further comprises performing an additional amplification reaction that amplifies at least some of the target regions from the target region amplicons or from the enriched subsets of amplified MSRE-exposed, uncleaved, adapted cfDNA having one or more target regions, wherein the additional amplification reaction is a clonal amplification reaction to form clonally-amplified target region amplicons, and wherein the NGS is performed on the clonally-amplified target region amplicons.

36. The method of any one of claims 1 to 25, wherein the adapted DNA molecules having one or more MSRE recognition sites, or the adapted cfDNA having one or more MSRE recognition sites, each comprise two or more MSRE recognition sites.

37. The method of any one of claims 1 to 13, further comprising performing a universal PCR of the plurality of MSRE-exposed, adapted DNA molecules or the plurality of MSRE-exposed, adapted cfDNA, using a universal PCR primer pair comprising primers designed to bind universal primer binding sequences on the adapters, to generate amplified MSRE-exposed, adapted DNA molecules or MSRE-exposed, adapted cfDNA, before performing the one or more amplifications.

38. The method of claim 37, wherein at least one MSRE recognition site of the adapted DNA molecules or the adapted cfDNA having MSRE recognition sites is located outside a target region thereon.

39. The method of any one of claims 14-18, wherein at least one of the MSRE-exposed, adapted DNA molecules or the MSRE-exposed, adapted cfDNA, comprise one or more MSRE recognition sites outside a target region of the MSRE-exposed, adapted DNA molecules or the MSRE-exposed, adapted cfDNA.

40. The method of any one of claims 4 to 13, or 21 to 22, wherein both primers of at least one primer pair of the one or more primer pairs, of the set of primer pairs, or of the set of probe-dependent primer pairs, are target-specific primers.

41. The method of any one of claims 4 to 13, or 21 to 22, wherein both primers of a plurality of the one or more primer pairs, of the set of primer pairs, or of the set of probe-dependent primer pairs, are target-specific primers.

42. The method of any one of claims 4 to 13, or 21 to 22, wherein each primer of the one or more primer pairs, of the set of primer pairs, or of the set of probe-dependent primer pairs, is a target-specific primer.

43. The method of any one of claims 4 to 13, or 21 to 22, wherein at least one primer of at least one primer pair of the one or more primer pairs, or of the set of primer pairs, or of the set of probe-dependent primer pairs is designed to bind to a primer binding site on one of the adapters.

44. The method of any one of claims 4 to 13, or 21 to 22, wherein at least one primer of a plurality of primer pairs of the one or more primer pairs, or of the set of primer pairs, or of the set of probe-dependent primer pairs is designed to bind to a primer binding site on one of the adapters.

45. The method of any one of claims 4 to 13, or 21 to 22, wherein at least one primer of each primer pair of the one or more primer pairs, or of the set of primer pairs, or of the set of probe-dependent primer pairs is designed to bind to a primer binding site on one of the adapters.

46. The method of any one of claims 1 to 25, wherein one or more of the target regions, or set of the target regions, comprise one or more MSRE recognition sites overlapping a primer binding site of the one or more, or set of, primer binding sites.

47. The method of any one of claims 1 to 25, wherein the primer pairs are designed to bind to uncleaved versions of the adapted DNA molecules or the adapted cfDNA, but not cleaved adapted DNA molecules.

48. A method according to any one of claims 1 to 25, wherein the one or more MSREs is between 2 and 5 MSREs, and each target region comprises between 2 and 10 recognition sites recognized by at least 1 of the MSREs.

49. A method according to any one of claims 1 to 25, wherein the one or more MSREs is between 2 and 5 MSREs, and each target region comprises between 2 and 5 recognition sites recognized by at least 1 of the MSREs.

50. The method of any one of claims 1 to 2, wherein the first sample is a liquid sample.

51. The method of claim 50, wherein the liquid sample is a blood, serum, plasma, urine, vitreous, sputum, saliva, tears, perspiration, feces, bile, lymph, cervical mucus, or semen sample.

52. The method of claim 50, wherein the liquid sample is a blood, plasma, serum, or urine sample.

53. The method of 1 or 2, wherein the first sample is a blood sample or a derivative sample thereof.

54. The method of claim 50, wherein the first sample is a plasma sample.

55. The method of claim 50, wherein the first sample comprises DNA from a tumor.

56. The method of any one of claims 1 to 2, wherein the first sample is a sample from a cancer tissue.

57. The method of any one of claims 1 to 52, wherein the first sample comprises DNA from a tumor.

58. The method of any one of claims 1 to 52, wherein the first sample comprises DNA from a transplanted organ.

59. The method of any one of claims 1 to 52, wherein the first sample comprises DNA from a fetus.

60. The method of any one of claims 1 to 54, wherein the first subject is suspected or at risk of having a disease.

61. The method of claim 60, wherein the disease is a cancer.

62. The method of claim 61, wherein the cancer is selected from ovarian cancer, soft tissue sarcoma, peripheral T cell cancer, colorectal cancer, intrahepatic cholangiocarcinoma, glioblastoma, esophageal cancer, cutaneous T cell lymphoma, non-Hodgkin lymphoma, urothelial cancer, basal cell carcinoma, epithelioid sarcoma, pancreatic cancer, non-small cell lung carcinoma, Hodgkin lymphoma, renal cell carcinoma, mesothelioma, metastatic uveal melanoma, kidney cancer, blood cancer, HER2-expressing cancers, non-melanoma skin cancer, liposarcoma, hepatocellular carcinoma, small lymphocytic lymphoma, prostate cancer, breast cancer, anal cancer, marginal zone lymphoma, cutaneous squamous cell carcinoma, thyroid cancer, medullary thyroid cancer, triple-negative breast cancer, neuroendocrine prostate cancer, bladder cancer, paraganglioma, medulloblastoma, superficial basal cell carcinoma, head and neck squamous cell carcinoma, hematologic malignancies, melanoma, B-cell lymphoma, relapsed/refractory acute myeloid leukemia, angiosarcoma, bone sarcoma, refractory cervical cancer, cholangiocarcinoma, osteosarcoma, biliary tract cancer, castration-resistant prostate cancer, gastroesophageal adenocarcinomas, rhabdomyosarcoma, carcinoma, non-muscle invasive bladder cancer, uveal melanoma, small cell lung cancer, cervical cancer, primary open angle glaucoma, follicular lymphoma, synovial sarcoma, liver cancer, carcinosarcoma, leptomeningeal brain tumors, T-cell lymphoma, lymphoma, small cell lung cancer, mantle cell lymphoma, B-cell malignancies, endometrial cancer, myxoid/round cell liposarcoma, metastatic Merkel cell carcinoma, neuroblastoma, chronic lymphocytic leukemia, tenosynovial giant cell tumors, sarcoma, acute myeloid leukemia, skin cancer, nasopharyngeal carcinoma, relapsed/refractory Ewing sarcoma, bone cancer, glioma, salivary gland carcinoma, gastric cancer, benign tumor, low-grade serous ovarian cancer, metastatic breast cancer, multiple myeloma, diffuse large B cell lymphoma, relapsed/refractory lymphoma, metastatic colorectal cancer, advanced malignancies, and acute lymphoblastic leukemia.

63. The method of claim 61, wherein the cancer is selected from a cancer of the abdomen or abdominal wall, adrenal gland, anus, appendix, bladder, bone, brain, breast, cervix, chest wall, colon, diaphragm, duodenum, ear, endometrium, esophagus, fallopian tube, gallbladder, gastro-esophageal junction, head and neck, kidney, larynx, liver, lung, lymph node, malignant effusions, mediastinum, nasal cavity, omentum, ovarian, pancreas, pancreatobiliary, parotid gland, pelvis, penis, pericardium, peritoneum, pleura, prostate, rectum, salivary gland, skin, small intestine, soft tissue, spleen, stomach, thyroid, tongue, trachea, ureter, uterus, vagina, vulva, and whipple resection.

64. The method of claim 61, wherein the cancer is selected from lung cancer, breast cancer, bladder cancer, and colorectal cancer.

65. The method of claim 61, wherein the methylation status of the set of target regions is indicative of the presence or absence of the cancer.

66. The method of any one of claims 1 to 25 or 59, wherein the subject is a pregnant female.

67. The method of any one of claims 1 to 25 or 58, wherein the subject is a subject comprising an organ from another individual.

68. The method of any one of the preceding claims, wherein the method further comprises enriching the sample DNA molecules for DNA molecules that are between 70 and 500 base pairs in length.

69. The method of any one of the preceding claims, wherein the method further comprises enriching the sample DNA molecules for DNA molecules that are between 100 and 200 base pairs in length.

70. The method of any one of the preceding claims, wherein the method further comprises enriching the sample DNA molecules for DNA molecules that are between 130 and 170 base pairs in length.

71. The method of any one of claims 1 to 2, wherein before the ligating, the sample DNA molecules are fragmented to form fragmented DNA molecules.

72. The method of any one of the preceding claims, wherein the sample DNA molecules or the fragmented DNA molecules, are exposed to one or more polymerases and/or kinases to generate the nucleic acid derivatives generated therefrom.

73. The method of any one of preceding claims, wherein the ligating comprises ligating adapters to the nucleic acid derivatives generated therefrom.

74. The method of any one of the preceding claims, wherein the adapters are Y adapters.

75. The method of any one of the preceding claims, wherein the adapters each comprise a universal priming site.

76. The method of any one of the preceding claims, wherein the adapters do not include any MSRE recognition site.

77. The method of any one of the preceding claims, wherein the one or more MSREs are selected from one or more of HpaII, HhaI, HpyCH41V, or BstU1.

78. The method of any one of the preceding claims, wherein the contacting comprises contacting with two or more MSREs.

79. The method of claim 78, wherein the contacting comprises contacting with two or more MSREs in a single reaction.

80. The method of any one of the preceding claims, wherein the contacting comprises contacting with three or more MSREs.

81. The method of any one of the preceding claims, wherein the contacting comprises contacting with four or more MSREs.

82. The method of any one of claims 4 to 13, or 21 to 22, wherein the set of primer pairs is a set of between 2 and 1,000 primer pairs.

83. The method of any one of claims 4 to 13, or 21 to 22, wherein at least one of the primer pairs comprises a universal primer and a target-specific primer.

84. The method of any one of claims 4 to 13, or 21 to 22 wherein at least one of the primer pairs comprises two target-specific primers.

85. The method of any one of claims 4 to 13, or 21 to 22, wherein at least one of the primers comprises a sample index.

86. The method of any one of claims 1 to 25, wherein performing a PCR further comprises using primers comprising sequences that can be used for downstream sequencing reactions.

87. The method of any one of claim 4 or 21, wherein performing a PCR further comprises using primers comprising a sample index.

88. The method of claim 61, wherein the target regions each comprises a set of two or more MSRE recognition sites that are methylated in one or more cancers.

89. The method of claim 61, wherein the target regions each comprises three or more MSRE recognition sites that are predominantly methylated in a cancer.

90. The method of any one of claims 5 to 6, 10 to 11, or 15 to 16, wherein the method further comprises an additional amplification reaction that amplifies at least some of the amplified target region amplicons.

91. The method of claim 90, wherein the additional amplification reaction is a clonal amplification reaction to form clonally amplified target region amplicons.

92. The method of claim 91, wherein the detecting or the quantifying comprise performing a sequencing reaction on the clonally amplified target region amplicons.

93. The method of any one of claims 5 to 6, 10 to 11, or 15 to 16, wherein the detecting or the quantifying comprises performing a sequencing reaction.

94. The method of any one of claim 92 or 93, wherein the sequencing reaction is a next-generation sequencing (NGS) reaction.

95. The method of any one of claims 19 to 23 or 94, wherein the quantifying comprises counting sequence reads generated from the NGS reaction.

96. The method of claim 95, wherein the quantifying comprises determining a depth of read per target region for at least some of the target regions.

97. The method of claim 96, wherein the depth of read for each of the target region is normalized relative to a depth of read for a normalization sequence.

98. The method of claim 97, wherein the normalization sequence is a fully methylated DNA sequence.

99. The method of claim 98, wherein the fully methylated DNA sequence is derived from pUC19.

100. The method of claim 98, wherein the fully methylated DNA sequence is a synthetic sequence.

101. The method of claim 97, wherein the normalization sequence is a non-MSRE DNA sequence having no MSRE recognition site for any of the one or more MSREs.

102. The method of claim 97, wherein the non-MSRE DNA sequence is a human genomic DNA sequence.

103. The method of claim 97, wherein the non-MSRE DNA sequence is a synthetic sequence.

104. The method of claim 90, wherein the additional amplification reaction is a real-time PCR reaction.

105. The method of claim 90, wherein the additional amplification reaction is a digital PCR reaction.

106. The method of any one of the preceding claims, wherein:
the method further comprises performing the method on a second sample or a second liquid sample, and
determining the methylation status of the set of target regions in the first sample further comprises comparing the amount of target region amplicons derived from the first sample to the amount of target region amplicons derived from the second sample.

107. The method of claim 106, wherein the second sample is from a second subject.

108. The method of claim 107, wherein the first subject is suspected or at risk of having a disease, and wherein the second subject is not suspected or at risk of having the disease.

109. The method of claim 106, wherein the second sample is derived from a cell line.

110. The method of claim 106, wherein the method is performed on the first and second samples simultaneously.

111. The method of claim 106, wherein the second sample is from the first subject.

112. The method of claim 111, wherein the second sample is collected at a first timepoint and the first sample is collected at a second timepoint.

113. The method of claim 112, wherein the first timepoint precedes the second timepoint.

114. The method of any one of claims 5 to 25, wherein the determining comprises comparing the amount quantified for the target region amplicons derived from the first sample to a preset value or an amount quantified for target region amplicons derived from a second sample using the same method.

115. The method of any one of claims 7 to 8, 12 to 13, 17 to 18, 19 to 22 and 23 to 25, wherein the method further comprises in addition to a first performance of the method, a second performance of the method performed at the same time as, or a different time than the first performance, wherein the second performance does not comprise the contacting step, and wherein the method further comprises comparing the amount quantified in the first performance to the amount quantified in the second performance to determine the methylation status of the target nucleic acid.

116. The method of claim 115, wherein the amount is not greater than an amount of amplicons generated using a negative control nucleic acid sample having no methylated MSRE recognition sites.

117. The method of claim 115, wherein the amount is greater than an amount of amplicons generated using a negative control nucleic acid sample having no methylated MSRE recognition sites.

118. The method of any one of the preceding claims, wherein the method is capable of detecting fully methylated DNA molecules present at 1.0% (by mass) or less in a mixture of DNA molecules.

119. The method of any one of the preceding claims, wherein the method is capable of differentiating samples having 1.0% (by mass) or less fully methylated DNA molecules from samples having 0% of the fully methylated DNA molecules.

120. The method of any one of the preceding claims, wherein the method is capable of differentiating samples having 0.1% (by mass) or less fully methylated DNA molecules from samples having 0% of the fully methylated DNA molecules.

121. The method of any one of the preceding claims, wherein the method is capable of differentiating samples having 0.05% (by mass) or less fully methylated DNA molecules from samples having 0% of the fully methylated DNA molecules.

122. The method of any one of the preceding claims, wherein circulating tumor DNA (ctDNA) is present in 1% or less of the total circulating free DNA (cfDNA) in the sample.

123. The method of any one of the preceding claims, wherein circulating tumor DNA (ctDNA) is present in 0.1% or less of the total circulating free DNA (cfDNA) in the sample.

124. The method of any one of the preceding claims, wherein circulating tumor DNA (ctDNA) is present in 0.01% or less of the total circulating free DNA (cfDNA) in the sample.

125. A method according to any one of the preceding claims, wherein the adapters each further comprise a molecular barcode.

126. The method of claim 125, wherein the number of adapters having different molecular barcodes is between 10 to 1,000, and wherein the ratio of the total number of sample DNA or cfDNA molecules to the number of different molecular barcodes in the ligation reaction is at least 1,000:1.

127. The method of claim 125, wherein the ratio of the total number of sample DNA or cfDNA molecules to the number of different molecular barcodes in the ligation reaction is at least 10,000:1.

128. The method of any one of the preceding claims, wherein the sample DNA obtained or derived from the first sample comprises a mixture of hypermethylated DNA and hypomethylated DNA.

129. The method of claim 128, wherein the adapters are ligated to the mixture of hypermethylated DNA and hypomethylated DNA.
